SEQUENCE LISTING (19 sequences)

SEQ ID NO: 1 - protein, 8 aa, unidentified
GYTFTSYY

SEQ ID NO: 2 - protein, 8 aa, unidentified
INPSNAGT

SEQ ID NO: 3 - protein, 12 aa, unidentified
TREGNPYYTMNY

SEQ ID NO: 4 - protein, 12 aa, unidentified
QSLLNSGNQKTY

SEQ ID NO: 5 - skipped

SEQ ID NO: 6 - protein, 9 aa, unidentified
QNDHSYPFT

SEQ ID NO: 7 - protein, 119 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 8 - protein, 113 aa, unidentified
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIK

SEQ ID NO: 9 - protein, 449 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 440 aa, unidentified
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 449 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 449 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 446 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 446 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 10 aa, unidentified
FLSRPTEKTI

(19) United States
(12) Patent Application Publication
(10) Pub. No.: US 2026/0256918 A1
(43) Pub. Date: Sep. 3, 2026

(54) METHOD OF TREATING NEUROLOGICAL DISORDER

(71) Applicant: AlaMab Therapeutics, Inc., Princeton, NJ (US)

(72) Inventor: Yanfeng Zhang, Phoenixville, PA (US)

(21) Appl. No.: 19/549,989

(22) Filed: Feb. 25, 2026

Related U.S. Application Data
(60) Provisional application No. 63/765,394, filed on Feb. 28, 2025.

(51) Int. Cl. A61K 45/06; A61K 31/513; A61K 38/00

(52) U.S. Cl. CPC A61K 45/06; A61K 31/513; A61K 38/00; C07K 2317/565

(57) ABSTRACT

The present disclosure provides compositions and methods of treating spinal cord injury in a subject (e.g., a human subject). The method comprises blocking the opening of Cx43 hemichannels in astrocytes by, e.g., using a composition comprising an anti-Cx43 antibody.

### CROSS-REFERENCE TO RELATED APPLICATIONS

This application claims the benefit of priority of U.S. Provisional Application No. 63/765,394, filed Feb. 28, 2025, the entire contents of which are incorporated herein by reference.

### INCORPORATION BY REFERENCE OF SEQUENCE LISTING

The instant application contains a Sequence Listing, which has been submitted via Patent Center. The Sequence Listing titled 172628-205002_US_SL.xml, which was created on Feb. 24, 2026 and is 21,271 bytes in size, is hereby incorporated by reference in its entirety.

### BACKGROUND

Acute spinal cord injury is a complex disease that severely affects quality of life, usually starting from a sudden traumatic impact to the spine, leading to fracture vertebral or dislocation. These injuries have serious negative impacts on patients' physical, psychological, and psychosocial well-being. Meanwhile, the harmful neurological effects induced by SCI include ammonia poisoning, oxidative stress, ion and metabolic loss of balance, and primary and secondary injuries caused by inflammation and ischaemia. Conversely, this injury leads to necrosis and cell death. Secondary injury mechanisms play a significant role in the loss of neurological function after trauma. The neuroinflammatory process after injury leads to the activation of astrocytes and the formation of glial scars, creating an environment that is not easily permeable, which prevents axonal regeneration and thereby affects the recovery of functions of the entire peripheral nervous system.

From Jan. 1, 1978 to Aug. 30, 2017, an incomplete statistical analysis of the epidemiology of acute spinal cord injury in China was conducted, and the annual illness rate of acute spinal cord injury in China was 13-60 subjects per million. Illness rates vary across different regions, with the highest annual incidence rate in Beijing being 60.2 per million, 4.4 times that of Shanghai. The male-to-female ratio of patients with spinal cord injury in China ranges from 0.99 to 15.3:1. The current prevalence is higher in males than in females, but the incidence rate in females has been slightly increasing year by year. The mean age of patients with acute spinal cord injury ranged from 34.7 to 54.4 years. A total of 32 studies were included, with 28 studies reporting the injury rates of the cervical and thoracic vertebrae. Among them, the proportion of cervical vertebra injury was 4.9%-83.6%, and the proportion of thoracic vertebra injury was 3.8%-82.0%. Twenty studies conducted a pooled analysis of the proportions of ASIA injury grades, among which Grade A accounted for 9.4%-52.5%, Grade B accounted for 1.7%-2.0%, Grade C accounted for 4.5%-38.5%, and Grade D accounted for 15.0%-68.4%. The injury proportion of Grade A was highest, and the injury proportion of Grade E was lowest (Witiw et al., J Spinal Disord Tech, 28(6): 202-210 (2015)).

According to the 2018 Evidence-Based Clinical Diagnostic and Treatment Guidelines for spinal cord injury by the Chinese Medical Doctor Association, high-dose steroids and ganglioside therapy for spinal cord injury are not considered as routine treatment protocols. It is recommended to use neuroprotective drugs and neurotrophic drugs (nerve growth factor, adenosylcobalamin); low-dose hormone use is allowed (methylprednisolone 80 mg ivgtt qd d1-3). Preferably, no hormones should be used. There are currently no approved treatments for SCI.

In recent years, the medical management of SCI has made great progress, significantly improving patients' diagnostic rates, disease stabilization rates, survival rates, and quality of life. However, little progress has been made in developing treatment protocols that improve neurological outcomes for patients with acute spinal cord injury. This also further reflects the complexity of the pathophysiology of SCI and the various biochemical and physiological changes that occur in SCI. Treatments that reduce or prevent secondary injury can significantly improve functional recovery and quality of life in patients with SCI.

Despite the development of medical management for SCI, there remains an unmet need for therapies for effective treatment of SCI, acute SCI and inhibiting secondary SCI.

### SUMMARY

One aspect of the disclosure provides a method for treating spinal cord injury in a subject in need thereof, comprising administering to the subject an anti-connexin 43 (Cx43) antibody. In some embodiments, the anti-Cx43 antibody comprises the following heavy chain CDR sequences and light chain CDR sequences:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5; and
LCDR3: SEQ ID NO: 6.

In some embodiments, the anti-Cx43 antibody is administered at a dosage from 100 mg to 5000 mg. In some embodiments, the dosage is between 200 mg to 4800 mg. In some embodiments, the dosage is about 200 mg, about 600 mg, about 1200 mg, about 2400 mg, or about 4800 mg.

In one embodiment, the dosage is about 200 mg. In another embodiment, the dosage is about 600 mg. In another embodiment, the dosage is about 1200 mg. In another embodiment, the dosage is about 2400 mg. In yet another embodiment, the dosage is about 4800 mg.

In some embodiments, the subject is a human. In some embodiments, the SCI is C3-C7 SCI. In some embodiments, the SCI is acute SCI. In some embodiments, the SCI is complete SCI. In other embodiments, the SCI is incomplete SCI.

In some embodiments, the anti-Cx43 antibody is administered intravenously or subcutaneously. In some embodiments, the anti-Cx43 antibody is administered intravenously. In some embodiments, the anti-Cx43 antibody is administered within 60 minutes.

In some embodiments, at least one evaluation indicator of spinal cord injury is improved after the anti-Cx43 antibody is administered. In some embodiments, the at least one indicator is selected from a group consisting of: ASIA motor score, total motor score (right), total motor score (left), upper limb motor score, upper limb motor score, ASIA sensatory score, injury grading, pinprick score, light touch score, and pain visual analogue scale.

In some embodiments, the subject does not experience severe adverse event after the anti-Cx43 antibody is administered.

In some embodiments, the subject is anti-drug antibody (ADA) negative after the anti-Cx43 antibody is administered.

In some embodiments, the anti-Cx43 antibody comprises heavy chain variable sequence of SEQ ID NO: 7, and/or light chain variable sequence of SEQ ID NO: 8. In some embodiments, the anti-Cx43 antibody comprises heavy chain sequence of any one of SEQ ID NOs: 9 and 11-18, and/or light chain variable sequence of SEQ ID NO: 10. In some embodiments, the anti-Cx43 antibody comprises a heavy chain sequence having the amino acid sequence of SEQ ID NO: 9, and/or a light chain sequence having the amino acid sequence of SEQ ID NO: 10. In some embodiments, the anti-Cx43 antibody blocks the opening of Cx43 hemichannel in the spinal cord astrocytes of the subject.

### BRIEF DESCRIPTION OF DRAWINGS

FIG. 1 depicts predicted human plasma concentration-time curves after multiple doses of ALMB-0166 administered once weekly at different dosing levels.

FIG. 2 is a flow chart depicting the different arms (subject distribution) of the trial.

FIG. 3 is mean±SE time curve showing changes from baseline in total sensory score for the full analysis set.

FIG. 4 is mean±SE time curve of change from baseline in total sensory score—left for the full analysis set.

FIG. 5 is mean±SE time curve of change from baseline in total sensory score—right for the full analysis set.

FIG. 6 is mean±SE time curve of changes from baseline in total pinprick scores for the full analysis set.

FIG. 7 is mean±SE time curve of changes from baseline in total light touch score for the full analysis set.

FIG. 8 is mean±SE time curve of changes from baseline in total motor score for the full analysis set.

FIG. 9 is mean±SE time curve of changes from baseline in total motor score—left for the full analysis set.

FIG. 10 is mean±SE time curve of changes from baseline in total motor score—right for the full analysis set.

FIG. 11 is mean±SE time curve of changes from baseline in upper limb motor score for the full analysis set.

FIG. 12 is mean±SE time curve of changes from baseline in lower limb motor score for the full analysis set.

FIGS. 13A-13B are mean ALMB-0166 time-concentration curves (PKCS) after administration of various doses. FIG. 13A shows the linear pharmacokinetic (PK) curve after administration of 3 mg/kg (or 200 mg), 600 mg, 1200 mg, 2400 mg, and 4800 mg. FIG. 13B shows the semi-log PK curve after administration of the same various doses.

FIGS. 14A-14C are plots of ALMB-0166 PK parameters vs. dose (PKPS). FIG. 14A is the Cₘₐₓ plot of dose groups including 3 mg/kg (or 200 mg), 600 mg, 1200 mg, 2400 mg, and 4800 mg. FIG. 14B and FIG. 14C are the AUC0-t and AUC0-∞ of the same dose groups, respectively.

FIG. 15 is mean titer-time curve of immunogenicity evaluation.

### DETAILED DESCRIPTION

The present disclosure provides methods and compositions for treating neurological disorder (e.g., spinal cord injury) in a subject in need thereof, comprising administering an anti-Cx43 antibody to the subject or patient. In some embodiments, the anti-Cx43 antibody blocks or inhibits the opening of Cx43 hemichannel in cells associated with the neurological disorder (e.g., astrocyte). In some embodiments, the anti-Cx43 antibody comprises specific CDR amino acid sequences. In some embodiments, the anti-Cx43 antibody is administered according to a dosing regimen.

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or +1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “providing” is used according to its ordinary meaning to supply or furnish for use. In some embodiments, the protein (e.g., an antibody) is provided directly by administering the protein, while in other embodiments, the protein (e.g., an antibody) is effectively provided by administering a nucleic acid that encodes the protein. In certain aspects the invention contemplates compositions comprising various combinations of nucleic acid, antigens, peptides, and/or epitopes.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “percent sequence identity” or “sequence identity” refers to the degree of identity between any given query sequence and a subject sequence. A percent identity for any query nucleic acid or amino acid sequence, relative to another subject nucleic acid or amino acid sequence can be determined using tools and technologies known in the art, for example, NCBI BLAST.

The term “pharmaceutical formulation” refers to a preparation that contains a therapeutic agent (e.g., an anti-Cx43 antibody). In such form as to permit the biological activity of the antibody to be effective, and which contains no additional components which are unacceptably toxic to a subject to which the formulation would be administered.

As used herein, the term “antigen” is a molecule capable of being bound by an antibody or T-cell receptor. In certain embodiments, binding moieties other than antibodies are engineered to specifically bind to an antigen, e.g., aptamers, avimers, and the like.

As used herein, the term “specifically binds” is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 1010 M⁻¹, or about 1010 M⁻¹ to about 1011 M⁻¹. Antibodies may, for example, bind with an EC so of 50 nM or less, 10 nM or less, 1 nM or less, 100 pM or less, or more preferably 10 pM or less. As kwon in the art, a variety of immunoassay formats may be used to select antibodies specifically immunoreactive with an antigen. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

An “affinity matured” antibody has one or more alterations in one or more hypervariable regions thereof which result an improvement in the affinity of the antibody for antigen, compared to a parent antibody which does not possess those alteration(s). In one aspect, affinity matured antibodies will have nanomolar or even picomolar affinities for the target antigen. Affinity matured antibodies are produced by procedures known in the art. Marks et al., Biol. Technology, 10:779-783 (1992) describes affinity maturation by VH and VL domain shuffling. Random mutagenesis of CDR and/or framework residues is described by: Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

The term “affinity” refers to the strength of a binding reaction between a binding domain of an antibody and an epitope. It is the sum of the attractive and repulsive forces operating between the binding domain and the epitope. The term affinity, as used herein, refers to the dissociation constant KD.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. In one embodiment, an epitope is a region of an antigen that is bound by an antibody. In certain embodiments, an antibody is said to specifically bind an antigen when it preferentially recognizes its target antigen in a complex mixture of proteins and/or macromolecules. Methods for epitope mapping are well known in the art, such as X-ray co-crystallography, array-based oligo-peptide scanning, site-directed mutagenesis, high throughput mutagenesis mapping and hydrogen-deuterium exchange. Epitopes can be formed both from contiguous amino acids, or noncontiguous amino acids juxtaposed by tertiary folding of a protein. Epitopes formed from contiguous ammo acid are typically retained on exposure to denaturing solvents, whereas epitopes formed by tertiary folding are typically lost on treatment with denaturing solvents. An epitope typically includes at least 3, and more usually, at least 5 or 8-10 amino acid in a unique spatial conformation.

The terms “reduce,” “inhibit” and “block” as used interchangeably herein, refer to any statistically significant decrease in biological activity (e.g., hemichannel opening). For example, “reduction” can refer to a decrease of about 10%, 20%, 30%, 40%, 50%, 60%, 70%, 80%, 90%, or 100% in biological activity.

### II. Antibodies

The term “antibody” or “antibodies” herein is used in the broadest sense and specifically covers full length antibody, antibody peptide(s) or immunoglobulin(s), monoclonal antibodies, chimeric antibodies, polyclonal antibodies, human antibodies, humanized antibodies and antibodies from non-human species, including human antibodies derived from a human germline immunoglobulin sequence transduced into the non-human species, e.g., mouse, sheep, chicken or goat, recombinant antigen binding forms such as monobodies and diabodies, multi-specific antibodies (e.g., bispecific antibodies), and individual antigen binding fragments of any of the foregoing, e.g., of an antibody or the antibody from which it is derived, including dAbs, Fv, scFv, Fab, F(ab)′2, Fab′.

Antibody-like binding peptidomimetics are also contemplated in the methods described herein. Liu et al. (2003) describe “antibody-like binding peptidomimetics” (ABiPs), which are peptides that act as pared-down antibodies and have certain advantages of longer serum half-life as well as less cumbersome synthesis methods.

The term “monoclonal antibody” as used herein refers to an antibody obtained from a population of substantially homogeneous antibodies, i.e., the individual antibodies comprising the population are identical and/or bind the same epitope. The modifier “monoclonal” indicates the character of the antibody as being obtained from a substantially homogeneous population of antibodies, and is not to be construed as requiring production of the antibody by any particular method.

“Antigen binding fragments” of an antibody preferably comprise at least the variable regions of the heavy and/or light chains of an anti-Cx43 antibody. For example, an antigen binding fragment of anti-Cx43 antibodies can comprise amino acid sequences of SEQ ID NOs: 7 and 8. Examples of such antigen binding fragments include Fab fragments, Fab′ fragments, Fv fragments, scFv and F(ab′)2 fragments. Antigen binding fragments of an antibody can be produced by enzymatic cleavage or by recombinant techniques. For instance, papain or pepsin cleavage can be used to generate Fab or F(ab′)2 fragments, respectively. Antibodies can also be produced in a variety of truncated forms using antibody genes in which one or more stop codons have been introduced upstream of the natural stop site. For example, a recombinant construct encoding the heavy chain of an F(ab′)2 fragment can be designed to include DNA sequences encoding the CH1 domain and hinge region of the heavy chain. In one aspect, antigen binding fragments blocks or inhibits the opening of Cx43 hemi-channel in a subject and the effects associated with opening of Cx43 hemi-channel.

A “multi-specific antibody” as used herein refers to an artificial antibody that has two or more different portions, with each portion including the antigen binding region of an antibody to a different antigen or epitope. The portions of the multi-specific antibody can be, for example, full-length antibodies or antibody binding fragments. Bispecific or bifunctional antibodies are multi-specific antibodies that have two different portions (e.g., two different heavy and light chain pairs, or two different antibody binding fragments) that bind two different antigens or epitopes. Multi-specific antibodies can be produced by a variety of methods, including fusion of hybridomas or ligation of antibody binding fragments. For example, Songsivilai and Lachmann, Clin Exp Immunol., 79: 315-21 (1990); Kostelny et al., J. Immunol., 148: 1547-53 (1992). In some cases, a multi-specific antibody (e.g., a bispecific antibody) includes at least one portion an anti-Cx43 antibody that blocks or inhibits the opening of Cx43 hemi-channel in a subject and the effects associated with opening of Cx43 hemi-channel.

A “therapeutic monoclonal antibody” is an antibody used for therapy of a human subject. Therapeutic monoclonal antibodies disclosed herein include anti-Cx43 antibodies. Antibody “effector functions” refer to those biological activities attributable to the Fc region (a native sequence Fc region or amino acid sequence variant Fc region) of an antibody. Examples of antibody effector functions include C1q binding, complement dependent cytotoxicity, Fc receptor binding, antibody-dependent cell-mediated cytotoxicity (ADCC), phagocytosis, down regulation of cell surface receptors (e.g., B cell receptor; BCR), and the like. To assess ADCC activity of a molecule of interest, an in vitro ADCC assay, such as those described in U.S. Pat. No. 5,500,362 or 5,821,337 may be performed.

An anti-Cx43 antibody of the disclosure can also be conjugated to at least one agent or moiety to form an antibody conjugate. The antibody may be linked to the agent covalently or non-covalently. The agent or moiety can increase the diagnostic or therapeutic potential of the antibody, and include, but are not limited to, effector or reporter molecules. Effector molecules include molecules with a desired activity (e.g., cytotoxic activity). Non-limiting examples of effector molecules that can be attached to an antibody include toxins, small molecule drugs, therapeutic enzymes, cytokines, antibiotics, radiolabeled nucleotides, and the like. Reporter molecules are molecules that can be detected by an assay, and include, without limitation, enzymes, radioactive labels, haptens, fluorescent labels, phosphorescent molecules, chemically luminescent molecules, chromophores, luminescent molecules, photoaffinity molecules, colored particles, or ligands (e.g., biotin).

Depending on the amino acid sequence of the constant domain of their heavy chains, full length antibodies can be assigned to different “classes.” There are five major classes of full-length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ, respectively. The subunit structures and three-dimensional configurations of different classes of antibodies are well known. The “light chains” of antibodies from any vertebrate species can be assigned to one of two clearly distinct types, called kappa (κ) and lambda (λ), based on the amino acid sequences of their constant domains.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “hypervariable region” when used herein refers to the amino acid residues of an antibody which are responsible for antigen binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mal. Biol. 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

As will be appreciated by those in the art, the CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

Amino acid substitutions are typically of single residues; insertions usually will be on the order of from about one to about twenty amino acid residues, although considerably larger insertions may be tolerated. Deletions range from about one to about twenty amino acid residues, although in some cases deletions may be much larger.

Substitutions, deletions, insertions or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances.

The term “Fab” or “Fab region,” as used herein, is meant the polypeptide that comprises the VH, CH1, VL, and CL immunoglobulin domains. Fab may refer to this region in isolation, or this region in the context of a full-length antibody, antibody fragment or Fab fusion protein, or any other antibody embodiments as outlined herein.

The term “Fv,” “Fv fragment” or “Fv region,” as used herein, refers to a polypeptide that comprises the VL and VH domains of a single antibody.

The term “framework,” as used herein, is refers to the region of an antibody variable domain exclusive of those regions defined as CDRs. Each antibody variable domain framework can be further subdivided into the contiguous regions separated by the CDRs (FR1, FR2, FR3 and FR4).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. For further details, see Jones et al., Nature 321:522-525 (1986); Riechmann et al., Nature, 332:323-329 (1988); and Presta, Curr. Op. Struct. Biol., 2:593-596 (1992).

An “isolated” antibody is one which has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

### Anti-Cx43 Antibodies

Certain aspects of the present disclosure provide compositions and methods of treating a neurological disorder (e.g., spinal cord injury) in a subject. The methods comprise administering an anti-Cx43 antibody that specifically binds Cx43 hemichannel and blocks or inhibits the opening of it in a cell associated with neurological disorder, e.g., astrocyte.

The anti-Cx43 antibody can be any antibody specifically binding Cx43 known in the art. In various embodiments, the anti-Cx43 antibody used in the methods comprises an HCDR1 amino acid sequence having at least 90%, at least 95%, or at least 99% sequence identity to SEQ ID NO: 1, an HCDR2 amino acid sequence having at least 90%, at least 95%, or at least 99% sequence identity to SEQ ID NO: 2, and an HCDR3 amino acid sequence having at least 90%, at least 95%, or at least 99% sequence identity to SEQ ID NO: 3; and/or an LCDR1 amino acid sequence having at least 90%, at least 95%, or at least 99% sequence identity to SEQ ID NO: 4, an LCDR2 amino acid sequence having at least 90%, at least 95%, or at least 99% sequence identity to SEQ ID NO: 5, an LCDR3 amino acid sequence having at least 90%, at least 95%, or at least 99% sequence identity to SEQ ID NO: 6. In some embodiments, the anti-Cx43 antibody comprises an HCDR1 amino acid sequence identical to SEQ ID NO: 1, an HCDR2 amino acid sequence identical to SEQ ID NO: 2, and an HCDR3 amino acid sequence identical to SEQ ID NO: 3; and/or an LCDR1 amino acid sequence identical to SEQ ID NO: 4, an LCDR1 amino acid sequence identical to SEQ ID NO: 5, and an LCDR3 amino acid sequence identical to SEQ ID NO: 6.

In various embodiments, the anti-Cx43 antibody used in the method comprises a heavy chain having an amino acid sequence that has at least 80%, at least 85%, at least 90%, at least 95%, or at least 99% sequence identity to any one of SEQ ID NOs: 9 and 11-18; and/or a light chain having an amino acid sequence that has at least 80%, at least 85%, at least 90%, at least 95%, or at least 99% sequence identity to any one of SEQ ID NO: 10. In some embodiments, the anti-Cx43 antibody comprises a heavy chain having an amino acid sequence that has at least 80%, at least 85%, at least 90%, at least 95%, or at least 99% sequence identity to SEQ ID NO: 9; and a light chain having an amino acid sequence having at least 80%, at least 85%, at least 90%, at least 95%, or at least 99% sequence identity to any one of SEQ ID NO: 10. In some other embodiments, the anti-Cx43 antibody comprises a heavy chain having an amino acid that has at least 80%, at least 85%, at least 90%, at least 95%, or at least 99% sequence identity to SEQ ID NO: 11; and a light chain having an amino acid that has at least 80%, at least 85%, at least 90%, at least 95%, or at least 99% sequence identity to any one of SEQ ID NO: 10. In some other embodiments, the anti-Cx43 antibody comprises a heavy chain having an amino acid that has at least 80%, at least 85%, at least 90%, at least 95%, or at least 99% sequence identity to SEQ ID NO: 12; and a light chain having an amino acid that has at least 80%, at least 85%, at least 90%, at least 95%, or at least 99% sequence identity to any one of SEQ ID NO: 10. In yet some other embodiments, the anti-Cx43 antibody comprises a heavy chain having an amino acid that has at least 80%, at least 85%, at least 90%, at least 95%, or at least 99% sequence identity to SEQ ID NO: 13; and a light chain having an amino acid that has at least 80%, at least 85%, at least 90%, at least 95%, or at least 99% sequence identity to any one of SEQ ID NO: 10. In yet some other embodiments, the anti-Cx43 antibody comprises a heavy chain having an amino acid that has at least 80%, at least 85%, at least 90%, at least 95%, or at least 99% sequence identity to SEQ ID NO: 14; and a light chain having an amino acid that has at least 80%, at least 85%, at least 90%, at least 95%, or at least 99% sequence identity to any one of SEQ ID NO: 10. In yet some other embodiments, the anti-Cx43 antibody comprises a heavy chain having an amino acid that has at least 80%, at least 85%, at least 90%, at least 95%, or at least 99% sequence identity to SEQ ID NO: 15; and a light chain having an amino acid that has at least 80%, at least 85%, at least 90%, at least 95%, or at least 99% sequence identity to any one of SEQ ID NO: 10. In some embodiments, the anti-Cx43 antibody comprises a heavy chain having an amino acid that has at least 80%, at least 85%, at least 90%, at least 95%, or at least 99% sequence identity to SEQ ID NO: 16; and a light chain having an amino acid that has at least 80%, at least 85%, at least 90%, at least 95%, or at least 99% sequence identity to any one of SEQ ID NO: 10. In some embodiments, the anti-Cx43 antibody comprises a heavy chain having an amino acid that has at least 80%, at least 85%, at least 90%, at least 95%, or at least 99% sequence identity to SEQ ID NO: 17; and a light chain having an amino acid that has at least 80%, at least 85%, at least 90%, at least 95%, or at least 99% sequence identity to any one of SEQ ID NO: 10. In some other embodiments, the anti-Cx43 antibody comprises a heavy chain having an amino acid that has at least 80%, at least 85%, at least 90%, at least 95%, or at least 99% sequence identity to SEQ ID NO: 18; and a light chain having an amino acid that has at least 80%, at least 85%, at least 90%, at least 95%, or at least 99% sequence identity to any one of SEQ ID NO: 10.

In some embodiments, the anti-Cx43 antibody comprises a heavy chain having an amino acid sequence identical to any one of SEQ ID NOs: 9 and 11-18, and/or a light chain having an amino acid sequence identical to any one of SEQ ID NO: 10. In one embodiment, the anti-Cx43 antibody comprises a heavy chain having an amino acid sequence identical to SEQ ID NO: 9, and a light chain having an amino acid sequence identical to any one of SEQ ID NO: 10. In one embodiment, the anti-Cx43 antibody comprises a heavy chain having an amino acid sequence identical to SEQ ID NO: 11, and a light chain having an amino acid sequence identical to any one of SEQ ID NO: 10. In another embodiment, the anti-Cx43 antibody comprises a heavy chain having an amino acid sequence identical to SEQ ID NO: 12, and a light chain having an amino acid sequence identical to any one of SEQ ID NO: 10. In yet another embodiment, the anti-Cx43 antibody comprises a heavy chain having an amino acid sequence identical to SEQ ID NO: 13, and a light chain having an amino acid sequence identical to any one of SEQ ID NO: 10. In yet another embodiment, the anti-Cx43 antibody comprises a heavy chain having an amino acid sequence identical to SEQ ID NO: 14, and a light chain identical to any one of SEQ ID NO: 10. In yet another embodiment, the anti-Cx43 antibody comprises a heavy chain having an amino acid sequence identical to SEQ ID NO: 15, and a light chain having an amino acid sequence identical to any one of SEQ ID NO: 10. In yet another embodiment, the anti-Cx43 antibody comprises a heavy chain identical to SEQ ID NO: 16, and a light chain having an amino acid sequence identical to any one of SEQ ID NO: 10. In yet another embodiment, the anti-Cx43 antibody comprises a heavy chain having an amino acid sequence identical to SEQ ID NO: 17, and a light chain having an amino acid sequence identical to any one of SEQ ID NO: 10. In yet another embodiment, the anti-Cx43 antibody comprises a heavy chain having an amino acid sequence identical to SEQ ID NO: 18, and a light chain having an amino acid sequence identical to any one of SEQ ID NO: 10.

In various embodiments, provided herein is an antibody that binds an epitope located within, partially or entirely, the amino acid sequence of FLSRPTEKTI (SEQ ID NO: 19). In some embodiments, the epitope can comprise one or more amino acids selected from the group consisting of F1, S3, R4, P5, T6, E7, K8, T9, or 110 of SEQ ID NO: 19. In one embodiment the epitope consists of F1, S3, R4, P5, T6, E7, K8, T9 and 110 of SEQ ID NO: 19. In some embodiments, the epitope can include all ten amino acids of SEQ ID NO: 19. In certain embodiments, the epitope consists of all ten amino acids of SEQ ID NO: 19.

The anti-Cx43 antibody is substantially pure and desirably substantially homogeneous (i.e., free from contaminating proteins, etc.). “Substantially pure” antibody means a composition comprising at least about 90% antibody by weight, based on total weight of the protein in the composition, at least about 95% or 97% by weight. “Substantially homogeneous” antibody means a composition comprising protein wherein at least about 99% by weight of protein is specific antibody, e.g., anti-Cx43 antibody, based on total weight of the protein.

In some embodiments, the anti-Cx43 antibody is a humanized antibody. In some embodiments, the anti-Cx43 antibody is a monoclonal antibody. In some embodiments, the anti-Cx43 antibody is a humanized monoclonal antibody.

### Pharmaceutical Formulation Comprising Anti-Cx43 Antibody

One aspect of the present disclosure provides an anti-Cx43 antibody pharmaceutical formulation for treating a neurological disorder (e.g., spinal cord injury) in a subject. The anti-Cx43 antibody can be a full antibody or an antigen-binding fragment thereof.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent may be coated in a material to protect the component from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, or by the use of surfactants, such as polysorbate, sodium dodecyl sulfate, and nonionic surfactant. In many cases, it may be useful to include isotonic agents, for example, sugars, polyalcohols such as mannitol, sorbitol, or sodium chloride in the composition. Prolonged absorption of the injectable compositions can be brought about by including in the composition, an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration. Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol phenol sorbic acid, and the like. it may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions described herein can also have various viscosities or osmolarities. Methods of measuring viscosity of antibody formulations are known to those in the art, and can include, e.g., a rheometer (e.g., Anton Paar MCR 301 Rheometer with either a 50 mm, 40 mm or 20 mm plate accessory). Methods of measuring osmolarity of antibody formulations are known to those in the art, and can include, e.g., an osmometer (e.g., an Advanced Instrument Inc 2020 freezing point depression osmometer).

The pharmaceutical compositions described herein can also have various pH levels. The pH of the pharmaceutical composition can be adjusted by any method known in the art, such as, for example, addition of a buffer.

In some embodiments, the pharmaceutical composition includes an anti-Cx43 antibody described herein, a histidine/histidine hydrochloride buffer, Polysorbate 80, and sucrose. In some embodiments, the pharmaceutical composition includes about 40 mg/mL to about 60 mg/mL of an anti-Cx43 antibody described herein; about 10 mM to about 40 mM histidine/histidine hydrochloride buffer; about 0.005% w/v to about 0.05% w/v Polysorbate 80; and about 1% w/v to about 20% w/v sucrose. In some embodiments, the pharmaceutical composition includes about 50 mg/mL of an anti-Cx43 antibody described herein; about 20 mM histidine/aspartic acid buffer; about 0.02% w/v Polysorbate 80; and about 8% w/v sucrose. In some embodiments, the pharmaceutical composition has a pH of between about 5.4 to about 5.6. In some embodiments, the pharmaceutical composition has a pH of about 5.5.

It may be advantageous to formulate parenteral compositions or unit dosage form for ease of administration and uniformity of dosage. Unit dosage form as used herein refers to physically discrete units suited as unitary dosages for the patients to be treated: each unit contains a predetermined quantity of active agent calculated to produce the desired therapeutic effect in association with any required pharmaceutical carrier. The specification for unit dosage forms is dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitations, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

The formulations described herein are administered to a subject in need of treatment with the anti-Cx43 antibodies, preferably a human, in accord with known methods, such as intravenous administration as a bolus or by continuous infusion over a period of time, by intramuscular, intradermal, intraperitoneal, intracerobrospinal, subcutaneous, intra-articular, intrasynovial, intrathecal, oral, topical, or inhalation routes.

In some embodiments, the anti-Cx43 antibodies are administered to a subject by intravenous or subcutaneous (i.e., beneath the skin) administration. For such purposes, the formulation may be injected using a syringe. However, other devices for administration of the formulation are available such as injection devices (e.g., the INJECT-EASE™ and GENJECT™ devices); injector pens (such as the GENPEN™); auto-injector devices, needleless devices (e.g., MEDIJECTOR™ and BIOJECTOR™); and subcutaneous patch delivery systems.

In a specific embodiment, the present disclosure is directed to kits for a single dose-administration unit. Such kits comprise a container of an aqueous formulation of therapeutic protein or antibody, including both single or multi-chambered pre-filled syringes. Exemplary pre-filled syringes are available from Vetter GmbH, Ravensburg, Germany.

### III. Method of Treating Spinal Cord Injury

Certain aspects of the present disclosure provide methods of treating spinal cord injury in a subject. The methods comprise blocking the opening of Cx43 hemichannels in astrocytes in a subject in need thereof by using, e.g., an anti-Cx43 antibody.

Spinal cord injury is defined as damage to the spinal cord that causes temporary or permanent changes in its function, and this condition has a high incidence, high costs, a high disability rate and a low age of onset. Serious SCI represents a significant physical, psychological and financial burden for patients and their families. According to the cause of injury, SCI can be divided into traumatic and nontraumatic SCI. According to the pathophysiology, acute SCI can be divided into primary and secondary injuries. According to the severity, SCI can be divided into complete or incomplete injury, and incomplete SCI can manifest as central core syndrome, Brown-Sequard syndrome, anterior cord syndrome, and posterior core syndrome.

The pathophysiology of SCI includes primary injury and secondary injury; the former is usually a mechanical injury to the cord, and the latter is the consequence of cell and biological reactions to the primary injury, which involve the immune system, nervous system, vascular system, etc., including hemorrhage, ischemia, oxidative stress, inflammatory reaction, neural cell death, demyelination, scar formation and so on. Studies have shown that the mechanism of nerve regeneration in the zebrafish SCI model may be related to extracellular matrix Cthrc1 or pro-regenerative macrophages. In a study on the pathological mechanism of the model of spinal cord hemisection injury in nonhuman primates, it reported that activated microglia/macrophages were found both within the injury center and the peri lesion area, and in contrast to rodent, substantial reactive astrocytic responses at the lesion border were not observed in the monkey. Conversely, a deposit of robust fibrotic scar was observed at the injury epicenter, which filled the space originally created by the hemisection.

The recovery of spinal cord function depends on the remodeling and integrity of neural circuits. After SCI, the breakage of neuronal axons and the death of neurons cause dysfunction of neural circuits. The plasticity of neural circuits is the basis of the recovery of neural function. The traditional repair principle is to promote the regeneration and extension of the corticospinal tract (CST) and reestablish the connection with distal neurons, including reducing the production of regenerative-related inhibitors, such as chondroitin sulfate proteoglycans (CSPG)/NogoA/myelin-associated glycoprotein (MAP)/oligodendrocyte myelin glycoprotein (OMG), and even lipid metabolites, in the microenvironment during the early stage of SCI or promoting axon regeneration by exploiting the intrinsic growth ability. Phosphatase and tensin homolog (PTEN) deletion effectively enhanced the regenerative ability of adult corticospinal neurons and promoted the recovery of motor function after SCI. However, regenerated axons can hardly be reconnected to distal effectors because of the long distance.

At present, the clinical treatment methods for SCI include medications, surgery, rehabilitation and nursing. Management in the acute phase aims mainly to stabilize the condition and ensure the survival of patients. Management in the chronic phase aims mainly to restore function, reduce complications, and encourage patients to return to society and work. During this period, it may also be necessary for psychologists to treat patients with psychological disorders. Breakthroughs in clinical treatment have mainly focused on the research and development of new drugs, clinical trials of cell therapies and biomaterial transplantation, new physical regulation approaches, artificial intelligence, etc.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells, and they mediate intercellular communication. Hemichannels are a separate entity from gap junction channels. Hemichannels permit the exchange of molecules between the intracellular compartments and the extracellular environment.

Cx43 is also known as gap junction alpha-1 protein (GJA1), which is a 43.0 kDa protein composed of 382 amino acids (NCBI Reference Sequence: NP 000156.1). GJA1 contains a long C-terminal tail, an N-terminal domain, and multiple transmembrane domains. The protein passes through the phospholipid bilayer four times, leaving its C- and N-terminals exposed to the cytoplasm. The C-terminal tail is composed of 50 amino acids and includes post-translational modification sites, as well as binding sites for transcription factors, cytoskeleton elements, and other proteins. As a result, the C-terminal tail is central to functions such as regulating pH gating and channel assembly. Notably, the DNA region of the GJA1 gene (NCBI Gene ID: 2697) encoding this tail is highly conserved, indicating that it is either resistant to mutations or becomes lethal when mutated. Meanwhile, the N terminal domain is involved in channel gating and oligomerization and, thus, may control the switch between the channel's open and closed states. The transmembrane domains form the gap junction channel while the extracellular loops facilitate proper channel docking. Moreover, two extracellular loops form disulfide bonds that interact with two hexamers to form a complete gap junction channel.

ALMB-0166 is a novel humanized monoclonal IgG1 antibody that inhibits connexin 43 (Cx43) hemichannels in spinal cord astrocytes, thereby reducing astrocyte/microglial activation after acute spinal cord injury. Inhibition of astrocyte Cx43 hemichannels can reduce the release of ATP, glutamate, ions, and other molecules that cause excitation toxicity, inflammation, and metabolic stress. Therefore, ALMB-0166 has the potential to inhibit secondary injury after spinal cord injury, thereby promoting the recovery of functions such as the nervous system and motor (O'Carroll et al., Neurosci Res, 75(3): 256-267 (2013); O'Carroll et al., Methods Mol Biol; 1037: 519-546 (2013)).

“Treatment” refers to therapeutic treatment. Those in need of treatment include those already with disease. Hence, the subject, e.g., a human, to be treated herein may have been diagnosed as suffering from a disease, such as spinal cord injury. A disease, e.g., SCI, is “inhibited” or “treated” if at least one symptom (as determined by responsiveness/non-responsiveness, or indicators known in the art and described herein) of the condition is alleviated, terminated, slowed, minimized, or prevented. The terms “patient” and “subject” are used interchangeably herein.

The term “subject” or “patient” refers to either a human or non-human, such as primates, mammals, and vertebrates. In particular embodiments, the subject is a human.

Treatment can be suitably administered to subjects, particularly humans, suffering from, having, susceptible to, or at risk of developing spinal cord injury. Determination of those subjects “at risk” can be made by any objective or subjective determination by a diagnostic test or opinion of a subject or health care provider. Identifying a subject in need of such treatment can be in the judgment of a subject or a health care professional and can be subjective (e.g., opinion) or objective (e.g., measurable by a test or diagnostic method).

The anti-Cx43 antibody for use in any of the methods disclosed herein can be stored as a lyophilized solid or an aqueous formulation, or any other forms known in the art. In the case of an anti-Cx43 antibody which is stored as a lyophilized solid, the antibody is reconstituted in a solution such as water (e.g., for injection) prior to administration. If prepared for infusion either from a lyophilized form or an aqueous formulation, the final concentration, e.g., after dilution of the reconstituted antibody (e.g., in a saline, Ringer's or 5% dextrose infusion system) of the anti-Cx43 antibody can be about 0.1 mg/ml to about 80 mg/ml for administration. The final concentration may be about 0.1 mg/ml to about 80 mg/ml, about 0.5 mg/ml to about 70 mg/ml, about 1 mg/ml to about 60 mg/ml, about 5 mg/ml to about 50 mg/ml, about 10 mg/ml to about 40 mg/ml, about 15 mg/ml to about 30 mg/ml, or about 20 mg/ml to about 25 mg/ml. In some embodiments, the final dosage form may be at a concentration of about 0.1 mg/ml, about 0.5 mg/ml, about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml or higher than 80 mg/ml.

The term “effective amount,” as used herein, refers to that amount of an agent, such as an anti-Cx43 antibody, which is sufficient to effect treatment, prognosis or diagnosis of SCI, when administered to a patient or a subject. Dosage regimens may be adjusted to provide the optimum therapeutic response. An effective amount is also one in which any toxic or detrimental effects (side effects) of the agent are minimized and/or outweighed by the beneficial effects. A therapeutically effective amount will vary depending upon the patient and disease condition being treated, the weight and age of the patient, the severity of the disease condition, the manner of administration, course of the condition, patient's clinical history and response to anti-Cx43 antibody and the like, which can readily be determined by one of ordinary skill in the art. For example, an effective amount or a dose of the anti-Cx43 antibody ranges from about 0.01 mg/kg to about 1000 mg/kg. In some embodiments, the effective amount or the dose of the anti-Cx43 antibody is about 0.01 mg/kg to about 900 mg/kg, about 0.1 mg/kg to about 800 mg/kg, about 0.5 mg/kg to about 700 mg/kg, about 1 mg/kg to about 600 mg/kg, about 1.5 mg/kg to about 500 mg/kg, about 2 mg/kg to about 400 mg/kg, about 5 mg/kg to about 300 mg/kg, about 10 mg/kg to about 200 mg/kg, about 15 mg/kg to about 100 mg/kg, about 20 mg/kg to about 50 mg/kg, about 25 mg/kg to about 45 mg/kg, or about 30 mg/kg to about 40 mg/kg. In some embodiments, the effective amount or the dose of the anti-Cx43 antibody is about 0.01 mg/kg, about 0.1 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, or more than 1000 mg/kg.

In specific embodiments, the anti-Cx43 antibody is administered at a dosage of 0.01 mg/kg to 100 mg/kg. In some embodiments, the anti-Cx43 antibody is administered at a dosage of 15 mg/kg. In some embodiments, the anti-Cx43 antibody is administered at a dosage of 25 mg/kg. In some embodiments, the anti-Cx43 antibody is administered at a dosage of 50 mg/kg.

In some embodiments, the anti-Cx43 antibody is administered at a dosage from 100 mg to 6000 mg. In some embodiments, the dosage of the anti-Cx43 antibody is 200 mg to 4800 mg, 300 mg to 4000 mg, 400 mg to 3500 mg, 500 mg to 3000 mg, 600 mg to 2400 mg, 800 mg to 2000 mg, or 1000 mg to 1600 mg, In some embodiments, the anti-Cx43 antibody is administered at a dosage of less than 100 mg, about 100 mg, about 200 mg, about 300 mg, about 400 mg, about 500 mg, about 600 mg, about 800 mg, about 1200 mg, about 1600 mg, about 2000 mg, about 2400 mg, about 2800 mg, about 3200 mg, about 3600 mg, about 4000 mg, about 4400 mg, about 4800 mg, about 5000 mg, about 5500 mg, about 6000 mg, or about 7000 mg.

In the methods as described herein, the anti-Cx43 antibody is administered about once every day, about once every 2 days, about once every 3 days, about once every 4 days, about once every 5 days, about once every 6 days, about once every week, about once every 8 days, about once every 9 days, about once every 10 days, about once every 11 days, about once every 12 days, about once every 13 days, about once every 2 weeks, about once every 15 days, about once every 16 days, about once every 17 days, about once every 18 days, about once every 19 days, about once every 20 days, about once every 3 weeks, about once every 22 days, about once every 23 days, about once every 24 days, about once every 25 days, about once every 26 days, about once every 27 days, about once every 4 weeks, about once every 29 days, about once every 30 days, about once every 31 days, about once every 32 days, about once every 33 days, about once every 34 days, about once every 5 weeks, about once every 36 days, about once every 37 days, about once every 38 days, about once every 39 days, about once every 40 days, or about once every 41 days, about once every 6 weeks, about once every 7 weeks, about once every 8 weeks, about once every 9 weeks, about once every 10 weeks, about once every 11 weeks, about once every 12 weeks, about once every 13 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks or 6 months, or about once every more than 24 weeks or 6 months.

In some specific embodiments, the anti-Cx43 antibody is administered about once every week. In one particular embodiment, a dose of the anti-Cx43 antibody is administered once every week. In another particular embodiment, a dose of the anti-Cx43 antibody is administered once every week for 1, 2, 3, 4, 5, 6, 7, 8, 9, 10, or longer than 10 weeks.

An effective amount of the anti-Cx43 antibody is suitably administered to the patient at one time or over a series of treatments and may be administered to the patient at any time from diagnosis onwards. The anti-Cx43 antibody may be administered as the sole treatment or in conjunction with other drugs or therapies useful in treating SCI.

In the methods as described herein, an effective amount or a dose of the anti-Cx43 antibody can be administered to a patient over less than 5 minutes, about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more than 180 minutes. In some specific embodiments, the effective amount or the dose of the anti-Cx43 antibody can be administered to a patient over about 30 minutes. In one specific embodiment, the effective amount or the dose of the anti-Cx43 antibody can be intravenously administered to a patient over 60 minutes.

In the methods described herein, an anti-Cx43 antibody is administered to a patient. If the anti-Cx43 antibody is in a formulation which is in a solid, e.g., dry state, the process of administration can comprise a step of converting the formulation to a liquid state. In one aspect, a dry formulation can be reconstituted, e.g., by a liquid as described above, for use in injection, e.g. intravenous, intradermal, intramuscular, intraperitoneal or subcutaneous injection. In another aspect, a solid or dry formulation can be administered topically, e.g., in a patch, cream, aerosol or suppository.

The patient or subject, before, during, or after being treated with the anti-Cx43 antibody, can be examined for evaluation indicators of SCI using technologies and methods known in the art. Non-limiting examples of the common medical technologies and methods used to examine and diagnose SCI include: physical examination by a physician, laboratory tests etc. The choice of technologies and methods, and the frequency of examination can be determined and/or adjusted by a person skilled in the art based on the patient's or subject's specific conditions. In some embodiments, at least one evaluation indicator of SCI is assessed at least one day to one week after the anti-Cx43 antibody is administered to the subject. In some embodiments, at least one evaluation indicator of SCI is assessed a day, two days, three days, four days, five days, six days, or a week after the anti-Cx43 antibody is administered to the subject.

In some embodiments, at least one evaluation indicator of SCI is improved after the administration of the anti-Cx43 antibody. Non-limiting examples of the evaluation indicators of SCI include: ASIA total sensory score, ASIA total sensory score of the left body, ASIA total sensory score of the right body, total pinprick score, total light touch score, total motor score, total motor score of the left body, total motor score of the right body, upper limb motor score, lower limb motor score, injury grading, and pain visual analogue scale.

In some embodiments, the anti-Cx43 antibody inhibits secondary SCI.

In some embodiments, the subject treated with the anti-Cx43 antibody for SCI has a duration of response of at least 5 days, at least 10 days, at least 15 days, at least 20 days, at least 25 days, at least 1 month, at least 2 months, at least 3 months, or more.

In some embodiments, the subject treated with the anti-Cx43 antibody for SCI has a time to response less than 3 months, less than 2 months, less than 1 months, less than 25 days, less than 20 days, less than 15 days, less than 10 days, less than 7 days, less than 6 days, less than 5 days, less than 4 days, less than 3 days, less than 2 days, or less than 1 day.

As used herein, a “response” or “being responsive” to a treatment refers to the subject having improvement of at least one parameter of disease progression. The subject can have partial response or complete response to a treatment. The response to a treatment can be determined based on methods known in the art. A person skilled in the art can determine the proper methods based on the type of diseases being evaluated.

### EXAMPLES

### Example 1. Safety, Tolerability and Efficacy of ALMB-0166 in the Patients with Acute Spine Cord Injury (SCI): A Multicenter, Randomized, Placebo-Controlled, Phase I/II Study

### I. Study Plan

### A. Overall Study Design and Plan

This study was a multi-center, randomized, double-blind, placebo-controlled, single-dose, dose-escalation Phase I clinical study to evaluate the safety, tolerability, and PK characteristics of ALMB-0166 in the treatment of patients with acute spinal cord injury.

The single ascending dose study included three dose groups: 200 mg (or 3 mg/kg), 600 mg, and 1200 mg. Among them, each of the 200 mg and 600 mg dose groups enrolled 6 subjects, who were randomly assigned to the ALMB-0166 group and control group at a ratio of 2:1; each of the 1200 mg and other unplanned dose groups enrolled 4 subjects, who were randomly assigned to the ALMB-0166 group and control group at a ratio of 3:1. The subjects in the ALMB-0166 group and the control group received intravenous drip infusion of the ALMB-0166 product or placebo, and at the same time, all subjects received the best supportive care.

Each subject was closely monitored for 72 hours after dosing and followed up on days 7, 14, 28, and 56 post-dose. If a subject experienced any AE, the investigator would determine whether an unscheduled visit or an extension of the visit duration is necessary. After dosing, all subjects in each dose cohort should complete at least 28 days of observation, and when all safety data and PK data (including safety laboratory test data) are available, the project statistician would provide relevant information of the subjects in the current dose cohort to the SRC members; if the conditions for unblinding were met (see SRC charter for details), the unblinded statistician would unblind the subjects in the current dose cohort and pass the blind code to the SRC members. The SRC members would then evaluate all available safety data for this dose cohort based on the safety data to decide whether to proceed to the next dose cohort, or whether to add other unplanned dose cohorts. Dose groups that had completed safety evaluation might be expanded to up to 8 subjects upon assessment by the investigators and the sponsor based on prior clinical data (excluding dropouts). Expanded subjects would receive ALMB-0166 or placebo according to the principles of randomization, double-blind, and placebo control (ALMB-0166 group:control group=3:1).

If the dose escalation reached the preset highest dose group and the safety and tolerability of that dose group remain good, then the SRC might jointly discuss and decide whether to attempt higher doses. The final administered doses were 3 mg/kg or 200 mg, 600 mg, 1200 mg, 2400 mg, 4800 mg.

TABLE 1
Dose-escalation Setting of Single Dose

| Dose Group | Dose (mg) | Sample Size* |
|---|---|---|
| 1 | 200 (or 3 mg/kg) | 6 |
| 2 | 600 | 6 |
| 3 | 1200 | 4 |
| Other Doses | 1200/2400^{#} | 4/4 |

*Dose groups that have completed safety evaluation may be expanded to up to 8 subjects upon assessment by the investigators and the sponsor based on prior clinical data (excluding dropouts).
^{#}If the dose escalation reaches the preset highest dose group and the safety and tolerability of that dose group remain good, then the SRC may jointly discuss and decide whether to attempt higher doses.

### B. Discussion of Study Design

### 1. Scientific Rationale of the Study Design

The scientific rationale of this study design included a few following aspects: (1) the safety and tolerability evaluations selected currently recognized and commonly used evaluation indicators such as physical examination, vital signs, safety laboratory tests, electrocardiogram, and the occurrence of AE and SAE; (2) the pharmacokinetic evaluation selected currently recognized and commonly used evaluation indicators such as AUC₀₋ₜ, AUC_{0-inf}, Cₘₐₓ, Tₘₐₓ, t_{1/2}, and CL/F; (3) the efficacy evaluation selected the currently recognized and commonly used evaluation indicators for acute spinal cord injury: the ASIA score and the visual analog scale; and (4) the immunogenicity evaluation selected the currently recognized and commonly used evaluation indicator ADA.

### 2. Rationale for Dose Selection

a. Estimation of Starting Dose

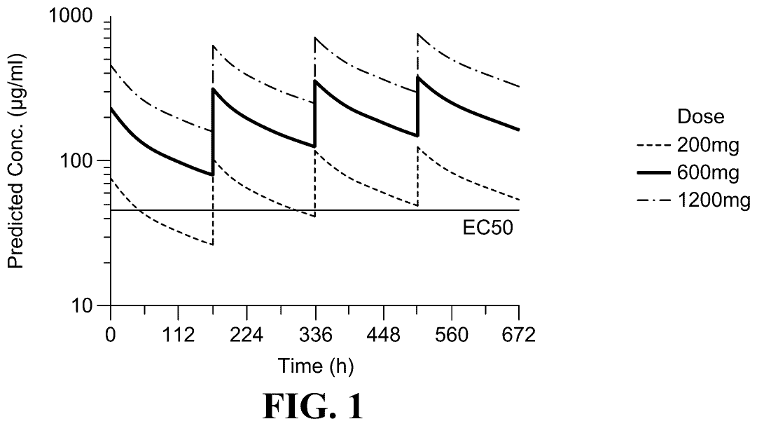

The in vitro pharmacodynamic results showed that in human astrocytes, the antibody concentration of ALMB-0166 causing 50% of the maximal binding effect (EC₅₀) was 46.00±2.27 g/mL; the EC₅₀ for inhibiting the opening of Cx43 hemichannels (in the presence of pro-inflammatory factors IL-1β/TNFα) was 10.56±0.12 μg/mL. In vivo pharmacodynamic tests showed that at a dose level of 25 mg/kg, ALMB-0166 could significantly inhibit the opening of spinal cord Cx43 hemichannels near the injury site in a mouse model of acute spinal cord injury, and behavioral tests demonstrated that it could improve limb function after injury from acute spinal cord injury. At this dosage, the exposure data AUCinf of ALMB-0166 in healthy mice was approximately 36 h·mg/mL. Based on existing pharmacokinetic data from healthy human subjects in Australia, a fixed dose of ALMB-0166 at 600 mg administered once a week achieved the exposure level after predicted steady state under the efficacious dose in animals, and the blood concentration is always maintained above the EC₅₀ in the in vitro studies (see FIG. 1).

Preclinical animal tissue distribution results indicated that ALMB-0166 distributed into the spinal cord of animals with acute spinal cord injury, while no distribution was detected in tissues with high expression of Cx43 in healthy animals. Therefore, estimating the human equivalent dose based on exposure data from healthy mice and healthy human circulation had deviations and could not fully represent the drug exposure in the spinal cord. For safety reasons, a recommended starting dose of 200 mg for a single administration in patients was proposed.

The mouse acute spinal cord injury model was a disease-related animal model for efficacy exploration of this product. In this experiment, model mice received a maximum intravenous dose of 50 mg/kg, and no animal deaths or significant clinical observations occurred, indicating that the NOAEL dose was greater than 50 mg/kg. Therefore, based on the exposure (AUC) in mice and humans, the estimated human equivalent dose was greater than 18 mg/kg (assuming a human body weight of 60 kg, fixed dose of 1080 mg). Therefore, it was considered that the setting of a starting dose of 200 mg in the ALMB-0166-CN-101 protocol was controllable from a safety perspective.

The 4-week toxicology study data in cynomolgus monkeys indicated that intravenous administration of ALMB-0166 at a dose of 250 mg/kg weekly did not observe any toxicity reactions related to ALMB-0166. At this dose, the serum AUC was approximately 370 h·mg/mL, which was much higher than the exposure at 200 mg in humans (see Table 2).

b. Determination of the Maximum Incremental Dose

The maximum dose in this study was provisionally set at 1200 mg. Firstly, based on the PK data obtained from healthy subjects in Australia, it was predicted that when ALMB-0166 was administered at a fixed dose of 1200 mg once weekly, the steady-state Cₘₐₓ and exposure were still lower than those in mice (the NOAEL dose in mice with acute spinal cord injury model was greater than 50 mg/kg, referencing the PK data after administering 50 mg/kg to healthy mice) and the exposure at the NOAEL level in monkeys (see Table 2 for details). In addition, in the single-dose escalation phase of the ongoing ALMB-0166-AU-101 study, after intravenous drip infusion of ALMB-0166 at 3 mg/kg, 10 mg/kg, or 25 mg/kg in healthy subjects, no SAEs occurred, and tolerability was good. Simultaneously, this study will adjust the administered dose based on the PK and safety data obtained from Chinese patients, if necessary. If the dose escalation reaches the preset highest dose group and the safety and tolerability of that dose group remain good, then the SRC may jointly discuss and decide whether to attempt higher doses.

TABLE 2
Predicted Human Exposure Data after Single Administration at Different Dose
Groups vs. Exposure after Repeated Dosing at NOAEL Level in Monkey.

| Dose | C_{max, H} | C_{max, MO} | | C_{max, ss, M} | | AUC_{0-168, H} | AUC_{tau, MO} | | AUC_{tau, M} | |
|---|---|---|---|---|---|---|---|---|---|---|
| (mg) | (μg/mL) | (μg/mL) | SF_{H/M} | (μg/mL) | SF_{H/M} | (h · mg/mL) | (h · mg/mL) | SF_{H/M} | (h · mg/mL) | SF_{H/M} |
| 200 | 76.2 | 1366 | 17.9 | 5743 | 75.4 | 6.82 | 76 | 11.1 | 373 | 54.7 |
| 600 | 229 | | 6.0 | | 25.1 | 20.5 | | 3.7 | | 18.2 |
| 1200 | 457 | | 3.0 | | 12.6 | 40.9 | | 1.9 | | 9.1 |

Note:
C_{max, H}: Predicted peak concentration after single dose administration in humans; C_{max, ss, MO/M}: Peak concentration after the fourth weekly administration in mouse (MO)/monkey (M) repeated toxicity studies at dose groups of 50 mg/kg\250 mg/kg; AUC_{0-168, H}: Predicted area under the concentration-time curve after single dose administration in human; AUC_{tau, MO/M}: Area under the concentration-time curve after the fourth weekly administration in mouse/monkey repeated toxicity studies at dose groups of 50 mg/kg\250 mg/kg.

Based on the PK data from single doses of 1 mg/kg to 25 mg/kg in healthy Australians, multiple doses of 25 mg/kg sequentially decreased to 12.5 mg/kg, and doses of 200 mg, 600 mg, and 1200 mg in Chinese patients with acute spinal cord injury, a PopPK model was established to predict exposures at higher dose groups. Analysis of preclinical efficacy data suggests that the potential effective dose was approximately at 2400 mg and higher dose groups. In terms of safety, the Cₘₐₓ and AUC at doses of 2400 mg, 4800 mg, and 6000 mg were approximately 14% and 40%, 28% and 60%, 36% and 101% of the exposures at NOAEL levels in crab-eating monkey toxicity studies. Considering a comprehensive evaluation of safety and efficacy, subsequent dose escalation added 2400 mg and 4800 mg to further explore the safety and efficacy of ALMB-0166.

c. Determination of the Maximum Incremental Dose

A total of 3 dose groups were designed: 200 mg (or 3 mg/kg), 600 mg, and 1200 mg, administered once.

After the safety data of each dose group were summarized and evaluated by the SRC, the SRC determined whether to proceed to the next dose group or decided whether to add other unplanned dose groups. Dose groups that had completed safety evaluation might be expanded to up to 8 subjects upon assessment by the investigators and the sponsor based on prior clinical data (excluding dropouts). If the dose escalation reached the preset highest dose group and the safety and tolerability of that dose group remain good, then the SRC might jointly discuss and decide whether to attempt higher doses. The final dose escalation included a total of 5 dose groups: 200 mg (3 mg/kg), 600 mg, 1200 mg, 2400 mg, and 4800 mg.

### C. Study Interventions and Concomitant Medications

### 1. Study Intervention Description

ALMB-0166 is an anti-human Cx43 hemichannel monoclonal antibody, which is a sterile, non-hot intravenous administration injection; the control was a placebo, which is the blank solvent of the ALMB-0166 injection, only without the active ingredient. Information on ALMB-0166 and the control are shown in Table 3, both provided by AlaMab Therapeutics Inc.

TABLE 3
Information on ALMB-0166 and Placebo.

| | ALMB-0166 | Placebo |
|---|---|---|
| Active | Monoclonal Antibody Against Human Cx43 | Inactive Ingredients |
| Ingredient | Hemichannels | |
| Strength | 150 mg (3 mL)/vial | 3 mL/vial |
| Dose | 200 mg (or 3 mg/kg), 600 mg, 1200 | None |
| | mg, 2400 mg, 4800 mg | |
| Route of | Intravenous infusion, drip infusion | Intravenous infusion, drip infusion |
| Administration | within 60 ± 5 min | within 60 ± 5 min |
| Batch No. | 201904047 | 20210501 |
| Storage | Store at −20° C. ± 5° C., protect from | Store at −20° C. ± 5° C., protect from |
| Method | light befor euse, and avoid shaking | light before use, and avoid shaking |

### 2. Subject Allocation and Randomization

The single ascending dose study included three dose groups: 200 mg (or 3 mg/kg), 600 mg, and 1200 mg. Among them, each of the 200 mg and 600 mg dose groups enrolled 6 subjects, who were randomly assigned to the ALMB-0166 group and control group at a ratio of 2:1; each of the 1200 mg and other unplanned dose groups enrolled 4 subjects, who were randomly assigned to the ALMB-0166 group and control group at a ratio of 3:1. The final dose escalation included a total of 5 dose groups: 200 mg (or 3 mg/kg), 600 mg, 1200 mg, 2400 mg, and 4800 mg.

This study used block randomization by dose group, with competitive enrollment across sites. The order in which each subject entered the ALMB-0166 group or control group in the study was randomly determined. The randomization code list was generated by an independent unblinded statistician using SAS (version 9.4 or higher), where the 200 mg and 600 mg dose groups each enrolled 6 subjects, randomly generated according to a 2:1 ratio using the block randomization method; the 1200 mg and other unplanned dose groups were randomly generated according to a 3:1 ratio using the block randomization method. IWRS was used; the investigators, subjects, sponsor's blinded personnel, and PK testing personnel were completely unaware of the study grouping; the SRC would hold discussions under blind and make decisions on dose escalation.

After the subjects passed the screening process, the researchers 1 logged in to the IWRS (Interactive Web Response System) to request a randomization number and print or download and save the randomization information from the IWRS. Sites were numbered starting from 01, for example, the first site was numbered 01. During screening, each subject was identified by a screening number, expressed as S+site number+three-digit Arabic numeral, for example, SO1001. Eligible subjects were randomized and assigned a randomization number prior to the first dose. Randomization numbers were represented by four-digit Arabic numerals, such as: 1101.

For any reason, regardless of whether ALMB-0166 was used, randomized subjects who had withdrawn or been withdrawn from the clinical study, or subjects who had been inadvertently unblinded or urgently unblinded, retained their randomization numbers, which could not be reassigned to other subjects for reuse.

### 3. Blinding

This clinical study is a double-blind study; all subjects and all sponsors and investigators involved in therapeutic clinical assessments were unaware of who was receiving what kind of treatment, including those selecting eligible subjects, outcome evaluators, or those evaluating compliance according to the protocol; that is, clinical investigators, project managers, project monitors, data management, and statistical analysis personnel were all set cegity.

a. Blinded Design

The ALMB-0166 or placebo used by each subject was packaged in sealed boxes with identical appearance.

b. Blinding and Storage of Blind Codes

An independent unblinded statistician, who was not involved in data management or statistical analysis of this study, used SAS 9.4 statistical software on a computer to generate the list of drug numbers and their corresponding treatment groups, and generated the code list of drug numbers using the block randomization method.

The block lengths selected during the generation of the lists of subject randomization numbers and drug code numbers, along with the random initial seed parameters, were sealed together as confidential data in the blinding documentation.

The on-site blinding of ALMB-0166 was conducted by an unblinded independent statistician and personnel unrelated to this study from the sponsor site. The unblinded statistician, based on the drug numbers generated by the software and their corresponding groups (ALMB-0166 group or control group), guided the blinded personnel to affix labels containing the drug numbers onto the smallest packaging of ALMB-0166. This study used a central randomization system to dispense ALMB-0166 according to the visit schedule. All ALMB-0166 and placebo were dispensed in their smallest packaging units.

c. Emergency Letter

An emergency letter (electronic) was prepared for each randomization number. The electronic emergency letter recorded the subject's randomization number and the group (ALMB-0166 group or control group) corresponding to the drug number received by the subject. Electronic emergency letters were provided for emergency unblinding use; strictly authorized operations were only authorized to the director of each site. Only the director of each site may apply to open the electronic emergency letter. The operation trail retained the operator's operational traces, date, and information such as the reason for opening.

d. Unblinding Provisions

Interim Unblinding: After all subjects in each dose group completed at least 28 days of observation post-dosing and all safety data and PK data (including safety laboratory test data) were available, if the unsecity conditions are met (see SRC charter), the project manager (secity) will fill out the “Blinding Code Application” to unblind the current dose group, and after being signed and approved by the principal investigator, it was sent to the unblinded statistician. The unblinded statistician emailed the blinding code to designated personnel, and the recipients of the blinding code should properly manage the blinding code.

End-of-study Unblinding: After all study data were fully verified and locked, unblinding was carried out by the principal investigator and the sponsor, both needed to sign the “Blinding Code Application”, sent it to the unblinded statistician. The unblinded statistician emailed the blinding code to the designated personnel.

e. Emergency Unblinding:

In emergency situations, when the investigator believed that knowing the medication used by the subject is beneficial for the management of adverse events, an application for emergency unblinding could be made. Within 24 hours after emergency unblinding, notify the clinical study responsible (leading) institution, the clinical monitor at this site, and sponsor personnel such as statistical personnel, and explain the reason for unblinding.

Unblinding cases were comprehensively considered by the investigator to decide whether to withdraw; that was, cases were not directly dropped due to unblinding, and the data of unblinding cases should be kept complete. Because the testing institution is independent and does not have access to the blinding code, the pharmacokinetic data of the study cases after unblinding were still included in the analysis.

### 4. Study Intervention

a. Dose Escalation, Dose Adjustment, Dose Termination, and Addition of Extra Doses of Study Intervention

Administration of ALMB-0166 or placebo was completed by drip infusion within 60±5 min. ALMB-0166 and placebo were administered via intravenous infusion. ALMB-0166 or placebo was diluted with sterile normal saline (0.9% NaCl) to concentrations of 0.5 mg/mL to 10 mg/mL; for detailed procedures, refer to the drug preparation operation manual.

Dose escalation, dose adjustment, dose termination, and addition of extra doses were decided by the SRC through consultation. The SRC consists of representatives from the investigators and the sponsor. After the subjects in each dose group were observed for at least 28 days post-dosing (excluding an additional 3 subjects that may be expanded in each dose group) and all safety data and PK data (including safety laboratory test data) were available, the project statistician provided relevant information of subjects in the current dose group to SRC members; if the unblinding conditions are met (see SRC charter for details), the unblinded statistician unblind the subjects in the current dose group and passes the blinding code to SRC members, who summarize the safety data of that dose group according to the blinding code. The SRC holds a meeting to review these safety data and makes the following decisions:
1. Proceeding to the next planned dose level escalation;
2. Adjustment for dose escalation protocol;
3. Decisions on whether to add unplanned dose escalation and the unplanned dose;
4. Recommendations for stopping dose escalation, pausing, and terminating the study; Individual case assessment of safety and tolerability.

### 5. Concomitant Therapies

Alcohol use was prohibited from after the occurrence of spinal cord injury until the intensive PK sample collection period (within D14±1). Use of gangliosides was prohibited during the study. During the study, any treatments for acute spinal cord injury and its related complications or other diseases (excluding prohibited medications) were allowed, but it was necessary to closely observe whether the treatment and medications used affect the efficacy or safety of ALMB-0166.

### 6. Interruption/Discontinuation of Study Interventions and Subject Withdrawal

a. Interruption Discontinuation of Study Interventions

Discontinuation of study intervention is defined as the investigator confirming that the subject needs to undergo treatment withdrawal for any reason. Discontinuation of study intervention does not imply study termination; subsequent study procedures should be completed according to the protocol. If the subject experienced a clinically significant change after enrollment (including but not limited to change from baseline), the investigator or qualified designee would decide whether any change in subject management is required.

Data to be collected when the study intervention was discontinued included: safety data (adverse events, laboratory tests, vital signs, electrocardiogram, imaging examinations); efficacy data (ASIA sensory and motor scores, impairment grading).

b. Subject Withdrawal

Subjects might withdraw from the study at any time.

The investigator might decide to discontinue or withdraw a subject from the study for the following reasons:
Pregnancy;
Poor compliance with the study treatment;
In the event of clinical AEs, laboratory abnormalities, or other medical conditions, continued participation in the study will not be in the best interest of the subject;
The subject meets exclusion criteria (newly emerged or previously unobserved) and cannot continue to participate in the study;
Other discontinuations determined by the investigator.

The reason for the subject's discontinuation of the study or withdrawal from the study should be recorded in the electronic case report form.

Subjects who have signed the ICF and been randomized but have not received the study intervention treatment or who withdraw from the study within 14 days after the first administration may be replaced. For subjects who have signed the ICF and been randomized, have received the study intervention treatment and been observed for more than 14 days but subsequently discontinue the study, replacement is not allowed.

c. Lost to Follow-Up

If a subject failed to return to the study site for a scheduled visit and the study site personnel was unable to establish contact, the subject would be classified as lost to follow-up.

If a subject failed to return to the study site for the scheduled study visit, the subsequent actions must be implemented:
(1) The study site tried to contact the subject to reschedule any missed visits, explained the significance of adhering to the visit schedule, and ascertained whether the subject was willing to and/or should continue in the study.
(2) Before a subject was considered lost to follow-up, the investigator or designee would make every effort to re-contact the subject (if possible, make more than three calls to the subject or his/her family member's mobile phone number, fixed phone number, WeChat number, etc. at different time intervals. WeChat and SMS messages might be sent, or a registered letter may be sent to the subject's latest mailing address, or contact the subject via a locally valid contact method, as appropriate). Attempts to contact subjects should be recorded in the subject's medical records or study files.
(3) If the subject remained unavailable, he/she would be considered withdrawn from the study due to lost to follow-up.
   d. Study Termination

This study might be temporarily suspended or prematurely terminated if there was sufficient reasonable cause. The party suspending or terminating the study should provide written notice to subject, investigator, sponsor, and regulatory authority and document the reason for the suspension or termination of the study. If the study was prematurely terminated or temporarily discontinued, the Principal Investigator should promptly inform the subject, the Ethics Committee, and the sponsor, and provided reasons for the study termination or temporary discontinuation. The investigator would contact the subject and notify the change in the scheduled visit time.

Conditions that might warrant study termination or temporary discontinuation include but were not limited to:
(1) The study brings definite unexpected, major or unacceptable risks to subjects
(2) Efficacy being confirmed to terminate the study
(3) Poor compliance with protocol requirements
(4) Incomplete and/or non-evaluable data
(5) The primary endpoint having been reached
(6) The study treatment confirmed to be ineffective

The study may continue only if issues related to safety, protocol compliance, and data quality are resolved and the requirements of the sponsor, ethics committee, and/or regulatory authorities are met.

### D. Clinical Evaluation

### 1. Visit Schedule

The visit schedule followed Table 4 below.

TABLE 4
Schedule of Activities

| | Screening | Single Dose | Post-Dose Observation | | | | | | | |
|---|---|---|---|---|---|---|---|---|---|---|
| Time Point | Period | Period | Period | | | Follow-up Period | | | | |
| Item | D 3-D 1 | D 1 | D 2 | D 3 | D 4 | D 7 ± 1 | D 14 ± 1 | D 28 ± 2 | D 56 ± 3 | E/T^{u} ± 3 |
| Sign Informed Consent Form^{a} | X | | | | | | | | | |
| Review Inclusion/Exclusion | X | | | | | | | | | |
| Criteria | | | | | | | | | | |
| Demographic Data | X | | | | | | | | | |
| Medical History | X | | | | | | | | | |
| Vital Signs^{b} | X | X | X | X | X | X | X | X | X | X |
| Physical Examination^{c} | X | | X | | X | X | X | X | X | X |
| Body Height and Weight^{d} | X | | | | | | | | | |
| Hematology^{e} | X | X | X | X | X | X | X | X | X | X |
| Blood Chemistry^{f} | X | X | X | X | X | X | X | X | X | X |
| Coagulation Function^{g} | X | X | X | X | X | X | X | X | X | X |
| Troponin I/Troponin T^{h} | X | X | X | X | X | X | X | X | X | X |
| Urinalysisⁱ | X | X | X | X | X | X | X | X | X | X |
| Pregnancy Test^{j} | X | | | | | | | | | |
| Infectious Disease Markers^{k} | X | | | | | | | | | |
| 12-lead ECG^{l} | X | X | X | X | X | X | X | X | X | X |
| Imaging Examination^{m} | X | | | | | | X | | | |
| CSF Testⁿ | X | X | | X | | X | | | | |
| Electrophysiological | X | | | | | X | X | | | |
| Examination^{v} | | | | | | | | | | |
| Other Hematology Tests^{o} | X | X | | X | | X | | | | |
| Pulse Oximetry Monitoring^{p} | | X | | | | | | | | |
| Infusion Reaction Assessment^{q} | | X | | | | | | | | |
| PK Blood Sampling | | See the collection schedule for pharmacokinetic and anti-drug antibody sample. | | | | | | | | |
| ADA Blood Sample Collection | | | | | | | | | | |
| ASIA and Visual Analog Scale^{r} | X | | X | X | X | X | X | X | X | X |
| AE/SAE^{s} | X | | | | | | | | | |
| Concomitant Medications and | X | | | | | | | | | |
| Treatments^{t} | | | | | | | | | | |

Abbreviations: ADA = Anti-Drug Antibody; AE = Adverse Event; ASIA = American Spinal Cord Injury Association; D = Day; PK = Pharmacokinetics; SAE = Serious Adverse Event; E/T = Early Termination.
^{a}Subjects should sign the ICF prior to any study-related procedures.
^{b}Vital signs examination included body temperature (axillary), respiration, pulse, bp; measurements should be taken at rest. It should be conducted during the screening period; at 5 min ± 1 min, 30 min ± 5 min, 1 h ± 15 min, 1.5 h ± 15 min, 2 h ± 15 min, 4 h ± 30 min, 8 h ± 1 h, and 12 h ± 1 h after dosing initiation on Day 1; on Day 2 (24 h after dosing completion), Day 3, Day 4, Day 7, Day 14, Day 28, Day 56; and upon early termination prior to Day 56.
^{c}Physical Examination: A comprehensive physical examination was required during the screening period, including general condition, skin and mucous membranes of the entire body, superficial lymph nodes, head and neck, chest, abdomen, back, spine and limbs, musculoskeletal system, and nervous system; at other examination time points, a simple physical examination might be conducted (as determined by the investigator), and the investigator might increase the number of examinations at other time points based on the subject's actual condition. The specific time points were during the screening period, Day 2, Day 4, Day 7, Day 14, Day 28, Day 56, and at early withdrawal before Day 56.
^{d}Body Height and Weight: Examination should be performed only at screening (body height could be collected by inquiry, weight must be measured).
^{e}Examination items of Hematology included white blood cell count and differential white blood cell count (lymphocyte count, monocyte count, neutrophil count, eosinophil count, basophil count), red blood cell count, haemoglobin, platelet count, hematocrit, and reticulocyte count; it should be performed at the screening period, 12 h ± 1 h after the end of dosing on D 1, D 2 (24 h after the end of dosing), D 3, D 4, D 7, D 14, D 28, D 56, and upon early withdrawal before D 56. Examination results obtained from the time of spinal cord injury until the signing of the ICF are acceptable during the screening period.
^{f}Examination items of Blood Biochemistry include serum alanine aminotransferase, aspartate aminotransferase, protein total, albumin, globulin, bilirubin total, bilirubin direct, ALP, lactate dehydrogenase, γ-glutamyl transferase, creatinine, glucose, urea/urea nitrogen, uric acid, cholesterol, triglycerides, (electrolytes) potassium, sodium, chloride, calcium, magnesium, phosphorus; it should be conducted during the screening period, 12 h ± 1 h after the end of administration on D 1, D 2 (24 h after the end of administration), D 3, D 4, D 7, D 14, D 28, D 56, and upon early withdrawal before D 56. Examination results obtained from the time of spinal cord injury until the signing of the ICF are acceptable during the screening period.
^{g}Coagulation Function: Prothrombin time, activated partial prothrombin time, international normalized ratio; it should be performed at screening, 12 ± 1 h after the end of dosing on D 1, D 2 (24 h after the end of dosing), D 3, D 4, D 7, D 14, D 28, D 56, and at the time of early withdrawal before D 56. Examination results obtained from the time of spinal cord injury until the signing of the ICF are acceptable during the screening period.
^{h}Troponin I/Troponin T: It should be performed at the screening period, 12 h ± 1 h after the end of dosing on D 1, D 2 (24 h after the end of dosing), D 3, D 4, D 7, D 14, D 28, D 56, and upon early withdrawal before D 56. Examination results obtained from the time of spinal cord injury until the signing of the ICF are acceptable during the screening period.
ⁱExamination items of Urinalysis include pH, specific gravity, protein urine, glucose, white blood cells urine, red blood cells urine, urine ketone body, occult blood, bilirubin, nitrite, urobilinogen. It should be conducted during the screening period, at 12 h ± 1 h after the end of dosing on D 1, on D 2 (24 h after the end of dosing), D 3, D 4, D 7, D 14, D 28, D 56, and at early withdrawal prior to D 56. Examination results obtained from the time of spinal cord injury until the signing of the ICF are acceptable during the screening period.
^{j}Women of childbearing potential should undergo pregnancy test; during the screening period, a serum pregnancy test should be conducted, and for other times, serum/urine pregnancy test should be performed as appropriate.
^{k}Infectious Disease Markers include hepatitis B surface antigen, hepatitis B surface antibody, hepatitis B core antibody, hepatitis C antibody, human immunodeficiency virus antibody, syphilis spirochete antibody (rpr or TRUST). If the hepatitis B surface antigen is positive, further hepatitis B virus DNA testing is required; if hepatitis C antibodies are present, further hepatitis C virus RNA testing is required. Examination results obtained from the time of spinal cord injury until the signing of the ICF are acceptable during the screening period.
^{l}12-lead Electrocardiogram: It should be conducted during the screening period; on Day 1 after the start of dosing at 1 h ± 15 min, 4 h ± 30 min, 8 h ± 1 h, and 12 h ± 1 h; on Day 2 (24 h after the end of dosing); on Days 3, 4, 7, 14, 28, and 56; and upon early withdrawal prior to Day 56. The subject should rest for at least 10 min prior to each examination. If electrocardiogram abnormalities are observed, the frequency of investigation may be increased or a 24-hour electrocardiogram ambulatory may be performed. Examination results obtained from the time of spinal cord injury until the signing of the ICF are acceptable during the screening period.
^{m}Imaging Examinations included MRI, CT, and X-ray modalities, and it should be conducted during the screening period and on D 14. The investigator might increase the number of examinations at other time points based on the subject's actual condition. It was required that the same examination method should be used for the same subject during the study period. Examination results obtained from the time of spinal cord injury until the signing of the ICF are acceptable during the screening period.
ⁿCSF Examination was recommended to be conducted during the screening period, after administration on D 1, and on D 3 and D 7, including detection of ALMB-0166 concentration, S-100, NSE, NG2, GFAP, and other indicators. Cerebrospinal fluid should be collected via lumbar puncture. Investigators might reduce the number of examinations based on the actual circumstances. The examination results would not be used as inclusion or exclusion criteria. Concentration determination of ALMB-0166: Collect at least one sample from each subject, collecting 1 mL of cerebrospinal fluid at each sampling point; S-100, NSE, NG2, GFAP, and other indicator testing: Collect 2 mL of cerebrospinal fluid at each sampling point.
If the investigator could only perform examination once based on actual circumstances, it was recommended to collect 1 mL of cerebrospinal fluid (only for ALMB-0166 concentration determination) immediately after the end of administration up to within 48 h;
If the investigator could perform 2-4 examinations based on actual conditions, one of the following two standards should be followed for collection: (1) If the subject could complete cerebrospinal fluid collection during the screening period, then 3 mL of cerebrospinal fluid should be collected at the screening period and subsequent collection points, and the second collection point should be within 0 to 48 h after the end of administration; (2) If the subject could not undergo cerebrospinal fluid collection during the screening period, then only 1 mL of cerebrospinal fluid needs to be collected at subsequent collection points for the determination of ALMB-0166 concentration, and the first collection point should be within 0 to 48 h after the end of administration.
^{o}Other Hematological Examinations: Including IFN-γ, TNF-α, IL-1β, IL-18, IL-10, IL-6, and TGF-β; it should be conducted during the screening period, 12 h ± 1 h after the end of dosing on D 1, D 3, and D 7, and the results of the examinations are not used as inclusion or exclusion criteria. Examination results obtained from the time of spinal cord injury until the signing of the ICF are acceptable during the screening period.
^{p}Pulse Oxygen Saturation Monitoring: D 1: 15 min ± 3 min before administration and at 30 min ± 5 min, 1 h ± 10 min, 2 h ± 15 min, 4 h ± 30 min, 8 h ± 1 h, and 12 h ± 1 h after the start of administration.
^{q}Assessment of Infusion Reaction: D 1: assessments at 10 min ± 2 min, 1 h ± 10 min, 2 h ± 15 min, 4 h ± 30 min, 8 h ± 1 h, and 12 h ± 1 h after the start of administration.
^{r}ASIA and Visual Analog Scale Scores: ASIA sensory and motor scores, injury grading, and Visual Analog Scale scores. It should be conducted at screening, D 2, D 3, D 4, D 7, D 14, D 28, D 56, and early termination. Examination results obtained from the time of spinal cord injury until the signing of the ICF are acceptable during the screening period.
^{s}Investigators were responsible for collecting and recording all adverse medical events occurring from signing the ICF to within the safety follow-up period (the safety follow-up period is defined as from the first administration to within D 56). After the safety follow-up period, AEs will not be actively collected. If a SAE occurs and the investigator believes there is a reasonable causal relationship with ALMB-0166, it should be reported to the sponsor according to the SAE reporting procedure. AEs (regardless of causality) that are not fully recovered or stabilized at the end of the safety follow-up period must be followed up until recovery (baseline or complete recovery) or clinical stabilization.
^{t}Record all concomitant medications and treatments from signing the ICF to the end of the study.
^{u}Early withdrawal.
^{v}Electrophysiological Examination: Including electromyogram, somatosensory evoked potentials, and motor evoked potential investigation; it should be conducted during the screening period, D 7, and D 14. Qualified sites may conduct electrophysiological examinations. Examination results obtained from the time of spinal cord injury until the signing of the ICF are acceptable during the screening period.

### 2. Efficacy Assessment

After administration, the changes from baseline in ASIA sensory and motor scores, injury classification, and visual analog scale scores should be evaluated at D2, D3, D4, D7, D14, D28, and D56.

### 3. Safety Assessment

a. Vital Signs

Vital signs included body temperature (axillary), respiration, pulse, bp; measurements should be taken at rest.

It should be conducted during the screening period; at 5 min±1 min, 30 min±5 min, 1 h±15 min, 1.5 h±15 min, 2 h±15 min, 4 h±30 min, 8 h±1 h, and 12 h±1 h after dosing initiation on Day 1; on Day 2 (24 h after dosing completion), Day 3, Day 4, Day 7, Day 14, Day 28, Day 56; and upon early termination prior to Day 56.

b. Physical Examination

Physical examinations were determined according to the actual situation. A comprehensive physical examination was required during the screening period, including general condition, skin and mucous membranes of the entire body, superficial lymph nodes, head and neck, chest, abdomen, back, spine and limbs, musculoskeletal system, and nervous system; at other examination time points, a simple physical examination may be conducted (as determined by the investigator).

It should be conducted during the screening period, on D2, D4, D7, D14, D28, D56, and upon early withdrawal before D56.

c. Height and Weight

Body height and weight need to be measured (body height can be collected from the medical interview, weight needs to be measured), and body height and weight information are collected only during screening.

d. Laboratory Tests

If the laboratory measurement results conducted during the screening period according to the inclusion/exclusion criteria exceeded the ranges specified by the protocol, the test could be repeated once as soon as possible before enrollment to rule out laboratory errors. If the repeated measurement still exceeded the protocol-specified range, the subject should be excluded from the study.

If the range of a certain laboratory measurement was not specified in the protocol, but the measurement during the screening period exceeded the normal range of the study site, whether it had clinical significance should be determined by the investigator based on the nature and extent of the observed abnormality. This test could be repeated as soon as possible and performed prior to enrollment to rule out laboratory error.

In all cases, the investigator must document the clinical decision as to whether the subject was allowed to continue the study (i.e., whether the result was clinically significant and/or medically relevant) in the source documents.

Clinically significant and/or ALMB-0166-related test results should be recorded on the comments page of the eCRF, and the date, study day, and specific time should be noted.
i. Hematology: including included white blood cell count, differential white blood cell count (lymphocyte count, monocyte count, neutrophil count, eosinophil count, basophil count), red blood cell count, haemoglobin, platelet count, hematocrit, and reticulocyte count. It should be conducted during the screening period, at 12 h±1 h after the end of dosing on D1, on D2 (24 h after the end of dosing), D3, D4, D7, D14, D28, D56, and at early withdrawal prior to D56. Examination results obtained from the time of spinal cord injury until the signing of the ICF are acceptable during the screening period.
ii. Blood Chemistry: examination items of Blood Biochemistry included serum alanine aminotransferase, aspartate aminotransferase, protein total, albumin, globulin, bilirubin total, bilirubin direct, ALP, lactate dehydrogenase, γ-glutamyl transferase, creatinine, glucose, urea/urea nitrogen, uric acid, cholesterol, triglycerides, (electrolytes) potassium, sodium, chloride, calcium, magnesium, phosphorus. It should be conducted during the screening period, at 12 h±1 h after the end of dosing on D1, on D2 (24 h after the end of dosing), D3, D4, D7, D14, D28, D56, and at early withdrawal prior to D56. Examination results obtained from the time of spinal cord injury until the signing of the ICF are acceptable during the screening period
iii. Coagulation Function: it includes prothrombin time, partial prothrombin time, international normalized ratio. It should be conducted during the screening period, at 12 h±1 h after the end of dosing on D1, on D2 (24 h after the end of dosing), D3, D4, D7, D14, D28, D56, and at early withdrawal prior to D56. Examination results obtained from the time of spinal cord injury until the signing of the ICF are acceptable.
iv. Urinalysis: Examination items of Urinalysis include pH, specific gravity, protein urine, glucose, white blood cells urine, red blood cells urine, urine ketone body, occult blood, bilirubin, nitrite, urobilinogen. It should be conducted during the screening period, at 12 h±1 h after the end of dosing on D1, on D2 (24 h after the end of dosing), D3, D4, D7, D14, D28, D56, and at early withdrawal prior to D56. Examination results obtained from the time of spinal cord injury until the signing of the ICF are acceptable during the screening period.
v. Troponin I/Troponin T: It should be conducted during the screening period, at 12 h±1 h after the end of dosing on D1, on D2 (24 h after the end of dosing), D3, D4, D7, D14, D28, D56, and at early withdrawal prior to D56. Examination results obtained from the time of spinal cord injury until the signing of the ICF are acceptable during the screening period.
vi. Virological Screening: virology screening should be conducted during the screening period, but the results were not used as inclusion criteria period. It includes hepatitis B surface antigen, hepatitis B surface antibody, hepatitis B core antibody, hepatitis C antibody, human immunodeficiency virus antibody, syphilis spirochete antibody (rpr or TRUST). If the hepatitis B surface antigen is positive, further hepatitis B virus DNA testing is required; if hepatitis C antibodies are present, further hepatitis C virus RNA testing is required. Examination results obtained from the time of spinal cord injury until the signing of the ICF are acceptable during the screening period.
vii. Serum/Urine Pregnancy: Women of childbearing potential needed to undergo pregnancy detection; during the screening period, a serum pregnancy test was conducted, and other times, serum/urine pregnancy test was performed as appropriate.
   e. lead ECGs

It should be conducted during the screening period; on Day 1 after the start of dosing at 1 h±15 min, 4 h±30 min, 8 h±1 h, and 12 h±1 h; on Day 2 (24 h after the end of dosing); on Days 3, 4, 7, 14, 28, and 56; and upon early withdrawal prior to Day 56. Examination results obtained from the time of spinal cord injury until the signing of the ICF are acceptable during the screening period. The ECG report should include an overall assessment and confirm whether there are clinically significant abnormal findings and, if so, it should be described in detail. The signed original ECG report will be archived at the study site.

f. Pulse Oxygen Saturation Monitoring

Pulse oxygen saturation will be measured at 15 min±3 min before dosing, and at 30 min±5 min, 1 min±10 min, 2 h±15 min, 4 h±30 min, 8 h±1 h, and 12 h±1 h after the start of dosing. Pulse oxygen saturation values will be recorded in the eCRF.

g. Imaging Examination

Imaging examinations using MRI, CT, X-ray, and other examination methods were conducted during the screening period and on D14. Investigators may increase the number of investigations at other time points according to the actual condition of the subjects. It was required that the same examination methods should be used for the same subject during the study. Examination results obtained from the time of spinal cord injury until the signing of the JCF are acceptable during the screening period.

### 4. PK and Immunogenicity Assessment

Blood samples were collected for PK assessment. For blood PK, collection time points are shown in Table 5, approximately 3.5 mL of venous whole blood was collected at each collection point. Sample collection times are detailed in Table 5; immunogenicity of ALMB-0166 was assessed by the incidence of ADA.

TABLE 5
Sampling Schedule of PK and ADA Samples
for Single-Dose Dose Escalation Study.

| Study | | | | | |
|---|---|---|---|---|---|
| Duration | Sampling Time Point | Time Window | | PK | ADA |
| D 1 | Pre-dose | −30 | min | ✓ | ✓ |
| | Immediately after the | +5 | min | ✓ | |
| | end of dosing | | | | |
| | 2 h after the start of | ±5 | min | ✓ | |
| | dosing | | | | |
| | 5 h after the start of | ±10 | min | ✓ | |
| | dosing | | | | |
| | 9 h after the start of | ±10 | min | ✓ | |
| | dosing | | | | |
| D 2 | 24 h after the start of | ±1 | hour | ✓ | |
| | dosing | | | | |
| D 3 | 48 h after the start of | ±2 | hour | ✓ | |
| | dosing | | | | |
| D 4 | 72 h after the start of | ±2 | hour | ✓ | |
| | dosing | | | | |

| D 7 | Within the corresponding | ✓ | |
|---|---|---|---|
| D 14 | visit window | ✓ | ✓ |
| D 28 | | ✓ | ✓ |
| D 56 | | ✓ | ✓ |
| Early | | ✓ | ✓ |
| Withdrawal | | | |

### E. Determination of Statistical Methods and Sample Size

### 1. Analysis Datasets

FAS: Defined as all subjects who were successfully randomized and received at least one dose of ALMB-0166 according to the intention-to-treat principle. FAS will be used for the analysis of demographics and baseline characteristics.
PKCS: Including all subjects who used ALMB-0166 and had at least one measurable concentration. Reasons for excluding certain drug concentrations of a subject from the PK analysis set include but are not limited to the following: factors affecting drug concentrations (e.g., incorrect treatment); improper handling of samples; incorrect testing, etc.
PKPS: Including all subjects who received ALMB-0166 and had at least one evaluable PK parameter. Subjects who were excluded from PKPS included but were not limited to: subjects with major protocol deviations (inclusion/exclusion criteria) that affected the PK parameter results, or rendered the parameters not estimable; subjects who used concomitant medications during the study, which affected the PK parameters.
SS: Including all subjects who received at least one dose of ALMB-0166.
IS: Including all enrolled subjects who have received at least one dose of ALMB-0166, and have baseline and at least one post-baseline immunogenicity assessment data.
PDS: Including all enrolled subjects who have received at least one dose of ALMB-0166, and have baseline and at least one post-baseline pharmacodynamic evaluation data.

### 2. General Principles of Statistical Analysis

Unless otherwise specified, “baseline” in this study is defined as the last non-missing measurement (including unscheduled visits) prior to the first dose of ALMB-0166, including measurements taken on the day of the first dose and prior to the first dose.

In general, only critical missing dates were imputed, unless otherwise specified.

Unless otherwise specified, quantitative variables were statistically described using the number, mean, standard deviation (SD), median, minimum, and maximum. Variables that may not follow a normal distribution can be described using the number, upper quartile, lower quartile, median, maximum, and minimum. Geometric means and coefficients of variation of the geometric means would also be provided if needed.

For qualitative indicators, the frequency and percentage of the corresponding composition were calculated.

### 3. Subject Disposition

Subject disposition were summarized descriptively, including the entire process from subject screening to study completion.

The number and percentage of subjects with successful screening, failed screening, and reasons for screening failures were calculated based on all screened subjects.

Based on the randomized population, the number and percentage of subjects for enrollment, dosing, reasons for drug administration interrupted, early withdrawal from the study, and reasons for early withdrawal from the study were summarized.

Based on the randomized population, the number of subjects included in each analysis set was summarized.

All subjects who received randomization were tabulated, including subject ID, dosing date and time, reasons for drug administration interrupted, study end date, and reasons for study termination, etc.

The listing showed detailed information on subjects included, dropped out, or excluded from each analysis set.

### 4. Protocol Deviations

Protocol deviations were identified, documented, and categorized according to the list of protocol deviation definition during the study. Prior to database lock, the main study team members reviewed all protocol deviations. Deviations that seriously affect the rights, safety, and benefits of the subjects, or the integrity, accuracy, and reliability of the study data were judged as major protocol deviations. Major protocol deviations were described in detail in the protocol deviation description document, and the final decision on whether to exclude them from each analysis set was made before database lock.

The number and percentage of subjects with different types of major protocol deviations were summarized by dose group based on the randomized population. All major protocol deviations were tabulated.

### 5. Demographic Data and Baseline Characteristics

a. Demographics and Baseline Characteristics

Descriptive statistical analyses of demographics and other baseline characteristics were performed by dose group based on the full analysis set. The number and percentage of each category were calculated for qualitative data, and quantitative data was summarized by descriptive statistics (mean, SD, median, minimum and maximum, etc.).

Detailed listings of demographics and other baseline data were provided.

Summarized demographic and baseline characteristics included: age, sex, ethnicity, body height, weight, and body mass index.

Summarized disease baseline characteristics included: spinal cord injury sites and infectious disease markers.

b. Baseline Disease Characteristics

Based on FAS, the following disease baseline characteristics were summarized: spinal cord injury sites, infectious disease markers.

c. Baseline Disease Characteristics

MedDRA version 27.0 was used to code the general medical history recorded in the eCRF.

Based on the FAS, the number and percentage of subjects were summarized with all relevant medical histories according to SOC and PT. All recorded relevant medical histories were tabulated.

d. Prior Medical History

MedDRA version 27.0 was used to code the general medical history recorded in the eCRF.

Based on the FAS, the number and percentage of subjects were summarized with all relevant medical histories according to SOC and PT.

All recorded relevant medical histories were tabulated.

e. Prior and Concomitant Medications and Non-Drug Therapies

Prior and concomitant medications were coded using WHO Drug GLOBAL B3 Mar24 and classified according to the appropriate ATC, and summarize by ATC2 and PN. Prior and concomitant non-drug therapies were coded using MedDRA version 27.0, and summarized according to SOC and PT.

### 6. PK Analysis Methods

a. Analysis of Plasma Concentrations

Based on PKCS, according to the scheduled sampling times, listing and descriptive statistical analysis of ALMB-0166 plasma concentration data were performed by dose group; descriptive statistics included: N, Nₘᵢₛₛ, number of BQL (n of BQL), Mean, SD, % CV, Median, Min, Max, Geo.Mean, Geo.SD, % CV_{b}. The mean plasma concentration-time curves of subjects were plotted by dose group (linear and logarithmic coordinates); according to actual sampling times, individual overlaid plasma concentration-time curves were plotted by dose group (linear and logarithmic coordinates).

b. PK Parameter Analysis

Based on PKPS, the PK parameters were tabulated and descriptive statistical analyses were performed for each dose group. The statistical metrics included: except for Tₘₐₓ, calculate Mean, SD, % CV, Median, Min, Max, Q1, Q3, Geo.Mean, Geo.SD, and % CV_{b} for all other parameters. For Tₘₐₓ, only Median, Min, Max, Q1, and Q3 were calculated. For both subjects whose sampling time points were within the time window and those whose sampling time points were outside the time window, PK parameters were calculated based on the actual sampling time.

If sufficient data is available, the data of all the above-mentioned subjects will be analyzed using a nonlinear mixed-effects model for PopPK analysis; please refer to a separate report for details and results of the analysis.

### 7. Immunogenicity Analysis

Based on the IS, the number and percentage of subjects with positive ADA samples were summarized by dose group and visit. The number and percentage of subjects with ADA-positive baseline samples were summarized by dose group; the number and proportion of subjects who were positive at baseline and whose ADA titers in positive post-treatment samples did not increase were summarized; in the condition of data permission, descriptive statistical analysis was performed on the titers of baseline positive samples.

The number and percentage of subjects who were ADA negative at baseline and ADA positive post-baseline were summarized by dose group; the number and percentage of subjects who were ADA positive at baseline and had post-baseline titers ≥4 times that of the baseline ADA positive samples were summarized. Descriptive statistical analysis was performed on the increase in titer, if data permit. If data permit, the mean titer ratio-time curve and the median titer ratio-time curve would be plotted according to the planned sampling time points.

If applicable, exploratory analyses would be performed to evaluate the impact of immunogenicity on PK, safety, and efficacy.

The listings of immunogenicity-related data results were tabulated.

### II. Study Population

### A. Subject Disposition

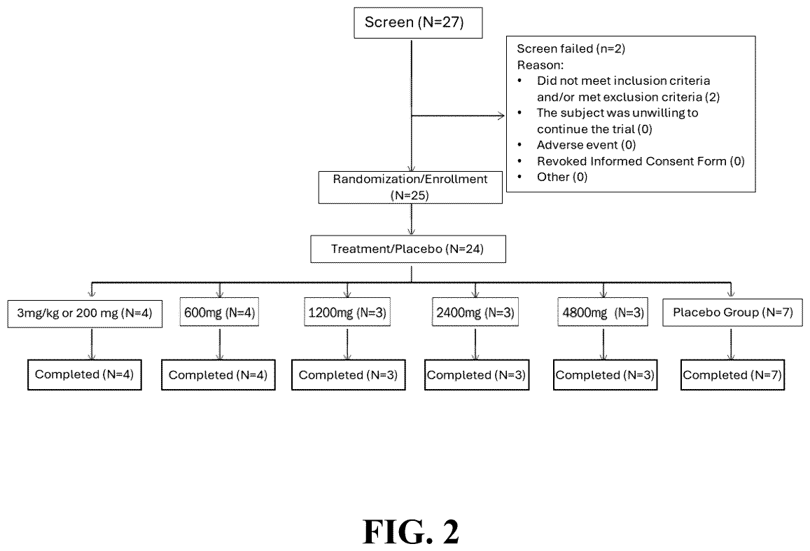

The subject distribution diagram is shown in FIG. 2.

A total of 27 subjects signed the ICF and participated in screening, among whom 2 subjects (7.4%) failed the screening, all due to not meeting the inclusion criteria and/or meeting the exclusion criteria.

After successful screening, 25 subjects were enrolled. One subject (4.0%) withdrew early due to unwillingness to continue participating in the study, and 24 subjects (96.0%) received treatment. Among them, 4 subjects were in each of the 200 mg (or 3 mg/kg) and 600 mg groups; 3 subjects were in each of the 1200 mg, 2400 mg, and 4800 mg groups; and 7 subjects were in the placebo group.

All 24 subjects who received the study treatment completed the study.

### B. Protocol Deviations

Among the randomized population (25 subjects), a total of 2 subjects (8.0%) experienced at least one major protocol deviation, all of whom were in the placebo group and had used prohibited drug therapy. A summary of major protocol deviations during the study in the randomized population is presented in Table 6.

TABLE 6
Major Protocol Deviations (Randomized Population)

| | 3 mg/kg | | | | | | |
|---|---|---|---|---|---|---|---|
| | or 200 | | | | | Placebo | |
| | mg | 600 mg | 1200 mg | 2400 mg | 4800 mg | Group | Total |
| | (N = 4) | (N = 4) | (N = 3) | (N = 3) | (N = 3) | (N = 8) | (N = 25) |
| | n (%) | n (%) | n (%) | n (%) | n (%) | n (%) | n (%) |
| At Least One | 0 | 0 | 0 | 0 | 0 | 2 (25.0) | 2 (8.0) |
| Major Protocol | | | | | | | |
| Deviation | | | | | | | |
| Occurred | | | | | | | |
| Prohibited | 0 | 0 | 0 | 0 | 0 | 2 (25.0) | 2 (8.0) |
| Medications/ | | | | | | | |
| Treatments | | | | | | | |

### III. Efficacy/PK/PD Results

### A. Analysis Datasets

A total of 25 subjects were enrolled in this study, among whom 24 received at least one study treatment.

Twenty-four subjects were included in the FAS and SS datasets, 17 subjects were included in the PKCS and PKPS datasets, 21 subjects were included in the IS dataset, and 16 subjects were included in the PDS dataset.

A summary of each analysis dataset is provided in Table 7.

TABLE 7
Analysis Datasets (Randomized Population).

| | 3 mg/kg | | | | | | |
|---|---|---|---|---|---|---|---|
| | or 200 | | | | | Placebo | |
| | mg | 600 mg | 1200 mg | 2400 mg | 4800 mg | Group | Total |
| | (N = 4) | (N = 4) | (N = 3) | (N = 3) | (N = 3) | (N = 8) | (N = 25) |
| | n (%) | n (%) | n (%) | n (%) | n (%) | n (%) | n (%) |
| FAS | 4 (100) | 4 (100) | 3 (100) | 3 (100) | 3 (100) | 7 (87.5) | 24 (96.0) |
| PKCS | 4 (100) | 4 (100) | 3 (100) | 3 (100) | 3 (100) | 0 | 17 (68.0) |
| PKPS | 4 (100) | 4 (100) | 3 (100) | 3 (100) | 3 (100) | 0 | 17 (68.0) |
| SS | 4 (100) | 4 (100) | 3 (100) | 3 (100) | 3 (100) | 7 (87.5) | 24 (96.0) |
| IS | 4 (100) | 3 (75.0) | 3 (100) | 2 (66.7) | 3 (100) | 6 (75.0) | 21 (84.0) |
| PDS | 4 (100) | 4 (100) | 3 (100) | 0 | 0 | 5 (62.5) | 16 (64.0) |

Abbreviations: FAS = Full Analysis Set; IS = Immunization Analysis Set; PKCS = Pharmacokinetic Concentration Set; PKPS = Pharmacokinetic Parameter Set; PDS = Pharmacodynamic Analysis Set; SS = Safety Analysis Set.

### B. Demographic Data and Baseline Characteristics

### 1. Demographics

Demographic information and baseline characteristics are shown in Table 8.

Based on the FAS, the median age of the subjects was 57.5 years (range: 39-74), 70.8% were male, 95.8% were of Han, and the mean weight±standard deviation was 72.2±13.59 kg. Baseline demographic characteristics were generally similar across all groups.

The median ages were similar across groups (200 mg [or 3 mg/kg], 600 mg, 1200 mg, 2400 mg, 4800 mg, and placebo groups), being 52.0, 61.0, 56.0, 61.0, 55.0, and 57.0 years, respectively.

The gender proportions were similar across groups, with males accounting for 75.0%, 75.0%, 66.7%, 66.7%, 66.7%, and 71.4%, respectively.

The mean weights of each group were 75.3 kg, 84.0 kg, 58.3 kg, 78.3 kg, 63.3 kg, and 70.7 kg. The mean weight of 1200 mg dose group was slightly lower.

TABLE 8
Demographic Information and Baseline Characteristics (FAS).

| | 3 mg/kg | | | | | Placebo | |
|---|---|---|---|---|---|---|---|
| | or 200 mg | 600 mg | 1200 mg | 2400 mg | 4800 mg | Group | Total |
| | (N = 4) | (N = 4) | (N = 3) | (N = 3) | (N = 3) | (N = 7) | (N = 24) |
| Age (years) | | | | | | | |
| Number of | 4 | 4 | 3 | 3 | 3 | 7 | 24 |
| Subjects | | | | | | | |
| Mean (Standard | 55.0 (11.20) | 58.0 (13.54) | 54.3 (12.58) | 65.0 (7.81) | 55.7 (10.02) | 58.7 (6.26) | 57.8 (9.43) |
| Deviation) | | | | | | | |
| Median | 52.0 | 61.0 | 56.0 | 61.0 | 55.0 | 57.0 | 57.5 |
| Quartiles | 48.0, 62.0 | 49.5, 66.5 | 41.0, 66.0 | 60.0, 74.0 | 46.0, 66.0 | 54.0, 67.0 | 53.0, 66.0 |
| (Q1, Q3) | | | | | | | |
| Minimum, | 45, 71 | 39, 71 | 41, 66 | 60, 74 | 46, 66 | 53, 68 | 39, 74 |
| Maximum | | | | | | | |
| Sex, n (%) | | | | | | | |
| Male | 3 (75.0) | 3 (75.0) | 2 (66.7) | 2 (66.7) | 2 (66.7) | 5 (71.4) | 17 (70.8) |
| Female | 1 (25.0) | 1 (25.0) | 1 (33.3) | 1 (33.3) | 1 (33.3) | 2 (28.6) | 7 (29.2) |
| Ethnicity, n (%) | | | | | | | |
| Han | 4 (100) | 3 (75.0) | 3 (100) | 3 (100) | 3 (100) | 7 (100) | 23 (95.8) |
| Other ethnicities | 0 | 1 (25.0) | 0 | 0 | 0 | 0 | 1 (4.2) |
| Height (cm) | | | | | | | |
| Number of | 4 | 4 | 3 | 3 | 3 | 7 | 24 |
| Subjects | | | | | | | |
| Mean (Standard | 168.0 (8.12) | 174.8 (10.24) | 163.3 (3.06) | 166.0 (16.37) | 168.0 (8.89) | 169.0 (7.85) | 168.6 (8.98) |
| Deviation) | | | | | | | |
| Median | 168.5 | 178.0 | 164.0 | 170.0 | 171.0 | 170.0 | 170.0 |
| Quartiles | 161.0, 175.0 | 168.0, 181.5 | 160.0, 166.0 | 148.0, 180.0 | 158.0, 175.0 | 160.0, 175.0 | 160.0, 175.0 |
| (Q1, Q3) | | | | | | | |
| Minimum, | 160, 175 | 160, 183 | 160, 166 | 148, 180 | 158, 175 | 158, 180 | 148, 183 |
| Maximum | | | | | | | |
| Weight (kg) | | | | | | | |
| Number of | 4 | 4 | 3 | 3 | 3 | 7 | 24 |
| Subjects | | | | | | | |
| Mean (Standard | 75.3 (9.11) | 84.0 (20.61) | 58.3 (8.14) | 78.3 (16.07) | 63.3 (5.77) | 70.7 (8.90) | 72.2 (13.59) |
| Deviation) | | | | | | | |
| Median | 74.5 | 86.5 | 62.0 | 85.0 | 60.0 | 68.0 | 68.0 |
| Quartiles | 67.5, 83.0 | 68.0, 100.0 | 49.0, 64.0 | 60.0, 90.0 | 60.0, 70.0 | 64.0, 78.0 | 61.0, 83.0 |
| (Q1, Q3) | | | | | | | |
| Minimum, | 67, 85 | 58, 105 | 49, 64 | 60, 90 | 60, 70 | 60, 85 | 49, 105 |
| Maximum | | | | | | | |

### 2. Baseline Disease Characteristics

Baseline disease characteristics are shown in Table 9.

Based on FAS, the number and percentage of subjects whose spinal column injury involved C3, C4, C5, C6, and C7 were respectively 6 subjects (25.0%), 16 subjects (66.7%), 18 subjects (75.0%), 14 subjects (58.3%), and 4 subjects (16.7%).

The numbers and proportions of subjects with C3 injury in each group (200 mg [or 3 mg/kg], 600 mg, 1200 mg, 2400 mg, 4800 mg, and placebo groups) were 1 subject (25.0%), 2 subjects (50.0%), 1 subject (33.3%), 0, 0, and 2 subjects (28.6%), respectively. The numbers and proportions of subjects with C4 injury in each group were 3 subjects (75.0%), 3 subjects (75.0%), 1 subject (33.3%), 3 subjects (100%), 1 subject (33.3%), and 5 subjects (71.4%), respectively. The numbers and proportions of subjects with C5 injury in each group were 4 subjects (100%), 3 subjects (75.0%), 2 subjects (66.7%), 3 subjects (100%), 3 subjects (100%), and 3 subjects (42.9%), respectively. The numbers and proportions of subjects with C6 injury in each group were 2 subjects (50.0%), 4 subjects (100%), 1 subject (33.3%), 3 subjects (100%), 2 subjects (66.7%), and 2 subjects (28.6%), respectively. The numbers of subjects with C7 injury in each group were 1 subject (25.0%), 2 subjects (50.0%), 0, 1 subject (33.3%), 0, and 0, respectively.

The results of virological tests of subjects in each group showed normal or clinically insignificant abnormalities: hepatitis B surface antigen, hepatitis C antibody, hepatitis B virus DNA, hepatitis C virus RNA, HIV antibody, and syphilis spirochete antibody were all normal; hepatitis B surface antibody and hepatitis B core antibody were all normal or clinically insignificant abnormalities.

TABLE 9
Baseline Disease Characteristics (FAS).

| | 3 mg/kg or | | | | | Placebo | |
|---|---|---|---|---|---|---|---|
| | 200 mg | 600 mg | 1200 mg | 2400 mg | 4800 mg | Group | Total |
| | (N = 4) | (N = 4) | (N = 3) | (N = 3) | (N = 3) | (N = 7) | (N = 24) |
| | n (%) | n (%) | n (%) | n (%) | n (%) | n (%) | n (%) |
| Spinal Column Injury | | | | | | | |
| Location (Multiple | | | | | | | |
| Selections) | | | | | | | |
| C3 | 1 (25.0) | 2 (50.0) | 1 (33.3) | 0 | 0 | 2 (28.6) | 6 (25.0) |
| C4 | 3 (75.0) | 3 (75.0) | 1 (33.3) | 3 (100) | 1 (33.3) | 5 (71.4) | 16 (66.7) |
| C5 | 4 (100) | 3 (75.0) | 2 (66.7) | 3 (100) | 3 (100) | 3 (42.9) | 18 (75.0) |
| C6 | 2 (50.0) | 4 (100) | 1 (33.3) | 3 (100) | 2 (66.7) | 2 (28.6) | 14 (58.3) |
| C7 | 1 (25.0) | 2 (50.0) | 0 | 1 (33.3) | 0 | 0 | 4 (16.7) |
| Hepatitis B Surface | | | | | | | |
| Antigen | | | | | | | |
| Normal | 3 (75.0) | 2 (50.0) | 3 (100) | 3 (100) | 3 (100) | 6 (85.7) | 20 (83.3) |
| Clinically Insignificant | 0 | 0 | 0 | 0 | 0 | 0 | 0 |
| Abnormality | | | | | | | |
| Clinically Significant | 0 | 0 | 0 | 0 | 0 | 0 | 0 |
| Abnormality | | | | | | | |
| Hepatitis B Surface | | | | | | | |
| Antibody | | | | | | | |
| Normal | 2 (50.0) | 1 (25.0) | 2 (66.7) | 0 | 2 (66.7) | 5 (71.4) | 12 (50.0) |
| Clinically Insignificant | 1 (25.0) | 1 (25.0) | 1 (33.3) | 3 (100) | 1 (33.3) | 1 (14.3) | 8 (33.3) |
| Abnormality | | | | | | | |
| Clinically Significant | 0 | 0 | 0 | 0 | 0 | 0 | 0 |
| Abnormality | | | | | | | |
| Hepatitis B Core Antibody | | | | | | | |
| Normal | 1 (25.0) | 1 (25.0) | 1 (33.3) | 1 (33.3) | 2 (66.7) | 5 (71.4) | 11 (45.8) |
| Clinically Insignificant | 2 (50.0) | 1 (25.0) | 2 (66.7) | 2 (66.7) | 1 (33.3) | 1 (14.3) | 9 (37.5) |
| Abnormality | | | | | | | |
| Clinically Significant | 0 | 0 | 0 | 0 | 0 | 0 | 0 |
| Abnormality | | | | | | | |
| Hepatitis C antibody | | | | | | | |
| Normal | 3 (75.0) | 2 (50.0) | 3 (100) | 2 (66.7) | 3 (100) | 6 (85.7) | 19 (79.2) |
| Clinically Insignificant | 0 | 0 | 0 | 0 | 0 | 0 | 0 |
| Abnormality | | | | | | | |
| Clinically Significant | 0 | 0 | 0 | 0 | 0 | 0 | 0 |
| Abnormality | | | | | | | |
| Hepatitis B virus DNA | | | | | | | |
| Normal | 1 (25.0) | 0 | 0 | 0 | 0 | 2 (28.6) | 3 (12.5) |
| Clinically Insignificant | 0 | 0 | 0 | 0 | 0 | 0 | 0 |
| Abnormality | | | | | | | |
| Clinically Significant | 0 | 0 | 0 | 0 | 0 | 0 | 0 |
| Abnormality | | | | | | | |
| Hepatitis C virus RNA | | | | | | | |
| Normal | 1 (25.0) | 1 (25.0) | 0 | 0 | 0 | 2 (28.6) | 4 (16.7) |
| Clinically Insignificant | 0 | 0 | 0 | 0 | 0 | 0 | 0 |
| Abnormality | | | | | | | |
| Clinically Significant | 0 | 0 | 0 | 0 | 0 | 0 | 0 |
| Abnormality | | | | | | | |
| Human Immunodeficiency | | | | | | | |
| Virus Antibody | | | | | | | |
| Normal | 3 (75.0) | 2 (50.0) | 3 (100) | 2 (66.7) | 3 (100) | 6 (85.7) | 19 (79.2) |
| Clinically Insignificant | 0 | 0 | 0 | 0 | 0 | 0 | 0 |
| Abnormality | | | | | | | |
| Clinically Significant | 0 | 0 | 0 | 0 | 0 | 0 | 0 |
| Abnormality | | | | | | | |
| Treponema Pallidum | | | | | | | |
| Antibody | | | | | | | |
| Normal | 3 (75.0) | 2 (50.0) | 3 (100) | 2 (66.7) | 3 (100) | 6 (85.7) | 19 (79.2) |
| Clinically Insignificant | 0 | 0 | 0 | 0 | 0 | 0 | 0 |
| Abnormality | | | | | | | |
| Clinically Significant | 0 | 0 | 0 | 0 | 0 | 0 | 0 |
| Abnormality | | | | | | | |

Abbreviations: C = cervical; DNA = deoxyribonucleic acid; RNA = ribonucleic acid.
Note:
Percentages are based on the number of FAS subjects in each group.

### 3. Prior and Concomitant Medical History

Based on FAS, all 24 subjects (100%) had prior and concomitant medical histories. According to PN statistics, medical histories with an incidence of ≥30% included cervical spinal cord injury in 17 subjects (70.8%), intervertebral disc herniation in 16 subjects (66.7%), spinal osteoarthritis and hypertension in 11 subjects each (45.8%). 4. Prior and Concomitant Medications and Therapies

a. Prior Medications and Therapies

Based on FAS, 20 subjects (83.3%) had a history of prior drug therapy. According to SOC statistics, prior drug therapies with an incidence of ≥30% included systemic corticosteroids, blood substitutes, and perfusion solutions in 12 subjects each (50.0%), and medications used for acid-related diseases in 11 subjects (45.8%). The proportions of previous drug therapy were similar across groups (200 mg [or 3 mg/kg] group, 600 mg group, 1200 mg group, 2400 mg group, 4800 mg group, and placebo group), being 50.0%, 100%, 100%, 100%, 100%, and 71.4% respectively.

Four subjects (16.7%) had a history of prior non-drug therapy, including one case (4.2%) each of open reduction of fracture, rehabilitation therapy, debridement, and hernia repair.

b. Concomitant Medications and Therapies

Based on the FAS, all 24 subjects (100%) had concomitant medications. According to SOC statistics, concomitant medications with an incidence ≥50% included 22 subjects (91.7%) each of systemic antibacterial agents, systemic corticosteroids, blood substitutes and perfusion solutions; 19 subjects (79.2%) of drugs for acid-related diseases; 18 subjects (75.0%) each of anti-inflammatory and anti-rheumatic drugs, and drugs used for constipation; 17 subjects (70.8%) of other nervous system drugs; 16 subjects (66.7%) each of antithrombotic drugs and analgesics; 15 subjects (62.5%) of cough and cold medications; 13 subjects (54.2%) of anesthetics; and 12 subjects (50.0%) of psycholeptics.

Seven subjects (29.2%) had concomitant non-drug therapies, including 5 subjects (20.8%) of pneumatic therapy, 4 subjects (16.7%) of rehabilitation therapy, 3 subjects (12.5%) of microwave therapy, 2 subjects (8.3%) of acupuncture, and 1 subject (4.2%) each of mechanical ventilation, cooling therapy, and vena cava filter insertion.

C. Analysis of Compliance with Study Intervention

Medication compliance is shown in Table 10.

Based on the SS, the median medication compliance in each group was 100% (range: 100, 100).

TABLE 10
Medication Compliance (SS)

| | 3 mg/kg or | | | | | Placebo | |
|---|---|---|---|---|---|---|---|
| | 200 mg | 600 mg | 1200 mg | 2400 mg | 4800 mg | Group | Total |
| | (N = 4) | (N = 4) | (N = 3) | (N = 3) | (N = 3) | (N = 7) | (N = 24) |
| Medication | | | | | | | |
| Compliance (%) | | | | | | | |
| Number of | 4 | 4 | 3 | 3 | 3 | 7 | 24 |
| Subjects | | | | | | | |
| (Missing) | | | | | | | |
| Mean (Standard | 100.0 (0.00) | 100.0 (0.00) | 100.0 (0.00) | 100.0 (0.00) | 100.0 (0.00) | 100.0 (0.00) | 100.0 (0.00) |
| Deviation) | | | | | | | |
| Median | 100.0 | 100.0 | 100.0 | 100.0 | 100.0 | 100.0 | 100.0 |
| Quartiles (Q1, Q3) | 100.0, 100.0 | 100.0, 100.0 | 100.0, 100.0 | 100.0, 100.0 | 100.0, 100.0 | 100.0, 100.0 | 100.0, 100.0 |
| Minimum, | 100.0, 100.0 | 100.0, 100.0 | 100.0, 100.0 | 100.0, 100.0 | 100.0, 100.0 | 100.0, 100.0 | 100.0, 100.0 |
| Maximum | | | | | | | |
| Classification of | | | | | | | |
| Medication | | | | | | | |
| Compliance, n (%) | | | | | | | |
| <80% | 0 | 0 | 0 | 0 | 0 | 0 | 0 |
| 80%-120% | 4 (100) | 4 (100) | 3 (100) | 3 (100) | 3 (100) | 7 (100) | 24 (100) |
| >120% | 0 | 0 | 0 | 0 | 0 | 0 | 0 |

Note:
Medication Compliance: Actual dose/Planned dose*100%.

### D. Efficacy/PK/PD Data Results and Individual Subject Data Listings

### 1. Efficacy Analysis

a. change from Baseline in ASIA Sensory Score
i. Total ASIA Sensory Score

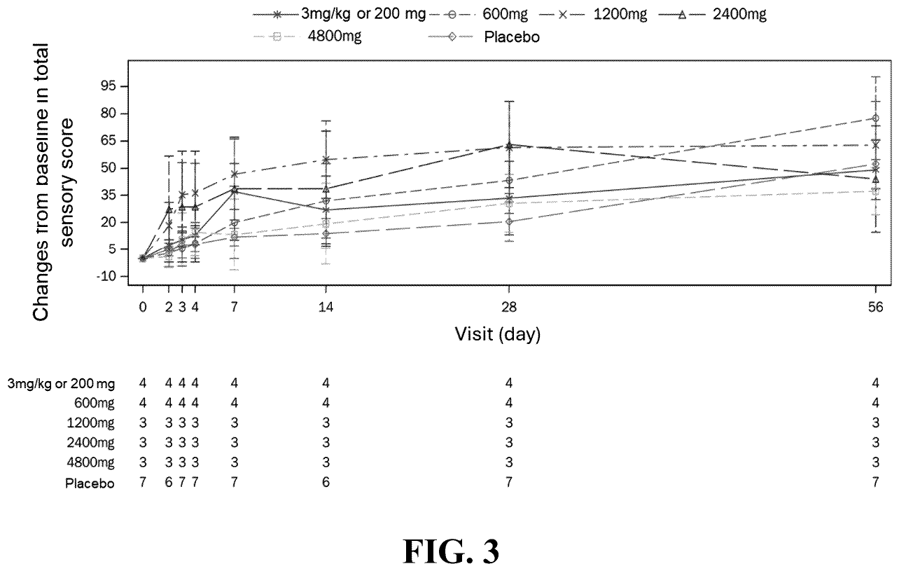

Based on the FAS, the time curve of the mean±SE change from baseline in total ASIA sensory score is shown in FIG. 3. The analysis of the change from baseline in total ASIA sensory score is presented in Table 11.

After receiving the study intervention, the mean change from baseline (standard deviation) in total ASIA sensory score evaluated on D2, D3, D4, D7, D14, D28, and D56 for subjects in the 200 mg (or 3 mg/kg) group were 7.5 (5.97), 10.5 (7.72), 13.0 (10.65), 37.0 (31.09), 27.0 (37.26), 33.5 (40.54), and 49.0 (32.60), respectively.

After receiving the study intervention, the mean change from baseline (standard deviation) in total ASIA sensory score evaluated on D2, D3, D4, D7, D14, D28, and D56 for subjects in the 600 mg group were 3.0 points (15.10), 5.5 points (19.82), 8.5 points (17.00), 20.0 points (40.13), 32.0 points (40.92), 43.3 points (36.25), and 77.5 points (45.59), respectively.

After receiving the study intervention, the mean change from baseline (standard deviation) in total ASIA sensory score evaluated on D2, D3, D4, D7, D14, D28, and D56 for subjects in the 1200 mg group were 18.3 points (22.19), 35.3 points (30.75), 36.3 points (28.22), 46.7 points (33.55), 54.7 points (36.95), 61.3 points (43.88), and 62.7 points (41.63), respectively.

After receiving the study intervention, the mean change from baseline (standard deviation) in total ASIA sensory score evaluated on D2, D3, D4, D7, D14, D28, and D56 for subjects in the 2400 mg group were 27.3 points (50.85), 28.7 points (53.15), 28.7 points (53.15), 38.7 points (49.17), 38.7 points (54.93), 63.0 points (41.15), and 44.0 points (50.71), respectively.

After receiving the study intervention, the mean change from baseline (standard deviation) in total ASIA sensory score evaluated on D2, D3, D4, D7, D14, D28, and D56 for subjects in the 4800 mg group were 1.0 point (10.44), 10.7 points (25.40), 14.7 points (22.74), 13.3 points (34.02), 19.3 points (38.80), 30.7 points (27.59), and 37.3 points (22.48), respectively.

After receiving the study intervention, the mean change from baseline (standard deviation) in total ASIA sensory score evaluated on D2, D3, D4, D7, D14, D28, and D56 for subjects in the placebo group were 5.0 points (8.92), 7.4 points (19.10), 8.0 points (11.37), 12.0 points (12.82), 14.0 points (20.32), 20.6 points (28.63), and 52.3 points (35.52), respectively.

At the D56 visit, the mean change from baseline in the ASIA sensory total score for subjects in the 600 mg and 1200 mg groups was higher than that of the placebo group.

TABLE 11
Analysis of Changes from Baseline in ASIA Total Sensory Scores (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 156.5 (36.96) | 123.0 (54.98) | 157.0 (45.18) | 144.7 (21.20) | 148.7 (35.23) | 118.3 (47.22) | 137.4 (41.99) |
| | Deviation) | | | | | | | |
| | Median | 153.0 | 132.0 | 141.0 | 148.0 | 168.0 | 120.0 | 138.5 |
| | Quartiles (Q1, | 125.0, 188.0 | 88.0, 158.0 | 122.0, 208.0 | 122.0, 164.0 | 108.0, 170.0 | 58.0, 168.0 | 121.0, 168.0 |
| | Q3) | | | | | | | |
| | Minimum, | 124, 196 | 48, 180 | 122, 208 | 122, 164 | 108, 170 | 54, 168 | 48, 208 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 7.5 (5.97) | 3.0 (15.10) | 18.3 (22.19) | 27.3 (50.85) | 1.0 (10.44) | 5.0 (8.92) | 9.2 (20.53) |
| | Deviation) | | | | | | | |
| | Median | 8.0 | 0.0 | 12.0 | 0.0 | −4.0 | 0.0 | 0.0 |
| | Quartiles (Q1, | 3.0, 12.0 | −6.0, 12.0 | 0.0, 43.0 | −4.0, 86.0 | −6.0, 13.0 | 0.0, 8.0 | 0.0, 13.0 |
| | Q3) | | | | | | | |
| | Minimum, | 0, 14 | −12, 24 | 0, 43 | −4, 86 | −6, 13 | 0, 22 | −12, 86 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 10.5 (7.72) | 5.5 (19.82) | 35.3 (30.75) | 28.7 (53.15) | 10.7 (25.40) | 7.4 (19.10) | 14.2 (25.59) |
| | Deviation) | | | | | | | |
| | Median | 12.0 | 0.0 | 50.0 | 0.0 | −4.0 | 0.0 | 0.0 |
| | Quartiles (Q1, | 5.0, 16.0 | −6.0, 17.0 | 0.0, 56.0 | −4.0, 90.0 | −4.0, 40.0 | 0.0, 8.0 | 0.0, 26.0 |
| | Q3) | | | | | | | |
| | Minimum, | 0, 18 | −12, 34 | 0, 56 | −4, 90 | −4, 40 | −12, 48 | −12, 90 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 13.0 (10.65) | 8.5 (17.00) | 36.3 (28.22) | 28.7 (53.15) | 14.7 (22.74) | 8.0 (11.37) | 15.9 (23.50) |
| | Deviation) | | | | | | | |
| | Median | 13.0 | 0.0 | 49.0 | 0.0 | 8.0 | 2.0 | 6.0 |
| | Quartiles (Q1, | 6.0, 20.0 | 0.0, 17.0 | 4.0, 56.0 | −4.0, 90.0 | −4.0, 40.0 | 0.0, 16.0 | 0.0, 28.0 |
| | Q3) | | | | | | | |
| | Minimum, | 0, 26 | 0, 34 | 4, 56 | −4, 90 | −4, 40 | 0, 30 | −4, 90 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 37.0 (31.09) | 20.0 (40.13) | 46.7 (33.55) | 38.7 (49.17) | 13.3 (34.02) | 12.0 (12.82) | 25.3 (31.17) |
| | Deviation) | | | | | | | |
| | Median | 37.0 | 2.0 | 64.0 | 22.0 | 0.0 | 8.0 | 12.5 |
| | Quartiles (Q1, | 11.0, 63.0 | −2.0, 42.0 | 8.0, 68.0 | 0.0, 94.0 | −12.0, 52.0 | 0.0, 20.0 | 0.0, 54.0 |
| | Q3) | | | | | | | |
| | Minimum, | 4, 70 | −4, 80 | 8, 68 | 0, 94 | −12, 52 | 0, 35 | −12, 94 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 27.0 (37.26) | 32.0 (40.92) | 54.7 (36.95) | 38.7 (54.93) | 19.3 (38.80) | 14.0 (20.32) | 28.6 (35.01) |
| | Deviation) | | | | | | | |
| | Median | 11.0 | 18.0 | 76.0 | 10.0 | 0.0 | 11.0 | 14.0 |
| | Quartiles (Q1, | 4.0, 50.0 | 8.0, 56.0 | 12.0, 76.0 | 4.0, 102.0 | −6.0, 64.0 | 0.0, 24.0 | 4.0, 64.0 |
| | Q3) | | | | | | | |
| | Minimum, | 4, 82 | 0, 92 | 12, 76 | 4, 102 | −6, 64 | −10, 48 | −10, 102 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 33.5 (40.54) | 43.3 (36.25) | 61.3 (43.88) | 63.0 (41.15) | 30.7 (27.59) | 20.6 (28.63) | 38.2 (35.22) |
| | Deviation) | | | | | | | |
| | Median | 16.0 | 36.0 | 76.0 | 67.0 | 20.0 | 8.0 | 22.0 |
| | Quartiles (Q1, | 11.0, 56.0 | 16.5, 70.0 | 12.0, 96.0 | 20.0, 102.0 | 10.0, 62.0 | 0.0, 50.0 | 9.5, 67.5 |
| | Q3) | | | | | | | |
| | Minimum, | 8, 94 | 9, 92 | 12, 96 | 20, 102 | 10, 62 | −10, 68 | −10, 102 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 49.0 (32.60) | 77.5 (45.59) | 62.7 (41.63) | 44.0 (50.71) | 37.3 (22.48) | 52.3 (35.52) | 54.3 (36.30) |
| | Deviation) | | | | | | | |
| | Median | 43.0 | 70.0 | 76.0 | 22.0 | 32.0 | 56.0 | 56.0 |
| | Quartiles (Q1, | 29.0, 69.0 | 44.0, 111.0 | 16.0, 96.0 | 8.0, 102.0 | 18.0, 62.0 | 18.0, 80.0 | 20.0, 82.0 |
| | Q3) | | | | | | | |
| | Minimum, | 16, 94 | 32, 138 | 16, 96 | 8, 102 | 18, 62 | −8, 92 | −8, 138 |
| | Maximum | | | | | | | |

Note:
Time points are baseline, D 2, D 3, D 4, D 7, D 14, D 28, and D 56.

ii. ASIA Total Sensory Score—Left

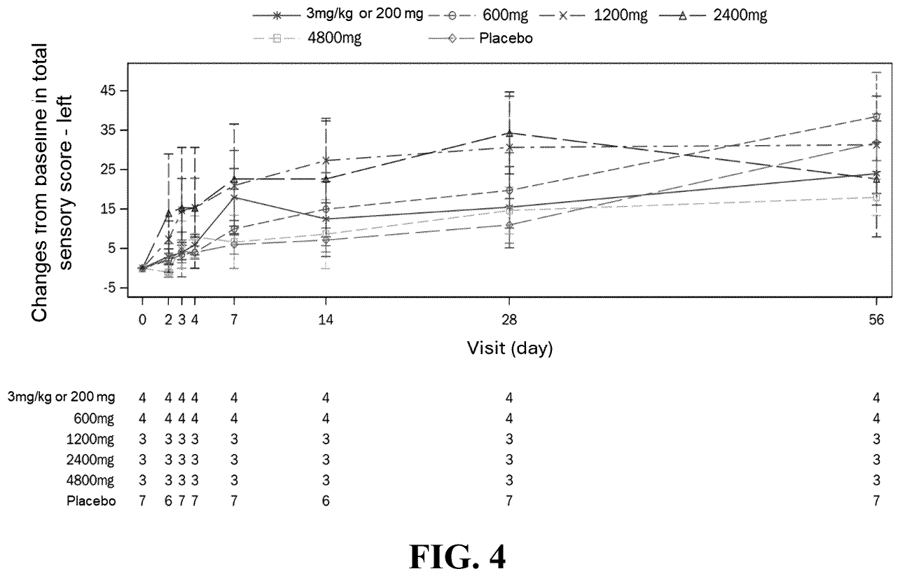

Based on FAS, the mean±SE time curve of changes from baseline in ASIA Total Sensory Score—Left is shown in FIG. 4. The analysis of changes from baseline in ASIA Total Sensory Score—Left is shown in Table 12.

After receiving the study intervention, the mean change (standard deviation) from baseline in total ASIA sensory score—left evaluated on D2, D3, D4, D7, D14, D28, and D56 for subjects in the 200 mg (or 3 mg/kg) group were 3.0 points (3.83), 4.0 points (3.65), 6.0 points (5.16), 18.0 points (14.61), 12.5 points (19.00), 15.5 points (20.62), and 24.0 points (15.92), respectively.

After receiving the study intervention, the mean change (standard deviation) from baseline in total ASIA sensory score—left evaluated on D2, D3, D4, D7, D14, D28, and D56 for subjects in the 600 mg group were 2.0 points (8.49), 3.5 points (11.36), 4.0 points (8.00), 10.0 points (20.07), 15.0 points (18.51), 19.8 points (19.12), and 38.5 points (22.35), respectively.

After receiving the study intervention, the mean change (standard deviation) from baseline in total ASIA sensory score—left evaluated on D2, D3, D4, D7, D14, D28, and D56 for subjects in the 1200 mg group were 7.3 points (8.08), 14.7 points (14.05), 15.3 points (13.01), 21.0 points (15.39), 27.3 points (18.58), 30.7 points (22.48), and 31.3 points (21.39), respectively.

After receiving the study intervention, the mean change (standard deviation) from baseline in total ASIA sensory score—left evaluated on D2, D3, D4, D7, D14, D28, and D56 for subjects in the 2400 mg group were 14.0 points (26.00), 15.3 points (26.56), 15.3 points (26.56), 22.7 points (24.11), 22.7 points (25.48), 34.3 points (18.01), and 22.7 points (25.48), respectively.

After receiving the study intervention, the mean change (standard deviation) from baseline in total ASIA sensory score—left evaluated on D2, D3, D4, D7, D14, D28, and D56 for subjects in the 4800 mg group were −1.0 points (1.00), 6.0 points (10.39), 8.0 points (9.17), 6.7 points (11.72), 8.7 points (15.14), 14.7 points (10.26), and 18.0 points (8.00), respectively.

After receiving the study intervention, the mean change (standard deviation) from baseline in total ASIA sensory score—left evaluated on D2, D3, D4, D7, D14, D28, and D56 for subjects in the placebo group were 2.3 points (3.67), 4.3 points (7.70), 4.0 points (4.47), 6.0 points (6.30), 7.2 points (7.44), 11.0 points (12.26), and 31.9 points (19.36), respectively.

At the D56 visit, the mean change from baseline in the left ASIA total sensory score for subjects in the 600 mg group was higher than that of the placebo group.

TABLE 12
Analysis of Change from Baseline in ASIA Total Sensory Score - Left (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 79.0 (17.63) | 62.0 (26.58) | 76.3 (24.83) | 71.3 (10.26) | 75.3 (13.32) | 55.3 (21.72) | 67.5 (20.68) |
| | Deviation) | | | | | | | |
| | Median | 77.0 | 66.0 | 69.0 | 74.0 | 82.0 | 60.0 | 66.0 |
| | Quartiles (Q1, | 64.0, 94.0 | 45.0, 79.0 | 56.0, | 60.0, 80.0 | 60.0, 84.0 | 27.0, 72.0 | 60.0, 83.0 |
| | Q3) | | | | | | | |
| | Minimum, | 64, 98 | 26, 90 | 56, 104 | 60, 80 | 60, 84 | 26, 84 | 26, 104 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 3.0 (3.83) | 2.0 (8.49) | 7.3 (8.08) | 14.0 (26.00) | −1.0 (1.00) | 2.3 (3.67) | 4.1 (10.13) |
| | Deviation) | | | | | | | |
| | Median | 2.0 | 0.0 | 6.0 | 0.0 | −1.0 | 0.0 | 0.0 |
| | Quartiles (Q1, | 0.0, 6.0 | −3.0, 7.0 | 0.0, 16.0 | −2.0, 44.0 | −2.0, 0.0 | 0.0, 6.0 | 0.0, 6.0 |
| | Q3) | | | | | | | |
| | Minimum, | 0, 8 | −6, 14 | 0, 16 | −2, 44 | −2, 0 | 0, 8 | −6, 44 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 4.0 (3.65) | 3.5 (11.36) | 14.7 (14.05) | 15.3 (26.56) | 6.0 (10.39) | 4.3 (7.70) | 7.0 (12.03) |
| | Deviation) | | | | | | | |
| | Median | 4.0 | 0.0 | 16.0 | 0.0 | 0.0 | 0.0 | 0.0 |
| | Quartiles (Q1, | 1.0, 7.0 | −3.0, 10.0 | 0.0, 28.0 | 0.0, 46.0 | 0.0, 18.0 | 0.0, 8.0 | 0.0, 12.0 |
| | Q3) | | | | | | | |
| | Minimum, | 0, 8 | −6, 20 | 0, 28 | 0, 46 | 0, 18 | −2, 20 | −6, 46 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 6.0 (5.16) | 4.0 (8.00) | 15.3 (13.01) | 15.3 (26.56) | 8.0 (9.17) | 4.0 (4.47) | 7.7 (11.08) |
| | Deviation) | | | | | | | |
| | Median | 6.0 | 0.0 | 16.0 | 0.0 | 6.0 | 2.0 | 3.0 |
| | Quartiles (Q1, | 2.0, 10.0 | 0.0, 8.0 | 2.0, 28.0 | 0.0, 46.0 | 0.0, 18.0 | 0.0, 8.0 | 0.0, 11.0 |
| | Q3) | | | | | | | |
| | Minimum, | 0, 12 | 0, 16 | 2, 28 | 0, 46 | 0, 18 | 0, 10 | 0, 46 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 18.0 (14.61) | 10.0 (20.07) | 21.0 (15.39) | 22.7 (24.11) | 6.7 (11.72) | 6.0 (6.30) | 12.7 (14.84) |
| | Deviation) | | | | | | | |
| | Median | 18.0 | 1.0 | 25.0 | 20.0 | 2.0 | 4.0 | 6.5 |
| | Quartiles (Q1, | 6.0, 30.0 | −1.0, 21.0 | 4.0, 34.0 | 0.0, 48.0 | −2.0, 20.0 | 0.0, 10.0 | 1.0, 22.5 |
| | Q3) | | | | | | | |
| | Minimum, | 2, 34 | −2, 40 | 4, 34 | 0, 48 | −2, 20 | 0, 17 | −2, 48 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 12.5 (19.00) | 15.0 (18.51) | 27.3 (18.58) | 22.7 (25.48) | 8.7 (15.14) | 7.2 (7.44) | 14.3 (16.47) |
| | Deviation) | | | | | | | |
| | Median | 6.0 | 9.0 | 36.0 | 10.0 | 2.0 | 4.0 | 8.0 |
| | Quartiles (Q1, | 0.0, 25.0 | 4.0, 26.0 | 6.0, 40.0 | 6.0, 52.0 | −2.0, 26.0 | 3.0, 12.0 | 2.0, 26.0 |
| | Q3) | | | | | | | |
| | Minimum, | −2, 40 | 0, 42 | 6, 40 | 6, 52 | −2, 26 | 0, 20 | −2, 52 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 15.5 (20.62) | 19.8 (19.12) | 30.7 (22.48) | 34.3 (18.01) | 14.7 (10.26) | 11.0 (12.26) | 19.0 (17.21) |
| | Deviation) | | | | | | | |
| | Median | 7.0 | 18.0 | 36.0 | 35. | 12.0 | 4.0 | 12.0 |
| | Quartiles (Q1, | 3.0, 28.0 | 5.5, 34.0 | 6.0, 50.0 | 16.0, 52.0 | 6.0, 26.0 | 2.0, 26.0 | 4.0, 32.5 |
| | Q3) | | | | | | | |
| | Minimum, | 2, 46 | −1, 44 | 6, 50 | 16, 52 | 6, 26 | 0, 30 | −1, 52 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 24.0 (15.92) | 38.5 (22.35) | 31.3 (21.39) | 22.7 (25.48) | 18.0 (8.00) | 31.9 (19.36) | 28.7 (18.49) |
| | Deviation) | | | | | | | |
| | Median | 21.0 | 35.0 | 36.0 | 10.0 | 18.0 | 32.0 | 28.0 |
| | Quartiles (Q1, | 14.0, 34.0 | 22.0, 55.0 | 8.0, 50.0 | 6.0, 52.0 | 10.0, 26.0 | 28.0, 46.0 | 13.0, 44.0 |
| | Q3) | | | | | | | |
| | Minimum, | 8, 46 | 16, 68 | 8, 50 | 6, 52 | 10, 26 | −6, 56 | −6, 68 |
| | Maximum | | | | | | | |

Note:
Time points are baseline, D 2, D 3, D 4, D 7, D 14, D 28, and D 56.

iii. ASIA Total Sensory Score—Right

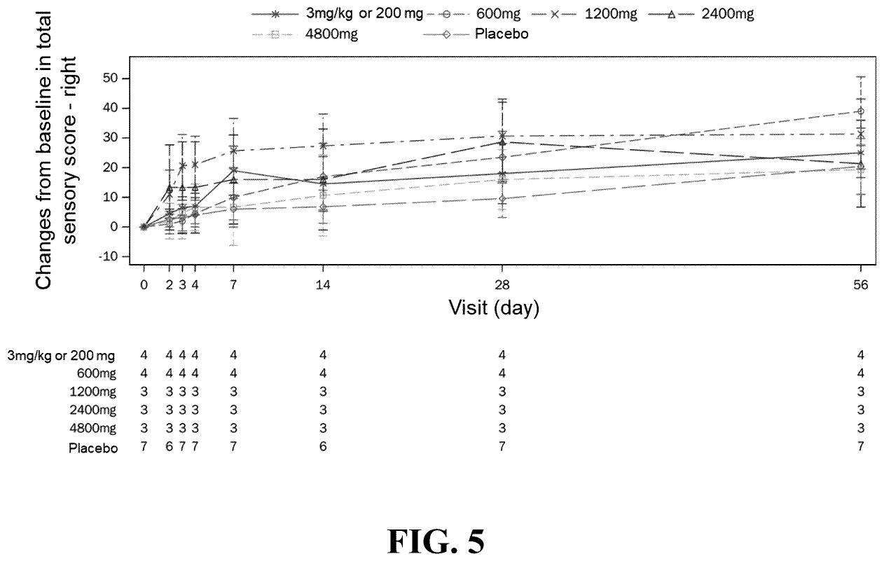

Based on FAS, the mean±SE time curve of change from baseline in ASIA total sensory score—right is shown in FIG. 5. Analysis of change from baseline in ASIA total sensory score—right is presented in Table 13.

After receiving the study intervention, the mean change (standard deviation) from baseline in total ASIA sensory score—right evaluated on D2, D3, D4, D7, D14, D28, and D56 for subjects in the 200 mg (or 3 mg/kg) group were 4.5 points (3.00), 6.5 points (5.00), 7.0 points (5.77), 19.0 points (16.53), 14.5 points (18.50), 18.0 points (20.26), and 25.0 points (16.69), respectively.

After receiving the study intervention, the mean change (standard deviation) from baseline in total ASIA sensory score—right evaluated on D2, D3, D4, D7, D14, D28, and D56 for subjects in the 600 mg group were 1.0 points (6.63), 2.0 points (8.49), 4.5 points (9.00), 10.0 points (20.07), 17.0 points (22.42), 23.5 points (17.46), and 39.0 points (23.24), respectively.

After receiving the study intervention, the mean change (standard deviation) from baseline in total ASIA sensory score—right evaluated on D2, D3, D4, D7, D14, D28, and D56 for subjects in the 1200 mg group were 11.0 points (14.18), 20.7 points (18.15), 21.0 points (16.64), 25.7 points (18.93), 27.3 points (18.58), 30.7 points (21.57), and 31.3 points (20.43), respectively.

After receiving the study intervention, the mean change (standard deviation) from baseline in total ASIA sensory score—right evaluated on D2, D3, D4, D7, D14, D28, and D56 for subjects in the 2400 mg group were 13.3 points (24.85), 13.3 points (26.63), 13.3 points (26.63), 16.0 points (26.00), 16.0 points (29.46), 28.7 points (23.18), and 21.3 points (25.32), respectively.

After receiving the study intervention, the mean change (standard deviation) from baseline in total ASIA sensory score—right evaluated on D2, D3, D4, D7, D14, D28, and D56 for subjects in the 4800 mg group were 2.0 points (10.39), 4.7 points (15.01), 6.7 points (13.61), 6.7 points (22.30), 10.7 points (23.69), 16.0 points (17.44), and 19.3 points (14.74), respectively.

After receiving the study intervention, the mean change (standard deviation) from baseline in total ASIA sensory score—right evaluated on D2, D3, D4, D7, D14, D28, and D56 for subjects in the placebo group were 2.7 points (6.53), 3.1 points (11.77), 4.0 points (7.66), 6.0 points (9.52), 6.8 points (13.66), 9.6 points (16.89), and 20.4 points (24.79), respectively.

At the D56 visit, the mean change from baseline in ASIA sensory total score—right score for subjects in the 200 mg (or 3 mg/kg) group, 600 mg group, 1200 mg, and 2400 mg groups was higher than that of the placebo group.

TABLE 13
Analysis of Change from Baseline in ASIA Total Sensory Score - Right (FAS).

| | | 3 mg/kg or | | | | | Placebo | |
|---|---|---|---|---|---|---|---|---|
| | | 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 77.5 (19.35) | 61.0 (28.40) | 80.7 (20.43) | 73.3 (11.02) | 73.3 (21.94) | 63.0 (29.64) | 69.9 (22.99) |
| | Deviation) | | | | | | | |
| | Median | 76.0 | 66.0 | 72.0 | 74.0 | 86.0 | 60.0 | 70.0 |
| | Quartiles (Q1, | 61.0, 94.0 | 43.0, 79.0 | 66.0, 104.0 | 62.0, 84.0 | 48.0, 86.0 | 32.0, 84.0 | 60.0, 86.0 |
| | Q3) | | | | | | | |
| | Minimum, | 60, 98 | 22, 90 | 66, 104 | 62, 84 | 48, 86 | 27, 112 | 22, 112 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 4.5 (3.00) | 1.0 (6.63) | 11.0 (14.18) | 13.3 (24.85) | 2.0 (10.39) | 2.7 (6.53) | 5.1 (11.01) |
| | Deviation) | | | | | | | |
| | Median | 6.0 | 0.0 | 6.0 | 0.0 | −4.0 | 0.0 | 0.0 |
| | Quartiles (Q1, | 3.0, 6.0 | −3.0, 5.0 | 0.0, 27.0 | −2.0, 42.0 | −4.0, 14.0 | 0.0, 0.0 | 0.0, 6.0 |
| | Q3) | | | | | | | |
| | Minimum, | 0, 6 | −6, 10 | 0, 27 | −2, 42 | −4, 14 | 0, 16 | −6, 42 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 6.5 (5.00) | 2.0 (8.49) | 20.7 (18.15) | 13.3 (26.63) | 4.7 (15.01) | 3.1 (11.77) | 7.2 (14.07) |
| | Deviation) | | | | | | | |
| | Median | 7.0 | 0.0 | 28.0 | 0.0 | −4.0 | 0.0 | 0.0 |
| | Quartiles (Q1, | 3.0, 10.0 | −3.0, 7.0 | 0.0, 34.0 | −4.0, 44.0 | −4.0, 22.0 | 0.0, 4.0 | 0.0, 13.0 |
| | Q3) | | | | | | | |
| | Minimum, | 0, 12 | −6, 14 | 0, 34 | −4, 44 | −4, 22 | −10, 28 | −10, 44 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 7.0 (5.77) | 4.5 (9.00) | 21.0 (16.64) | 13.3 (26.63) | 6.7 (13.61) | 4.0 (7.66) | 8.2 (12.85) |
| | Deviation) | | | | | | | |
| | Median | 7.0 | 0.0 | 28.0 | 0.0 | 2.0 | 0.0 | 1.0 |
| | Quartiles (Q1, | 3.0, 11.0 | 0.0, 9.0 | 2.0, 33.0 | −4.0, 44.0 | −4.0, 22.0 | 0.0, 8.0 | 0.0, 16.0 |
| | Q3) | | | | | | | |
| | Minimum, | 0, 14 | 0, 18 | 2, 33 | −4, 44 | −4, 22 | 0, 20 | −4, 44 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 19.0 (16.53) | 10.0 (20.07) | 25.7 (18.93) | 16.0 (26.00) | 6.7 (22.30) | 6.0 (9.52) | 12.6 (17.17) |
| | Deviation) | | | | | | | |
| | Median | 19.0 | 1.0 | 34.0 | 2.0 | −2.0 | 2.0 | 3.0 |
| | Quartiles (Q1, | 5.0, 33.0 | −1.0, 21.0 | 4.0, 39.0 | 0.0, 46.0 | −10.0, | 0.0, 10.0 | 0.0, 31.0 |
| | Q3) | | | | | | | |
| | Minimum, | 2, 36 | −2, 40 | 4, 39 | 0, 46 | −10, 32 | 0, 26 | −10, 46 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 14.5 (18.50) | 17.0 (22.42) | 27.3 (18.58) | 16.0 (29.46) | 10.7 (23.69) | 6.8 (13.66) | 14.3 (19.00) |
| | Deviation) | | | | | | | |
| | Median | 7.0 | 9.0 | 36.0 | 0.0 | −2.0 | 7.0 | 8.0 |
| | Quartiles (Q1, | 4.0, 25.0 | 4.0, 30.0 | 6.0, 40.0 | −2.0, 50.0 | −4.0, 38.0 | 0.0, 12.0 | 0.0, 36.0 |
| | Q3) | | | | | | | |
| | Minimum, | 2, 42 | 0, 50 | 6, 40 | −2, 50 | −4, 38 | −13, 28 | −13, 50 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 18.0 (20.26) | 23.5 (17.46) | 30.7 (21.57) | 28.7 (23.18) | 16.0 (17.44) | 9.6 (16.89) | 19.1 (18.53) |
| | Deviation) | | | | | | | |
| | Median | 10.0 | 18.0 | 40.0 | 32.0 | 8.0 | 4.0 | 12.0 |
| | Quartiles (Q1, | 6.0, 30.0 | 11.0, 36.0 | 6.0, 46.0 | 4.0, 50.0 | 4.0, 36.0 | 0.0, 24.0 | 4.0, 37.0 |
| | Q3) | | | | | | | |
| | Minimum, | 4, 48 | 10, 48 | 6, 46 | 4, 50 | 4, 36 | −13, 38 | −13, 50 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 25.0 (16.69) | 39.0 (23.24) | 31.3 (20.43) | 21.3 (25.32) | 19.3 (14.74) | 20.4 (24.79) | 25.6 (20.72) |
| | Deviation) | | | | | | | |
| | Median | 22.0 | 35.0 | 40.0 | 12.0 | 14.0 | 28.0 | 25.0 |
| | Quartiles (Q1, | 15.0, 35.0 | 22.0, 56.0 | 8.0, 46.0 | 2.0, 50.0 | 8.0, 36.0 | −2.0, 44.0 | 8.0, 43.0 |
| | Q3) | | | | | | | |
| | Minimum, | 8, 48 | 16, 70 | 8, 46 | 2, 50 | 8, 36 | −13, 46 | −13, 70 |
| | Maximum | | | | | | | |

Note:
Time points are baseline, D 2, D 3, D 4, D 7, D 14, D 28, and D 56.

iv. Total Pinprick Score

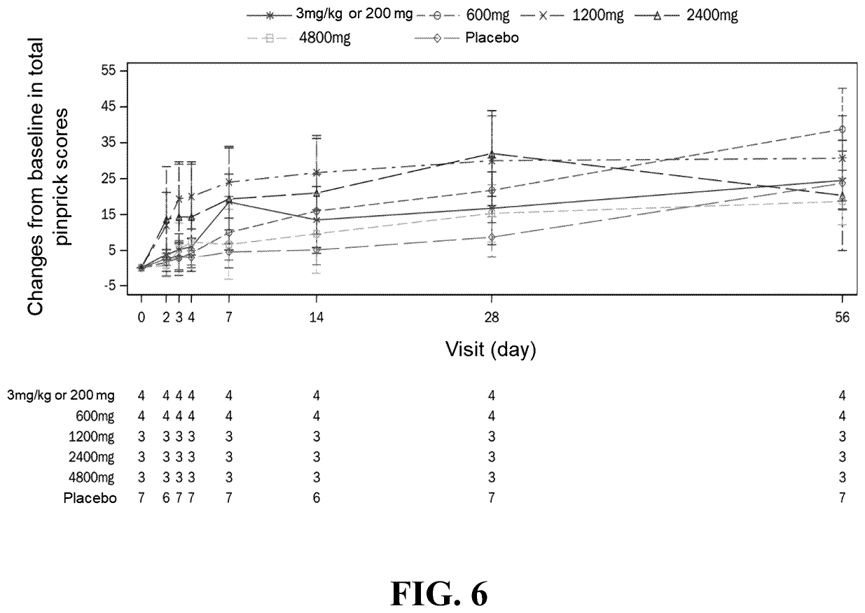

Based on FAS, the mean±SE time curve of change from baseline in total pinprick score is shown in FIG. 6. The analysis of change from baseline in total pinprick score is shown in Table 14.

After receiving the study intervention, the mean change from baseline (standard deviation) in total pinprick score evaluated on D2, D3, D4, D7, D14, D28, and D56 for subjects in the 200 mg (or 3 mg/kg) group were 3.8 points (2.99), 5.3 points (3.86), 6.0 points (4.55), 18.5 points (15.55), 13.5 points (18.63), 16.8 points (20.27), and 24.5 points (16.30), respectively.

After receiving the study intervention, the mean changes (standard deviations) from baseline in total pinprick scores evaluated on D2, D3, D4, D7, D14, D28, and D56 for subjects in the 600 mg group were 1.5 points (7.55), 2.8 points (9.91), 4.3 points (8.50), 10.0 points (20.07), 16.0 points (20.46), 21.8 points (17.97), and 38.8 points (22.79), respectively.

After receiving the study intervention, the mean changes (standard deviations) from baseline in total pinprick scores evaluated on D2, D3, D4, D7, D14, D28, and D56 for subjects in the 1200 mg group were 12.0 points (15.87), 19.3 points (16.77), 20.0 points (15.62), 24.0 points (17.32), 26.7 points (17.93), 30.0 points (21.63), and 30.7 points (20.53), respectively.

After receiving the study intervention, the mean changes (standard deviations) from baseline in total pinprick scores evaluated on D2, D3, D4, D7, D14, D28, and D56 for subjects in the 2400 mg group were 13.7 points (25.42), 14.3 points (26.58), 14.3 points (26.58), 19.3 points (24.58), 21.0 points (26.29), 32.0 points (20.66), and 20.3 points (26.58), respectively.

After receiving the study intervention, the mean changes (standard deviations) from baseline in total pinprick scores evaluated on D2, D3, D4, D7, D14, D28, and D56 for subjects in the 4800 mg group were 0.7 points (5.51), 5.3 points (12.70), 7.3 points (11.37), 6.7 points (17.01), 9.7 points (19.40), 15.3 points (13.80), and 18.7 points (11.24), respectively.

After receiving the study intervention, the mean changes (standard deviations) from baseline in total pinprick scores evaluated on D2, D3, D4, D7, D14, D28, and D56 for subjects in the placebo group were 2.5 points (4.46), 3.1 points (9.65), 2.9 points (5.55), 4.6 points (6.37), 5.2 points (10.59), 8.7 points (15.00), and 23.7 points (18.74), respectively.

At the D56 visit, the mean changes from baseline in total pinprick sensation scores for subjects in the 200 mg (or 3 mg/kg) group, 600 mg group, and 1200 mg group were higher than those in the placebo group.

TABLE 14
Analysis of Changes from Baseline in Total Pinprick Scores (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 78.3 (18.48) | 61.5 (27.49) | 80.3 (21.83) | 72.3 (10.60) | 74.3 (17.62) | 61.6 (25.86) | 69.6 (21.42) |
| | Deviation) | | | | | | | |
| | Median | 76.5 | 66.0 | 76.0 | 74.0 | 84.0 | 60.0 | 70.0 |
| | Quartiles (Q1, | 62.5, 94.0 | 44.0, 79.0 | 61.0, | 61.0, 82.0 | 54.0, 85.0 | 30.0, 84.0 | 60.5, 84.5 |
| | Q3) | | | | | | | |
| | Minimum, | 62, 98 | 24, 90 | 61, 104 | 61, 82 | 54, 85 | 29, 98 | 24, 104 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 3.8 (2.99) | 1.5 (7.55) | 12.0 (15.87) | 13.7 (25.42) | 0.7 (5.51) | 2.5 (4.46) | 5.0 (11.03) |
| | Deviation) | | | | | | | |
| | Median | 4.0 | 0.0 | 6.0 | 0.0 | −2.0 | 0.0 | 0.0 |
| | Quartiles (Q1, | 1.5, 6.0 | −3.0, 6.0 | 0.0, 30.0 | −2.0, 43.0 | −3.0, 7.0 | 0.0, 4.0 | 0.0, 7.0 |
| | Q3) | | | | | | | |
| | Minimum, | 0, 7 | −6, 12 | 0, 30 | −2, 43 | −3, 7 | 0, 11 | −6, 43 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 5.3 (3.86) | 2.8 (9.91) | 19.3 (16.77) | 14.3 (26.58) | 5.3 (12.70) | 3.1 (9.65) | 7.1 (13.21) |
| | Deviation) | | | | | | | |
| | Median | 6.0 | 0.0 | 28.0 | 0.0 | −2.0 | 0.0 | 0.0 |
| | Quartiles (Q1, | 2.5, 8.0 | −3.0, 8.5 | 0.0, 30.0 | −2.0, 45.0 | −2.0, 20.0 | 0.0, 4.0 | 0.0, 13.0 |
| | Q3) | | | | | | | |
| | Minimum, | 0, 9 | −6, 17 | 0, 30 | −2, 45 | −2, 20 | −6, 24 | −6, 45 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 6.0 (4.55) | 4.3 (8.50) | 20.0 (15.62) | 14.3 (26.58) | 7.3 (11.37) | 2.9 (5.55) | 7.8 (12.22) |
| | Deviation) | | | | | | | |
| | Median | 6.5 | 0.0 | 28.0 | 0.0 | 4.0 | 0.0 | 1.5 |
| | Quartiles (Q1, | 3.0, 9.0 | 0.0, 8.5 | 2.0, 30.0 | −2.0, 45.0 | −2.0, 20.0 | 0.0, 4.0 | 0.0, 13.0 |
| | Q3) | | | | | | | |
| | Minimum, | 0, 11 | 0, 17 | 2, 30 | −2, 45 | −2, 20 | 0, 15 | −2, 45 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 18.5 (15.55) | 10.0 (20.07) | 24.0 (17.32) | 19.3 (24.58) | 6.7 (17.01) | 4.6 (6.37) | 12.3 (15.90) |
| | Deviation) | | | | | | | |
| | Median | 18.5 | 1.0 | 34.0 | 11.0 | 0.0 | 2.0 | 4.0 |
| | Quartiles (Q1, | 5.5, 31.5 | −1.0, 21.0 | 4.0, 34.0 | 0.0, 47.0 | −6.0, 26.0 | 0.0, 9.0 | 0.0, 27.0 |
| | Q3) | | | | | | | |
| | Minimum, | 2, 35 | −2, 40 | 4, 34 | 0, 47 | −6, 26 | 0, 17 | −6, 47 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 13.5 (18.63) | 16.0 (20.46) | 26.7 (17.93) | 21.0 (26.29) | 9.7 (19.40) | 5.2 (10.59) | 14.0 (17.59) |
| | Deviation) | | | | | | | |
| | Median | 5.5 | 9.0 | 36.0 | 10.0 | 0.0 | 4.0 | 7.0 |
| | Quartiles (Q1, | 2.0, 25.0 | 4.0, 28.0 | 6.0, 38.0 | 2.0, 51.0 | −3.0, 32.0 | 0.0, 7.0 | 2.0, 32.0 |
| | Q3) | | | | | | | |
| | Minimum, | 2, 41 | 0, 46 | 6, 38 | 2, 51 | −3, 32 | −8, 24 | −8, 51 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 16.8 (20.27) | 21.8 (17.97) | 30.0 (21.63) | 32.0 (20.66) | 15.3 (13.80) | 8.7 (15.00) | 18.6 (17.96) |
| | Deviation) | | | | | | | |
| | Median | 8.0 | 18.0 | 36.0 | 35.0 | 10.0 | 4.0 | 10.0 |
| | Quartiles (Q1, | 5.5, 28.0 | 8.5, 35.0 | 6.0, 48.0 | 10.0, 51.0 | 5.0, 31.0 | 0.0, 25.0 | 4.5, 34.5 |
| | Q3) | | | | | | | |
| | Minimum, | 4, 47 | 5, 46 | 6, 48 | 10, 51 | 5, 31 | −8, 34 | −8, 51 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 24.5 (16.30) | 38.8 (22.79) | 30.7 (20.53) | 20.3 (26.58) | 18.7 (11.24) | 23.7 (18.74) | 26.2 (18.64) |
| | Deviation) | | | | | | | |
| | Median | 21.5 | 35.0 | 36.0 | 6.0 | 16.0 | 28.0 | 25.0 |
| | Quartiles (Q1, | 14.5, 34.5 | 22.0, 55.5 | 8.0, 48.0 | 4.0, 51.0 | 9.0, 31.0 | 6.0, 40.0 | 8.5, 41.0 |
| | Q3) | | | | | | | |
| | Minimum, | 8, 47 | 16, 69 | 8, 48 | 4, 51 | 9, 31 | −4, 46 | −4, 69 |
| | Maximum | | | | | | | |

Note:
Time points are baseline, D 2, D 3, D 4, D 7, D 14, D 28, and D 56.

v. Total Light Touch Score

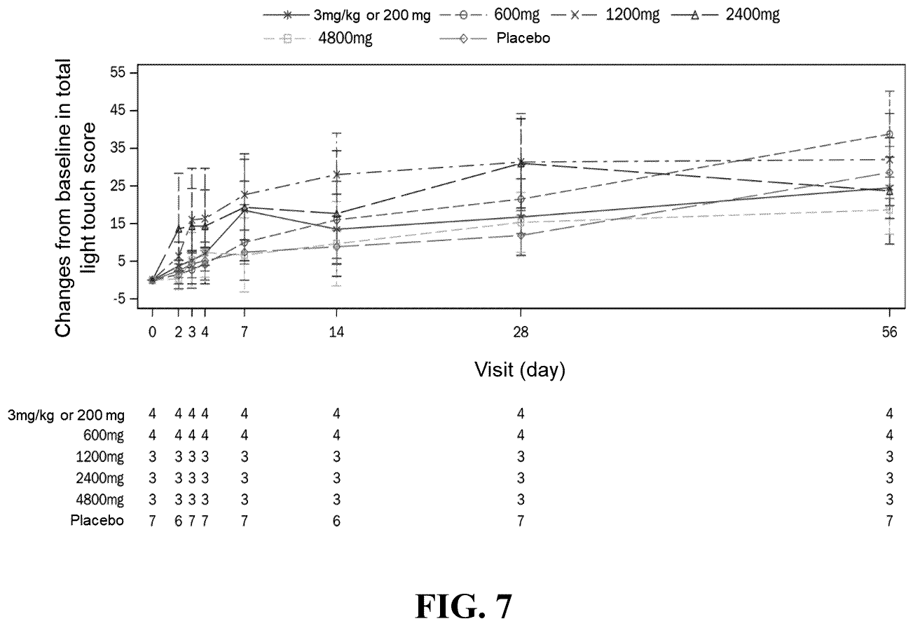

Based on FAS, the mean±SE time curve of changes from baseline in total light touch scores is shown in FIG. 7. The analysis of changes from baseline in total light touch scores is presented in Table 15.

After receiving the study intervention, the mean change from baseline (standard deviation) in total light touch score evaluated on D2, D3, D4, D7, D14, D28, and D56 for subjects in the 200 mg (or 3 mg/kg) group were 3.8 points (2.99), 5.3 points (3.86), 7.0 points (6.16), 18.5 points (15.55), 13.5 points (18.63), 16.8 points (20.27), and 24.5 points (16.30), respectively.

After receiving the study intervention, the mean changes (standard deviations) from baseline in total light touch scores evaluated on D2, D3, D4, D7, D14, D28, and D56 for subjects in the 600 mg group were 1.5 points (7.55), 2.8 points (9.91), 4.3 points (8.50), 10.0 points (20.07), 16.0 points (20.46), 21.5 points (18.28), and 38.8 points (22.79), respectively.

After receiving the study intervention, the mean changes (standard deviations) from baseline in total light touch scores evaluated on D2, D3, D4, D7, D14, D28, and D56 for subjects in the 1200 mg group were 6.3 points (6.51), 16.0 points (14.42), 16.3 points (13.20), 22.7 points (16.29), 28.0 points (19.08), 31.3 points (22.30), and 32.0 points (21.17), respectively.

After receiving the study intervention, the mean changes (standard deviations) from baseline in total light touch scores evaluated on D2, D3, D4, D7, D14, D28, and D56 for subjects in the 2400 mg group were 13.7 points (25.42), 14.3 points (26.58), 14.3 points (26.58), 19.3 points (24.58), 17.7 points (28.88), 31.0 points (20.52), and 23.7 points (24.42), respectively.

After receiving the study intervention, the mean changes (standard deviations) from baseline in total light touch scores evaluated on D2, D3, D4, D7, D14, D28, and D56 for subjects in the 4800 mg group were 0.3 points (4.93), 5.3 points (12.70), 7.3 points (11.37), 6.7 points (17.01), 9.7 points (19.40), 15.3 points (13.80), and 18.7 points (11.24), respectively.

After receiving the study intervention, the mean changes (standard deviations) from baseline in total light touch scores evaluated on D2, D3, D4, D7, D14, D28, and D56 for subjects in the placebo group were 2.5 points (4.46), 4.3 points (9.69), 5.1 points (7.22), 7.4 points (8.38), 8.8 points (10.74), 11.9 points (14.26), and 28.6 points (18.28), respectively.

At the D56 visit, the mean change from baseline in total light touch scores for subjects in the 600 mg and 1200 mg groups was higher than that of the placebo group.

TABLE 15
Analysis of Changes from Baseline in Total Light Touch Score (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 78.3 (18.48) | 61.5 (27.49) | 76.7 (23.76) | 72.3 (10.60) | 74.3 (17.62) | 56.7 (22.38) | 67.8 (21.01) |
| | Deviation) | | | | | | | |
| | Median | 76.5 | 66.0 | 65.0 | 74.0 | 84.0 | 60.0 | 66.5 |
| | Quartiles (Q1, | 62.5, 94.0 | 44.0, 79.0 | 61.0, | 61.0, 82.0 | 54.0, 85.0 | 29.0, 72.0 | 60.5, 84.0 |
| | Q3) | | | | | | | |
| | Minimum, | 62, 98 | 24, 90 | 61, 104 | 61, 82 | 54, 85 | 24, 84 | 24, 104 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 3.8 (2.99) | 1.5 (7.55) | 6.3 (6.51) | 13.7 (25.42) | 0.3 (4.93) | 2.5 (4.46) | 4.2 (9.77) |
| | Deviation) | | | | | | | |
| | Median | 4.0 | 0.0 | 6.0 | 0.0 | −2.0 | 0.0 | 0.0 |
| | Quartiles (Q1, | 1.5, 6.0 | −3.0, 6.0 | 0.0, 13.0 | −2.0, 43.0 | −3.0, 6.0 | 0.0, 4.0 | 0.0, 6.0 |
| | Q3) | | | | | | | |
| | Minimum, | 0, 7 | −6, 12 | 0, 13 | −2, 43 | −3, 6 | 0, 11 | −6, 43 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 5.3 (3.86) | 2.8 (9.91) | 16.0 (14.42) | 14.3 (26.58) | 5.3 (12.70) | 4.3 (9.69) | 7.0 (12.52) |
| | Deviation) | | | | | | | |
| | Median | 6.0 | 0.0 | 20.0 | 0.0 | −2.0 | 0.0 | 0.0 |
| | Quartiles (Q1, | 2.5, 8.0 | −3.0, 8.5 | 0.0, 28.0 | −2.0, 45.0 | −2.0, 20.0 | 0.0, 8.0 | 0.0, 13.0 |
| | Q3) | | | | | | | |
| | Minimum, | 0, 9 | −6, 17 | 0, 28 | −2, 45 | −2, 20 | −6, 24 | −6, 45 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 7.0 (6.16) | 4.3 (8.50) | 16.3 (13.20) | 14.3 (26.58) | 7.3 (11.37) | 5.1 (7.22) | 8.1 (11.63) |
| | Deviation) | | | | | | | |
| | Median | 6.5 | 0.0 | 19.0 | 0.0 | 4.0 | 1.0 | 3.0 |
| | Quartiles (Q1, | 3.0, 11.0 | 0.0, 8.5 | 2.0, 28.0 | −2.0, 45.0 | −2.0, 20.0 | 0.0, 15.0 | 0.0, 15.5 |
| | Q3) | | | | | | | |
| | Minimum, | 0, 15 | 0, 17 | 2, 28 | −2, 45 | −2, 20 | 0, 16 | −2, 45 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 18.5 (15.55) | 10.0 (20.07) | 22.7 (16.29) | 19.3 (24.58) | 6.7 (17.01) | 7.4 (8.38) | 13.0 (15.56) |
| | Deviation) | | | | | | | |
| | Median | 18.5 | 1.0 | 30.0 | 11.0 | 0.0 | 4.0 | 6.0 |
| | Quartiles (Q1, | 5.5, 31.5 | −1.0, 21.0 | 4.0, 34.0 | 0.0, 47.0 | −6.0, 26.0 | 0.0, 18.0 | 0.0, 27.0 |
| | Q3) | | | | | | | |
| | Minimum, | 2, 35 | −2, 40 | 4, 34 | 0, 47 | −6, 26 | 0, 20 | −6, 47 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 13.5 (18.63) | 16.0 (20.46) | 28.0 (19.08) | 17.7 (28.88) | 9.7 (19.40) | 8.8 (10.74) | 14.7 (17.68) |
| | Deviation) | | | | | | | |
| | Median | 5.5 | 9.0 | 38.0 | 2.0 | 0.0 | 5.5 | 7.0 |
| | Quartiles (Q1, | 2.0, 25.0 | 4.0, 28.0 | 6.0, 40.0 | 0.0, 51.0 | −3.0, 32.0 | 0.0, 20.0 | 0.0, 32.0 |
| | Q3) | | | | | | | |
| | Minimum, | 2, 41 | 0, 46 | 6, 40 | 0, 51 | −3, 32 | −2, 24 | −3, 51 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 16.8 (20.27) | 21.5 (18.28) | 31.3 (22.30) | 31.0 (20.52) | 15.3 (13.80) | 11.9 (14.26) | 19.5 (17.44) |
| | Deviation) | | | | | | | |
| | Median | 8.0 | 18.0 | 40.0 | 32.0 | 10.0 | 4.0 | 11.0 |
| | Quartiles (Q1, | 5.5, 28.0 | 8.0, 35.0 | 6.0, 48.0 | 10.0, 51.0 | 5.0, 31.0 | 0.0, 25.0 | 4.5, 33.0 |
| | Q3) | | | | | | | |
| | Minimum, | 4, 47 | 4, 46 | 6, 48 | 10, 51 | 5, 31 | −2, 34 | −2, 51 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 24.5 (16.30) | 38.8 (22.79) | 32.0 (21.17) | 23.7 (24.42) | 18.7 (11.24) | 28.6 (18.28) | 28.2 (18.16) |
| | Deviation) | | | | | | | |
| | Median | 21.5 | 35.0 | 40.0 | 16.0 | 16.0 | 36.0 | 28.0 |
| | Quartiles (Q1, | 14.5, 34.5 | 22.0, 55.5 | 8.0, 48.0 | 4.0, 51.0 | 9.0, 31.0 | 12.0, 42.0 | 14.0, 42.0 |
| | Q3) | | | | | | | |
| | Minimum, | 8, 47 | 16, 69 | 8, 48 | 4, 51 | 9, 31 | −4, 46 | −4, 69 |
| | Maximum | | | | | | | |

Note:
Time points are baseline, D 2, D 3, D 4, D 7, D 14, D 28, and D 56.

b. change from Baseline in ASIA Sensory Score
i. Total Motor Score

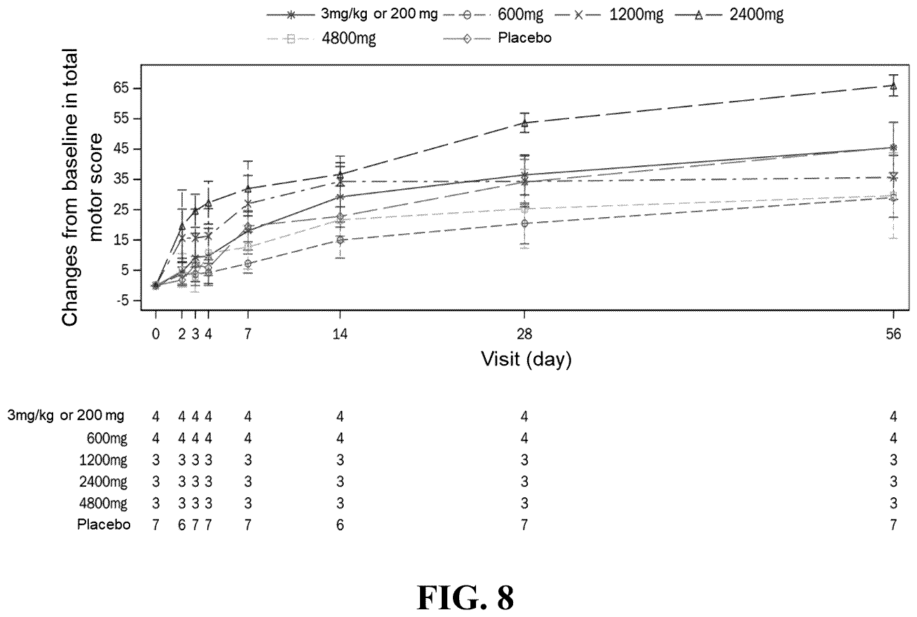

Based on FAS, the mean±SE time curve of changes from baseline in total motor score is shown in FIG. 8. The analysis of changes from baseline in total motor score is presented in Table 16.

After receiving the study intervention, the mean change from baseline (standard deviation) in total motor score evaluated on D2, D3, D4, D7, D14, D28, and D56 for subjects in the 200 mg (or 3 mg/kg) group were 4.5 points (9.00), 9.3 points (15.95), 9.8 points (18.17), 18.0 points (12.57), 29.3 points (19.96), 36.5 points (13.20), and 45.5 points (16.60), respectively.

After receiving the study intervention, the mean changes (standard deviations) from baseline in total motor scores evaluated on D2, D3, D4, D7, D14, D28, and D56 for subjects in the 600 mg group were 3.8 points (7.50), 3.8 points (7.50), 4.3 points (8.50), 7.3 points (6.29), 15.0 points (11.86), 20.5 points (13.48), and 29.0 points (12.94), respectively.

After receiving the study intervention, the mean changes (standard deviations) from baseline in total motor scores evaluated on D2, D3, D4, D7, D14, D28, and D56 for subjects in the 1200 mg group were 15.7 points (16.56), 15.7 points (16.56), 16.3 points (15.63), 27.0 points (16.09), 34.3 points (14.50), 34.3 points (14.50), and 35.7 points (12.58), respectively.

After receiving the study intervention, the mean changes (standard deviations) from baseline in total motor scores evaluated on D2, D3, D4, D7, D14, D28, and D56 for subjects in the 2400 mg group were 19.7 points (20.55), 24.7 points (9.45), 27.3 points (12.22), 32.0 points (15.62), 36.7 points (6.66), 53.7 points (5.51), and 66.0 points (6.00), respectively.

After receiving the study intervention, the mean changes (standard deviations) from baseline in total motor scores evaluated on D2, D3, D4, D7, D14, D28, and D56 for subjects in the 4800 mg group were 5.0 points (9.54), 3.0 points (8.89), 10.7 points (11.59), 12.7 points (12.50), 21.7 points (12.66), 25.3 points (22.55), and 29.7 points (24.44), respectively.

After receiving the study intervention, the mean changes (standard deviations) from baseline in total motor scores evaluated on D2, D3, D4, D7, D14, D28, and D56 for subjects in the placebo group were 1.8 points (3.25), 6.9 points (7.29), 5.9 points (6.84), 19.6 points (13.61), 22.8 points (15.99), 34.1 points (19.73), and 45.6 points (22.14), respectively.

At the D56 visit, the mean change from baseline in the total motor score for subjects in the 2400 mg group was higher than that of the placebo group.

TABLE 16
Analysis of Changes from Baseline in Total Motor Score (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 44.5 (20.74) | 27.8 (18.52) | 54.3 (18.93) | 27.7 (10.69) | 19.3 (14.64) | 22.1 (18.60) | 31.2 (19.96) |
| | Deviation) | | | | | | | |
| | Median | 52.0 | 36.5 | 46.0 | 30.0 | 17.0 | 20.0 | 32.5 |
| | Quartiles (Q1, | 32.0, 57.0 | 18.0, 37.5 | 41.0, 76.0 | 16.0, 37.0 | 6.0, 35.0 | 9.0, 28.0 | 15.0, 43.5 |
| | Q3) | | | | | | | |
| | Minimum, | 14, 60 | 0, 38 | 41, 76 | 16, 37 | 6, 35 | 4, 60 | 0, 76 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 4.5 (9.00) | 3.8 (7.50) | 15.7 (16.56) | 19.7 (20.55) | 5.0 (9.54) | 1.8 (3.25) | 7.2 (11.65) |
| | Deviation) | | | | | | | |
| | Median | 0.0 | 0.0 | 14.0 | 18.0 | 0.0 | 0.0 | 0.0 |
| | Quartiles (Q1, | 0.0, 9.0 | 0.0, 7.5 | 0.0, 33.0 | 0.0, 41.0 | −1.0, 16.0 | 0.0, 3.0 | 0.0, 15.0 |
| | Q3) | | | | | | | |
| | Minimum, | 0, 18 | 0, 15 | 0, 33 | 0, 41 | −1, 16 | 0, 8 | −1, 41 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 9.3 (15.95) | 3.8 (7.50) | 15.7 (16.56) | 24.7 (9.45) | 3.0 (8.89) | 6.9 (7.29) | 9.6 (11.88) |
| | Deviation) | | | | | | | |
| | Median | 2.0 | 0.0 | 14.0 | 28.0 | 0.0 | 8.0 | 6.0 |
| | Quartiles (Q1, | 0.0, 18.5 | 0.0, 7.5 | 0.0, 33.0 | 14.0, 32.0 | −4.0, 13.0 | 0.0, 14.0 | 0.0, 14.5 |
| | Q3) | | | | | | | |
| | Minimum, | 0, 33 | 0, 15 | 0, 33 | 14, 32 | −4, 13 | 0, 18 | −4, 33 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 9.8 (18.17) | 4.3 (8.50) | 16.3 (15.63) | 27.3 (12.22) | 10.7 (11.59) | 5.9 (6.84) | 10.8 (12.86) |
| | Deviation) | | | | | | | |
| | Median | 1.0 | 0.0 | 14.0 | 30.0 | 9.0 | 2.0 | 5.0 |
| | Quartiles (Q1, | 0.5, 19.0 | 0.0, 8.5 | 2.0, 33.0 | 14.0, 38.0 | 0.0, 23.0 | 0.0, 14.0 | 0.0, 16.5 |
| | Q3) | | | | | | | |
| | Minimum, | 0, 37 | 0, 17 | 2, 33 | 14, 38 | 0, 23 | 0, 16 | 0, 38 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 18.0 (12.57) | 7.3 (6.29) | 27.0 (16.09) | 32.0 (15.62) | 12.7 (12.50) | 19.6 (13.61) | 18.9 (13.83) |
| | Deviation) | | | | | | | |
| | Median | 21.5 | 8.0 | 29.0 | 40.0 | 13.0 | 12.0 | 13.5 |
| | Quartiles (Q1, | 8.5, 27.5 | 2.0, 12.5 | 10.0, 42.0 | 14.0, 42.0 | 0.0, 25.0 | 8.0, 36.0 | 9.0, 28.5 |
| | Q3) | | | | | | | |
| | Minimum, | 1, 28 | 0, 13 | 10, 42 | 14, 42 | 0, 25 | 5, 38 | 0, 42 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 29.3 (19.96) | 15.0 (11.86) | 34.3 (14.50) | 36.7 (6.66) | 21.7 (12.66) | 22.8 (15.99) | 25.7 (14.95) |
| | Deviation) | | | | | | | |
| | Median | 35.0 | 17.5 | 34.0 | 40.0 | 24.0 | 25.5 | 29.0 |
| | Quartiles (Q1, | 15.5, 43.0 | 5.5, 24.5 | 20.0, 49.0 | 29.0, 41.0 | 8.0, 33.0 | 9.0, 38.0 | 11.0, 39.0 |
| | Q3) | | | | | | | |
| | Minimum, | 1, 46 | 0, 25 | 20, 49 | 29, 41 | 8, 33 | 0, 39 | 0, 49 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 36.5 (13.20) | 20.5 (13.48) | 34.3 (14.50) | 53.7 (5.51) | 25.3 (22.55) | 34.1 (19.73) | 33.6 (17.49) |
| | Deviation) | | | | | | | |
| | Median | 36.0 | 24.5 | 34.0 | 54.0 | 27.0 | 39.0 | 32.5 |
| | Quartiles (Q1, | 26.0, 47.0 | 10.5, 30.5 | 20.0, 49.0 | 48.0, 59.0 | 2.0, 47.0 | 19.0, 56.0 | 21.0, 48.5 |
| | Q3) | | | | | | | |
| | Minimum, | 22, 52 | 2, 31 | 20, 49 | 48, 59 | 2, 47 | 4, 58 | 2, 59 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 45.5 (16.60) | 29.0 (12.94) | 35.7 (12.58) | 66.0 (6.00) | 29.7 (24.44) | 45.6 (22.14) | 42.1 (19.75) |
| | Deviation) | | | | | | | |
| | Median | 47.0 | 33.5 | 34.0 | 66.0 | 35.0 | 58.0 | 46.5 |
| | Quartiles (Q1, | 34.0, 57.0 | 21.5, 36.5 | 24.0, 49.0 | 60.0, 72.0 | 3.0, 51.0 | 21.0, 60.0 | 28.5, 60.0 |
| | Q3) | | | | | | | |
| | Minimum, | 24, 64 | 10, 39 | 24, 49 | 60, 72 | 3, 51 | 7, 61 | 3, 72 |
| | Maximum | | | | | | | |

Note:
Time points are baseline, D 2, D 3, D 4, D 7, D 14, D 28, and D 56.

ii. Total Motor Score—Left

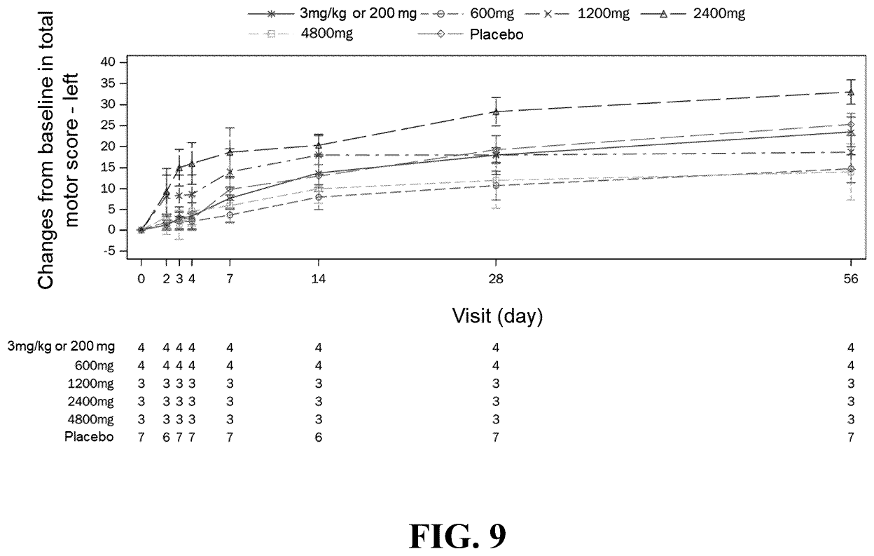

Based on FAS, the mean±SE time curve of changes from baseline in the Total Motor Score—Left is shown in FIG. 9. The analysis of changes from baseline in the Total Motor Score—Left is provided in Table 17.

After receiving the study intervention, the mean change from baseline (standard deviation) in total motor score—left evaluated on D2, D3, D4, D7, D14, D28, and D56 for subjects in the 200 mg (or 3 mg/kg) group were 1.3 points (2.50), 3.0 points (5.35), 3.5 points (6.35), 7.8 points (5.32), 13.8 points (8.77), 18.0 points (3.46), and 23.5 points (7.05), respectively.

After receiving the study intervention, the mean change from baseline (standard deviation) in total motor score—left evaluated on D2, D3, D4, D7, D14, D28, and D56 for subjects in the 600 mg group were 2.0 points (4.00), 2.3 points (4.50), 3.8 points (3.30), 8.0 points (5.94), 10.8 points (6.85), and 14.8 points (6.65), respectively.

After receiving the study intervention, the mean change from baseline (standard deviation) in total motor score—left evaluated on D2, D3, D4, D7, D14, D28, and D56 for subjects in the 1200 mg group were 8.3 points (8.50), 8.3 points (8.50), 8.7 points (8.02), 14.0 points (9.54), 18.0 points (8.00), and 18.7 points (7.02), respectively.

After receiving the study intervention, the mean change from baseline (standard deviation) in total motor score—left evaluated on D2, D3, D4, D7, D14, D28, and D56 for subjects in the 2400 mg group were 9.3 points (9.50), 15.0 points (7.55), 16.0 points (8.54), 18.7 points (10.07), 20.3 points (4.51), 28.3 points (5.86), and 33.0 points (5.0), respectively.

After receiving the study intervention, the mean change from baseline (standard deviation) in total motor score—left evaluated on D2, D3, D4, D7, D14, D28, and D56 for subjects in the 4800 mg group were 3.3 points (7.57), 1.3 points (6.11), 4.7 points (6.43), 6.0 points (7.21), 10.0 points (6.00), 12.0 points (11.53), and 14.0 points (11.53), respectively.

After receiving the study intervention, the mean change from baseline (standard deviation) in total motor score—left evaluated on D2, D3, D4, D7, D14, D28, and D56 for subjects in the placebo group were 1.2 points (1.83), 3.1 points (3.18), 2.7 points (3.59), 9.9 points (7.03), 13.0 points (6.69), 19.3 points (8.85), and 25.3 points (7.04), respectively.

At the D56 visit, the mean change from baseline in the total motor score—left side score for subjects in the 2400 mg group was higher than that of the placebo group.

TABLE 17
Analysis of Changes from Baseline in Total Motor Score - Left (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 20.8 (13.33) | 13.8 (9.18) | 26.0 (10.58) | 15.7 (6.03) | 10.7 (8.62) | 8.3 (4.92) | 14.7 (9.89) |
| | Deviation) | | | | | | | |
| | Median | 26.0 | 18.0 | 22.0 | 15.0 | 9.0 | 10.0 | 15.5 |
| | Quartiles (Q1, | 13.0, 28.5 | 9.0, 18.5 | 18.0, 38.0 | 10.0, 22.0 | 3.0, 20.0 | 3.0, 11.0 | 7.5, 21.0 |
| | Q3) | | | | | | | |
| | Minimum, | 1, 30 | 0, 19 | 18, 38 | 10, 22 | 3, 20 | 2, 16 | 0, 38 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 1.3 (2.50) | 2.0 (4.00) | 8.3 (8.50) | 9.3 (9.50) | 3.3 (7.57) | 1.2 (1.83) | 3.6 (5.87) |
| | Deviation) | | | | | | | |
| | Median | 0.0 | 0.0 | 8.0 | 9.0 | 0.0 | 0.0 | 0.0 |
| | Quartiles (Q1, | 0.0, 2.5 | 0.0, 4.0 | 0.0, 17.0 | 0.0, 19.0 | −2.0, 12.0 | 0.0, 3.0 | 0.0, 8.0 |
| | Q3) | | | | | | | |
| | Minimum, | 0, 5 | 0, 8 | 0, 17 | 0, 19 | −2, 12 | 0, 4 | −2, 19 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 3.0 (5.35) | 2.3 (4.50) | 8.3 (8.50) | 15.0 (7.55) | 1.3 (6.11) | 3.1 (3.18) | 4.9 (6.54) |
| | Deviation) | | | | | | | |
| | Median | 0.5 | 0.0 | 8.0 | 14.0 | 0.0 | 4.0 | 2.5 |
| | Quartiles (Q1, | 0.0, 6.0 | 0.0, 4.5 | 0.0, 17.0 | 8.0, 23.0 | −4.0, 8.0 | 0.0, 7.0 | 0.0, 8.0 |
| | Q3) | | | | | | | |
| | Minimum, | 0, 11 | 0, 9 | 0, 17 | 8, 23 | −4, 8 | 0, 7 | −4, 23 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 3.5 (6.35) | 2.3 (4.50) | 8.7 (8.02) | 16.0 (8.54) | 4.7 (6.43) | 2.7 (3.59) | 5.4 (6.89) |
| | Deviation) | | | | | | | |
| | Median | 0.5 | 0.0 | 8.0 | 15.0 | 2.0 | 0.0 | 1.5 |
| | Quartiles (Q1, | 0.0, 7.0 | 0.0, 4.5 | 1.0, 17.0 | 8.0, 25.0 | 0.0, 12.0 | 0.0, 7.0 | 0.0, 8.5 |
| | Q3) | | | | | | | |
| | Minimum, | 0, 13 | 0, 9 | 1, 17 | 8, 25 | 0, 12 | 0, 8 | 0, 25 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 7.8 (5.32) | 3.8 (3.30) | 14.0 (9.54) | 18.7 (10.07) | 6.0 (7.21) | 9.9 (7.03) | 9.6 (7.80) |
| | Deviation) | | | | | | | |
| | Median | 8.0 | 4.0 | 13.0 | 20.0 | 4.0 | 6.0 | 7.5 |
| | Quartiles (Q1, | 4.5, 11.0 | 1.0, 6.5 | 5.0, 24.0 | 8.0, 28.0 | 0.0, 14.0 | 4.0, 19.0 | 4.0, 14.0 |
| | Q3) | | | | | | | |
| | Minimum, | 1, 14 | 0, 7 | 5, 24 | 8, 28 | 0, 14 | 3, 19 | 0, 28 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 13.8 (8.77) | 8.0 (5.94) | 18.0 (8.00) | 20.3 (4.51) | 10.0 (6.00) | 13.0 (6.69) | 13.5 (7.27) |
| | Deviation) | | | | | | | |
| | Median | 17.0 | 9.5 | 18.0 | 20.0 | 10.0 | 13.0 | 15.0 |
| | Quartiles (Q1, | 8.0, 19.5 | 3.5, 12.5 | 10.0, 26.0 | 16.0, 25.0 | 4.0, 16.0 | 9.0, 19.0 | 9.0, 19.0 |
| | Q3) | | | | | | | |
| | Minimum, | 1, 20 | 0, 13 | 10, 26 | 16, 25 | 4, 16 | 3, 21 | 0, 26 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 18.0 (3.46) | 10.8 (6.85) | 18.0 (8.00) | 28.3 (5.86) | 12.0 (11.53) | 19.3 (8.85) | 17.7 (8.74) |
| | Deviation) | | | | | | | |
| | Median | 18.0 | 13.0 | 18.0 | 26.0 | 11.0 | 20.0 | 18.5 |
| | Quartiles (Q1, | 15.0, 21.0 | 6.0, 15.5 | 10.0, 26.0 | 24.0, 35.0 | 1.0, 24.0 | 9.0, 29.0 | 11.0, 24.0 |
| | Q3) | | | | | | | |
| | Minimum, | 15, 21 | 1, 16 | 10, 26 | 24, 35 | 1, 24 | 7, 30 | 1, 35 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 23.5 (7.05) | 14.8 (6.65) | 18.7 (7.02) | 33.0 (5.00) | 14.0 (11.53) | 25.3 (7.04) | 22.0 (9.03) |
| | Deviation) | | | | | | | |
| | Median | 23.5 | 17.0 | 18.0 | 33.0 | 15.0 | 29.0 | 23.5 |
| | Quartiles (Q1, | 18.5, 28.5 | 11.0, 18.5 | 12.0, 26.0 | 28.0, 38.0 | 2.0, 25.0 | 21.0, 30.0 | 16.0, 29.0 |
| | Q3) | | | | | | | |
| | Minimum, | 15, 32 | 5, 20 | 12, 26 | 28, 38 | 2, 25 | 11, 30 | 2, 38 |
| | Maximum | | | | | | | |

Note:
Time points are baseline, D 2, D 3, D 4, D 7, D 14, D 28, and D 56.

iii. Total Motor Score—Right

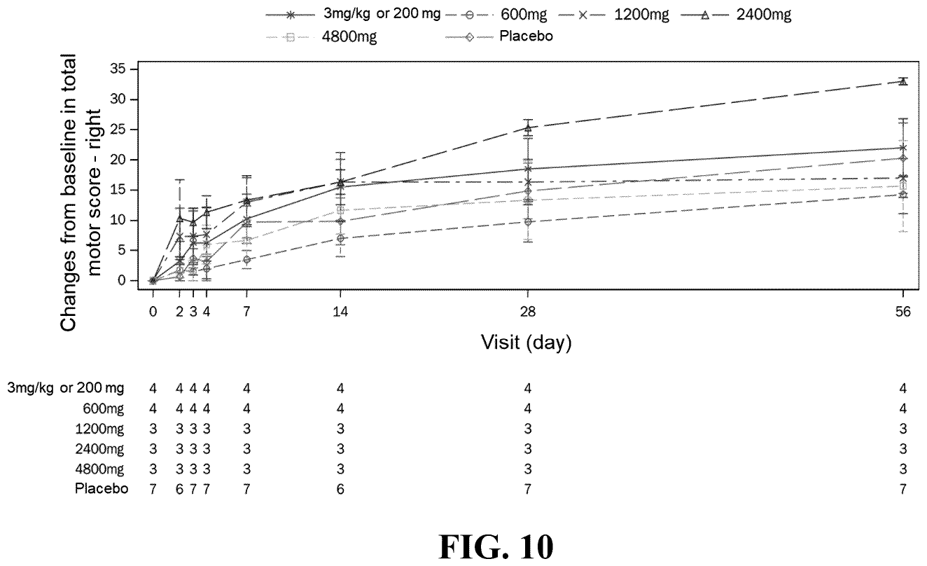

Based on FAS, the mean±SE time curve of changes from baseline in the Total Motor Score—Right is shown in FIG. 10. The analysis of changes from baseline in Total Motor Score—Right is presented in Table 18.

After receiving the study intervention, the mean change from baseline (standard deviation) in total motor score—right evaluated on D2, D3, D4, D7, D14, D28, and D56 for subjects in the 200 mg (or 3 mg/kg) group were 3.3 points (6.50), 6.3 points (10.59), 6.3 points (11.84), 10.3 points (8.18), 15.5 points (11.45), 18.5 points (10.12), and 22.0 points (9.63), respectively.

After receiving the study intervention, the mean change from baseline (standard deviation) in total motor score—right evaluated on D2, D3, D4, D7, D14, D28, and D56 for subjects in the 600 mg group were 1.8 points (3.50), 1.5 points (3.00), 2.0 points (4.00), 3.5 points (3.00), 7.0 points (6.00), 9.8 points (6.70), and 14.3 points (6.29), respectively.

After receiving the study intervention, the mean change from baseline (standard deviation) in total motor score—right evaluated on D2, D3, D4, D7, D14, D28, and D56 for subjects in the 1200 mg/kg or 200 mg were 7.3 points (8.08), 7.3 points (8.08), 7.7 points (7.64), 13.0 points (7.00), 16.3 points (6.51), 16.3 points (6.51), and 17.0 points (5.57), respectively.

After receiving the study intervention, the mean change from baseline (standard deviation) in total motor score—right evaluated on D2, D3, D4, D7, D14, D28, and D56 for subjects in the 2400 mg group were 10.3 points (11.06), 9.7 points (4.04), 11.3 points (4.73), 13.3 points (7.02), 16.3 points (3.51), 25.3 points (2.31), and 33.0 points (1.00), respectively.

After receiving the study intervention, the mean change from baseline (standard deviation) in total motor score—right evaluated on D2, D3, D4, D7, D14, D28, and D56 for subjects in the 4800 mg group were 1.7 points (2.08), 1.7 points (2.89), 6.0 points (5.57), 6.7 points (5.86), 11.7 points (6.81), 13.3 points (11.24), and 15.7 points (13.05), respectively.

After receiving the study intervention, the mean change from baseline (standard deviation) in total motor score—right evaluated on D2, D3, D4, D7, D14, D28, and D56 for subjects in the placebo group were 0.7 points (1.63), 3.7 points (4.19), 3.1 points (3.29), 9.7 points (6.97), 9.8 points (9.50), 14.9 points (12.19), and 20.3 points (15.41), respectively.

At the D56 visit, the mean change from baseline in the Total Motor Score−right side score for subjects in the 200 mg (or 3 mg/kg) group and the 2400 mg group was higher than that of the placebo group.

TABLE 18
Analysis of Changes from Baseline in Total Motor Score - Right (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean (Standard | 23.8 (7.46) | 14.0 (9.35) | 28.3 (8.39) | 12.0 (5.20) | 8.7 (6.03) | 13.9 (16.31) | 16.5 (11.87) |
| | Deviation) | | | | | | | |
| | Median | 26.0 | 18.5 | 24.0 | 15.0 | 8.0 | 10.0 | 15.0 |
| | Quartiles (Q1, | 19.0, 28.5 | 9.0, 19.0 | 23.0, 38.0 | 6.0, 15.0 | 3.0, 15.0 | 6.0, 12.0 | 7.0, 23.5 |
| | Q3) | | | | | | | |
| | Minimum, | 13, 30 | 0, 19 | 23, 38 | 6, 15 | 3, 15 | 2, 50 | 0, 50 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean (Standard | 3.3 (6.50) | 1.8 (3.50) | 7.3 (8.08) | 10.3 (11.06) | 1.7 (2.08) | 0.7 (1.63) | 3.6 (6.09) |
| | Deviation) | | | | | | | |
| | Median | 0.0 | 0.0 | 6.0 | 9.0 | 1.0 | 0.0 | 0.0 |
| | Quartiles (Q1, | 0.0, 6.5 | 0.0, 3.5 | 0.0, 16.0 | 0.0, 22.0 | 0.0, 4.0 | 0.0, 0.0 | 0.0, 6.0 |
| | Q3) | | | | | | | |
| | Minimum, | 0, 13 | 0, 7 | 0, 16 | 0, 22 | 0, 4 | 0, 4 | 0, 22 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean (Standard | 6.3 (10.59) | 1.5 (3.00) | 7.3 (8.08) | 9.7 (4.04) | 1.7 (2.89) | 3.7 (4.19) | 4.7 (6.00) |
| | Deviation) | | | | | | | |
| | Median | 1.5 | 0.0 | 6.0 | 9.0 | 0.0 | 4.0 | 3.5 |
| | Quartiles (Q1, | 0.0, 12.5 | 0.0, 3.0 | 0.0, 16.0 | 6.0, 14.0 | 0.0, 5.0 | 0.0, 7.0 | 0.0, 6.5 |
| | Q3) | | | | | | | |
| | Minimum, | 0, 22 | 0, 6 | 0, 16 | 6, 14 | 0, 5 | 0, 11 | 0, 22 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean (Standard | 6.3 (11.84) | 2.0 (4.00) | 7.7 (7.64) | 11.3 (4.73) | 6.0 (5.57) | 3.1 (3.29) | 5.4 (6.47) |
| | Deviation) | | | | | | | |
| | Median | 0.5 | 0.0 | 6.0 | 13.0 | 7.0 | 2.0 | 3.0 |
| | Quartiles (Q1, | 0.0, 12.5 | 0.0, 4.0 | 1.0, 16.0 | 6.0, 15.0 | 0.0, 11.0 | 0.0, 7.0 | 0.0, 8.0 |
| | Q3) | | | | | | | |
| | Minimum, | 0, 24 | 0, 8 | 1, 16 | 6, 15 | 0, 11 | 0, 8 | 0, 24 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean (Standard | 10.3 (8.18) | 3.5 (3.00) | 13.0 (7.00) | 13.3 (7.02) | 6.7 (5.86) | 9.7 (6.97) | 9.3 (6.71) |
| | Deviation) | | | | | | | |
| | Median | 11.0 | 4.0 | 16.0 | 14.0 | 9.0 | 9.0 | 8.5 |
| | Quartiles (Q1, | 4.0, 16.5 | 1.0, 6.0 | 5.0, 18.0 | 6.0, 20.0 | 0.0, 11.0 | 4.0, 17.0 | 4.5, 15.0 |
| | Q3) | | | | | | | |
| | Minimum, | 0, 19 | 0, 6 | 5, 18 | 6, 20 | 0, 11 | 0, 19 | 0, 20 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean (Standard | 15.5 (11.45) | 7.0 (6.00) | 16.3 (6.51) | 16.3 (3.51) | 11.7 (6.81) | 9.8 (9.50) | 12.3 (8.10) |
| | Deviation) | | | | | | | |
| | Median | 17.5 | 8.0 | 16.0 | 16.0 | 14.0 | 12.5 | 14.0 |
| | Quartiles (Q1, | 7.5, 23.5 | 2.0, 12.0 | 10.0, 23.0 | 13.0, 20.0 | 4.0, 17.0 | 0.0, 18.0 | 4.0, 18.0 |
| | Q3) | | | | | | | |
| | Minimum, | 0, 27 | 0, 12 | 10, 23 | 13, 20 | 4, 17 | −3, 19 | −3, 27 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean (Standard | 18.5 (10.12) | 9.8 (6.70) | 16.3 (6.51) | 25.3 (2.31) | 13.3 (11.24) | 14.9 (12.19) | 15.9 (9.68) |
| | Deviation) | | | | | | | |
| | Median | 18.0 | 11.5 | 16.0 | 24.0 | 16.0 | 18.0 | 16.0 |
| | Quartiles (Q1, | 11.0, 26.0 | 4.5, 15.0 | 10.0, 23.0 | 24.0, 28.0 | 1.0, 23.0 | 0.0, 26.0 | 9.0, 23.5 |
| | Q3) | | | | | | | |
| | Minimum, | 7, 31 | 1, 15 | 10, 23 | 24, 28 | 1, 23 | −3, 29 | −3, 31 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean (Standard | 22.0 (9.63) | 14.3 (6.29) | 17.0 (5.57) | 33.0 (1.00) | 15.7 (13.05) | 20.3 (15.41) | 20.2 (11.35) |
| | Deviation) | | | | | | | |
| | Median | 23.5 | 16.5 | 16.0 | 33.0 | 20.0 | 29.0 | 22.5 |
| | Quartiles (Q1, | 15.5, 28.5 | 10.5, 18.0 | 12.0, 23.0 | 32.0, 34.0 | 1.0, 26.0 | 0.0, 30.0 | 14.0, 30.0 |
| | Q3) | | | | | | | |
| | Minimum, | 9, 32 | 5, 19 | 12, 23 | 32, 34 | 1, 26 | −4, 32 | −4, 34 |
| | Maximum | | | | | | | |

Note:
Time points are baseline, D 2, D 3, D 4, D 7, D 14, D 28, and D 56.

iv. Upper Limb Motor Score

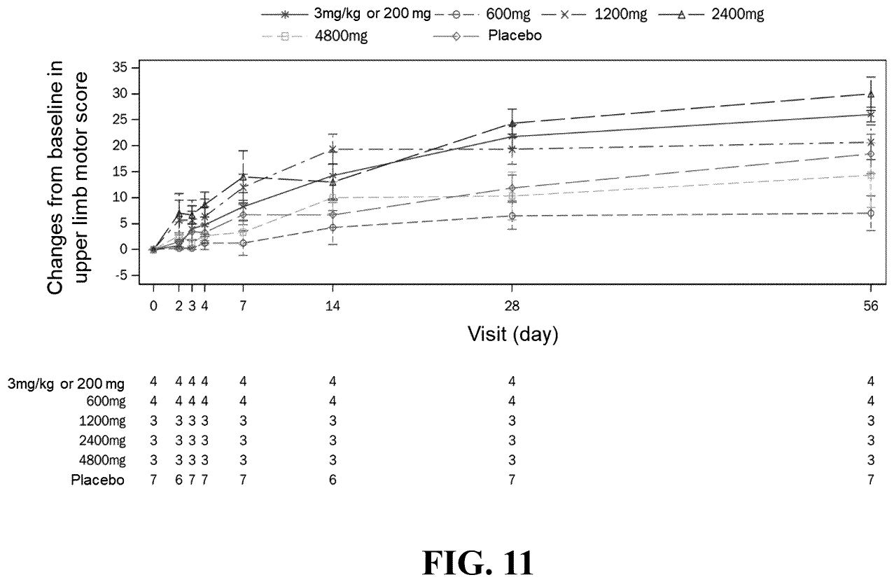

Based on FAS, the time curve of mean±SE changes from baseline in the upper limb motor score is shown in FIG. 11. The analysis of changes from baseline in the upper limb motor score is presented in Table 19.

After receiving the study intervention, the mean change from baseline (standard deviation) in upper limb motor score evaluated on D2, D3, D4, D7, D14, D28, and D56 for subjects in the 200 mg (or 3 mg/kg) group were 0.8 points (1.50), 4.0 points (6.73), 4.8 points (6.95), 8.3 points (5.44), 14.3 points (9.03), 21.8 points (4.50), and 26.0 points (2.83), respectively.

After receiving the study intervention, the mean change from baseline (standard deviation) in upper limb motor score evaluated on D2, D3, D4, D7, D14, D28, and D56 for subjects in the 600 mg group were 0.3 points (0.50), 0.3 points (0.50), 1.3 points (2.50), 1.3 points (4.72), 4.3 points (6.55), 6.5 points (5.20), and 7.0 points (6.68), respectively.

After receiving the study intervention, the mean changes (standard deviations) from baseline in upper limb motor score evaluated on D2, D3, D4, D7, D14, D28, and D56 for subjects in the 1200 mg group were 5.7 points (6.66), 5.7 points (6.66), 6.3 points (5.86), 12.0 points (4.36), 19.3 points (5.03), 19.3 points (5.03), and 20.7 points (5.77), respectively.

After receiving the study intervention, the mean changes (standard deviations) from baseline in upper limb motor score evaluated on D2, D3, D4, D7, D14, D28, and D56 for subjects in the 2400 mg group were 7.0 points (6.56), 6.7 points (3.06), 8.7 points (4.16), 14.0 points (8.72), 13.0 points (6.08), 24.3 points (4.73), and 30.0 points (5.57), respectively.

After receiving the study intervention, the mean changes (standard deviations) from baseline in upper limb motor score evaluated on D2, D3, D4, D7, D14, D28, and D56 for subjects in the 4800 mg group were 2.7 points (4.62), 1.3 points (1.15), 2.7 points (3.06), 3.3 points (4.16), 10.0 points (5.29), 10.3 points (8.02), and 14.3 points (10.79), respectively.

After receiving the study intervention, the mean change from baseline (standard deviation) in upper limb motor score evaluated on D2, D3, D4, D7, D14, D28, and D56 for subjects in the placebo group were 1.5 points (3.21), 3.4 points (4.12), 3.3 points (4.19), 6.7 points (5.12), 6.7 points (5.85), 11.9 points (6.59), and 18.4 points (10.05), respectively.

At the D56 visit, the mean change from baseline in upper limb motor scores for subjects in the 200 mg (or 3 mg/kg) group, the 1200 mg group, and the 2400 mg group was higher than that of the placebo group.

TABLE 19
Analysis of Changes from Baseline in Upper Limb Motor Score (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean | 15.3 (10.50) | 22.3 (17.75) | 22.7 (5.77) | 15.3 (6.11) | 10.7 (5.03) | 14.0 (9.78) | 16.4 (10.31) |
| | (Standard | | | | | | | |
| | Deviation) | | | | | | | |
| | Median | 17.0 | 25.5 | 26.0 | 14.0 | 10.0 | 12.0 | 16.0 |
| | Quartiles | 6.5, 24.0 | 8.0, 36.5 | 16.0, 26.0 | 10.0, 22.0 | 6.0, 16.0 | 7.0, 20.0 | 8.5, 24.0 |
| | (Q1, Q3) | | | | | | | |
| | Minimum, | 3, 24 | 0, 38 | 16, 26 | 10, 22 | 6, 16 | 2, 30 | 0, 38 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean | 0.8 (1.50) | 0.3 (0.50) | 5.7 (6.66) | 7.0 (6.56) | 2.7 (4.62) | 1.5 (3.21) | 2.6 (4.29) |
| | (Standard | | | | | | | |
| | Deviation) | | | | | | | |
| | Median | 0.0 | 0.0 | 4.0 | 8.0 | 0.0 | 0.0 | 0.0 |
| | Quartiles | 0.0, 1.5 | 0.0, 0.5 | 0.0, 13.0 | 0.0, 13.0 | 0.0, 8.0 | 0.0, 1.0 | 0.0, 4.0 |
| | (Q1, Q3) | | | | | | | |
| | Minimum, | 0, 3 | 0, 1 | 0, 13 | 0, 13 | 0, 8 | 0, 8 | 0, 13 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean | 4.0 (6.73) | 0.3 (0.50) | 5.7 (6.66) | 6.7 (3.06) | 1.3 (1.15) | 3.4 (4.12) | 3.4 (4.42) |
| | (Standard | | | | | | | |
| | Deviation) | | | | | | | |
| | Median | 1.0 | 0.0 | 4.0 | 6.0 | 2.0 | 2.0 | 2.0 |
| | Quartiles | 0.0, 8.0 | 0.0, 0.5 | 0.0, 13.0 | 4.0, 10.0 | 0.0, 2.0 | 0.0, 8.0 | 0.0, 5.0 |
| | (Q1, Q3) | | | | | | | |
| | Minimum, | 0, 14 | 0, 1 | 0, 13 | 4, 10 | 0, 2 | 0, 10 | 0, 14 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean | 4.8 (6.95) | 1.3 (2.50) | 6.3 (5.86) | 8.7 (4.16) | 2.7 (3.06) | 3.3 (4.19) | 4.2 (4.72) |
| | (Standard | | | | | | | |
| | Deviation) | | | | | | | |
| | Median | 2.0 | 0.0 | 4.0 | 10.0 | 2.0 | 1.0 | 2.5 |
| | Quartiles | 0.5, 9.0 | 0.0, 2.5 | 2.0, 13.0 | 4.0, 12.0 | 0.0, 6.0 | 0.0, 8.0 | 0.0, 7.0 |
| | (Q1, Q3) | | | | | | | |
| | Minimum, | 0, 15 | 0, 5 | 2, 13 | 4, 12 | 0, 6 | 0, 10 | 0, 15 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean | 8.3 (5.44) | 1.3 (4.72) | 12.0 (4.36) | 14.0 (8.72) | 3.3 (4.16) | 6.7 (5.12) | 7.2 (6.39) |
| | (Standard | | | | | | | |
| | Deviation) | | | | | | | |
| | Median | 9.0 | 0.0 | 10.0 | 18.0 | 2.0 | 8.0 | 8.0 |
| | Quartiles | 4.5, 12.0 | −1.5, 4.0 | 9.0, 17.0 | 4.0, 20.0 | 0.0, 8.0 | 2.0, 10.0 | 1.5, 10.0 |
| | (Q1, Q3) | | | | | | | |
| | Minimum, | 1, 14 | −3, 8 | 9, 17 | 4, 20 | 0, 8 | 0, 14 | −3, 20 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean | 14.3 (9.03) | 4.3 (6.55) | 19.3 (5.03) | 13.0 (6.08) | 10.0 (5.29) | 6.7 (5.85) | 10.5 (7.63) |
| | (Standard | | | | | | | |
| | Deviation) | | | | | | | |
| | Median | 18.0 | 1.5 | 20.0 | 10.0 | 8.0 | 7.5 | 10.0 |
| | Quartiles | 8.5, 20.0 | 0.5, 8.0 | 14.0, 24.0 | 9.0, 20.0 | 6.0, 16.0 | 0.0, 12.0 | 2.0, 16.0 |
| | (Q1, Q3) | | | | | | | |
| | Minimum, | 1, 20 | 0, 14 | 14, 24 | 9, 20 | 6, 16 | 0, 13 | 0, 24 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean | 21.8 (4.50) | 6.5 (5.20) | 19.3 (5.03) | 24.3 (4.73) | 10.3 (8.02) | 11.9 (6.59) | 14.9 (8.21) |
| | (Standard | | | | | | | |
| | Deviation) | | | | | | | |
| | Median | 22.0 | 5.0 | 20.0 | 26.0 | 11.0 | 12.0 | 15.0 |
| | Quartiles | 19.0, 24.5 | 3.5, 9.5 | 14.0, 24.0 | 19.0, 28.0 | 2.0, 18.0 | 4.0, 20.0 | 7.5, 21.0 |
| | (Q1, Q3) | | | | | | | |
| | Minimum, | 16, 27 | 2, 14 | 14, 24 | 19, 28 | 2, 18 | 4, 20 | 2, 28 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean | 26.0 (2.83) | 7.0 (6.68) | 20.7 (5.77) | 30.0 (5.57) | 14.3 (10.79) | 18.4 (10.05) | 19.0 (10.08) |
| | (Standard | | | | | | | |
| | Deviation) | | | | | | | |
| | Median | 25.0 | 6.0 | 24.0 | 29.0 | 19.0 | 20.0 | 21.0 |
| | Quartiles | 24.0, 28.0 | 2.5, 11.5 | 14.0, 24.0 | 25.0, 36.0 | 2.0, 22.0 | 7.0, 28.0 | 10.5, 25.5 |
| | (Q1, Q3) | | | | | | | |
| | Minimum, | 24, 30 | 0, 16 | 14, 24 | 25, 36 | 2, 22 | 6, 33 | 0, 36 |
| | Maximum | | | | | | | |

Note:
Upper limb motor score (sum of C5-T1 scores); time points were baseline, D 2, D 3, D 4, D 7, D 14, D 28, D 56.

v. Lower Limb Motor Score

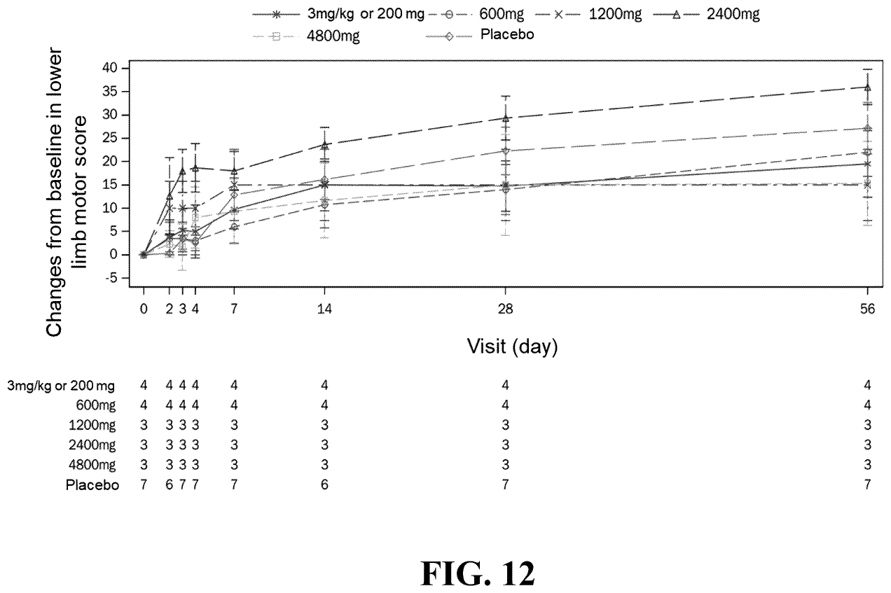

Based on FAS, the mean±SE time curve of changes from baseline in lower limb motor scores is shown in FIG. 12. The analysis of changes from baseline in upper limb motor scores is presented in Table 20.

After receiving the study intervention, the mean change from baseline (standard deviation) in lower limb motor score evaluated on D2, D3, D4, D7, D14, D28, and D56 for subjects in the 200 mg (or 3 mg/kg) group were 3.8 points (7.50), 5.3 points (9.22), 5.0 points (11.37), 9.8 points (8.42), 15.0 points (11.14), 14.8 points (10.81), and 19.5 points (14.27), respectively.

After receiving the study intervention, the mean change from baseline (standard deviation) in lower limb motor score evaluated on D2, D3, D4, D7, D14, D28, and D56 for subjects in the 600 mg group were 3.5 points (7.00), 3.5 points (7.00), 3.0 points (6.00), 6.0 points (6.93), 10.8 points (9.91), 14.0 points (10.71), and 22.0 points (10.33), respectively.

After receiving the study intervention, the mean change from baseline (standard deviation) in lower limb motor score evaluated on D2, D3, D4, D7, D14, D28, and D56 for subjects in the 1200 mg group were 10.0 points (10.00), 10.0 points (10.00), 10.0 points (10.00), 15.0 points (13.23), 15.0 points (13.23), 15.0 points (13.23), and 15.0 points (13.23), respectively.

After receiving the study intervention, the mean changes (standard deviations) from baseline in lower limb motor score evaluated on D2, D3, D4, D7, D14, D28, and D56 for subjects in the 2400 mg group were 12.7 points (14.19), 18.0 points (8.00), 18.7 points (9.02), 18.0 points (7.21), 23.7 points (6.35), 29.3 points (8.14), and 36.0 points (6.56), respectively.

After receiving the study intervention, the mean changes (standard deviations) from baseline in lower limb motor score evaluated on D2, D3, D4, D7, D14, D28, and D56 for subjects in the 4800 mg group were 2.3 points (4.93), 1.7 points (8.62), 8.0 points (11.36), 9.3 points (12.10), 11.7 points (13.87), 15.0 points (18.73), and 15.3 points (15.63), respectively.

After receiving the study intervention, the mean change from baseline (standard deviation) in lower limb motor score evaluated on D2, D3, D4, D7, D14, D28, and D56 for subjects in the placebo group were 0.3 points (0.82), 3.4 points (5.86), 2.6 points (4.43), 12.9 points (9.77), 16.2 points (10.57), 22.3 points (13.47), and 27.1 points (14.66), respectively.

At the D56 visit, the mean change from baseline in the lower extremity motor score for subjects in the 2400 mg group was higher than that of the placebo group.

TABLE 20
Analysis of Changes from Baseline in Lower Limb Motor Score (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 4 (0) | 3 (0) | 3 (0) | 3 (0) | 7 (0) | 24 (0) |
| | Subjects | | | | | | | |
| | (Missing) | | | | | | | |
| | Mean | 29.3 (16.07) | 5.5 (9.71) | 31.7 (16.07) | 12.3 (9.29) | 8.7 (14.15) | 8.1 (12.17) | 14.8 (15.48) |
| | (Standard | | | | | | | |
| | Deviation) | | | | | | | |
| | Median | 28.0 | 1.0 | 25.0 | 15.0 | 1.0 | 2.0 | 13.0 |
| | Quartiles | 18.5, 40.0 | 0.0, 11.0 | 20.0, 50.0 | 2.0, 20.0 | 0.0, 25.0 | 0.0, 21.0 | 1.5, 25.0 |
| | (Q1, Q3) | | | | | | | |
| | Minimum, | 11, 50 | 0, 20 | 20, 50 | 2, 20 | 0, 25 | 0, 30 | 0, 50 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 2 | (Missing) | | | | | | | |
| | Mean | 3.8 (7.50) | 3.5 (7.00) | 10.0 (10.00) | 12.7 (14.19) | 2.3 (4.93) | 0.3 (0.82) | 4.6 (7.94) |
| | (Standard | | | | | | | |
| | Deviation) | | | | | | | |
| | Median | 0.0 | 0.0 | 10.0 | 10.0 | 0.0 | 0.0 | 0.0 |
| | Quartiles | 0.0, 7.5 | 0.0, 7.0 | 0.0, 20.0 | 0.0, 28.0 | −1.0, 8.0 | 0.0, 0.0 | 0.0, 10.0 |
| | (Q1, Q3) | | | | | | | |
| | Minimum, | 0, 15 | 0, 14 | 0, 20 | 0, 28 | −1, 8 | 0, 2 | −1, 28 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 3 | (Missing) | | | | | | | |
| | Mean | 5.3 (9.22) | 3.5 (7.00) | 10.0 (10.00) | 18.0 (8.00) | 1.7 (8.62) | 3.4 (5.86) | 6.2 (8.58) |
| | (Standard | | | | | | | |
| | Deviation) | | | | | | | |
| | Median | 1.0 | 0.0 | 10.0 | 18.0 | 0.0 | 0.0 | 1.0 |
| | Quartiles | 0.0, 10.5 | 0.0, 7.0 | 0.0, 20.0 | 10.0, 26.0 | −6.0, 11.0 | 0.0, 4.0 | 0.0, 12.5 |
| | (Q1, Q3) | | | | | | | |
| | Minimum, | 0, 19 | 0, 14 | 0, 20 | 10, 26 | −6, 11 | 0, 16 | −6, 26 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 4 | (Missing) | | | | | | | |
| | Mean | 5.0 (11.37) | 3.0 (6.00) | 10.0 (10.00) | 18.7 (9.02) | 8.0 (11.36) | 2.6 (4.43) | 6.7 (9.06) |
| | (Standard | | | | | | | |
| | Deviation) | | | | | | | |
| | Median | 0.0 | 0.0 | 10.0 | 18.0 | 3.0 | 0.0 | 1.0 |
| | Quartiles | −1.0, 11.0 | 0.0, 6.0 | 0.0, 20.0 | 10.0, 28.0 | 0.0, 21.0 | 0.0, 4.0 | 0.0, 12.0 |
| | (Q1, Q3) | | | | | | | |
| | Minimum, | −2, 22 | 0, 12 | 0, 20 | 10, 28 | 0, 21 | 0, 12 | −2, 28 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 7 | (Missing) | | | | | | | |
| | Mean | 9.8 (8.42) | 6.0 (6.93) | 15.0 (13.23) | 18.0 (7.21) | 9.3 (12.10) | 12.9 (9.77) | 11.7 (9.32) |
| | (Standard | | | | | | | |
| | Deviation) | | | | | | | |
| | Median | 10.0 | 4.0 | 20.0 | 20.0 | 5.0 | 10.0 | 10.0 |
| | Quartiles | 3.0, 16.5 | 2.0, 10.0 | 0.0, 25.0 | 10.0, 24.0 | 0.0, 23.0 | 5.0, 22.0 | 4.0, 20.0 |
| | (Q1, Q3) | | | | | | | |
| | Minimum, | 0, 19 | 0, 16 | 0, 25 | 10, 24 | 0, 23 | 0, 28 | 0, 28 |
| | Maximum | | | | | | | |
| Change from | Number of | 4 (0) | 4 (0) | 3 (0) | 3 (0) | 3 (0) | 6 (1) | 23 (1) |
| Baseline at | Subjects | | | | | | | |
| Day 14 | (Missing) | | | | | | | |
| | Mean | 15.0 (11.14) | 10.8 (9.91) | 15.0 (13.23) | 23.7 (6.35) | 11.7 (13.87) | 16.2 (10.57) | 15.3 (10.40) |
| | (Standard | | | | | | | |
| | Deviation) | | | | | | | |
| | Median | 17.0 | 9.5 | 20.0 | 20.0 | 8.0 | 17.0 | 19.0 |
| | Quartiles | 7.0, 23.0 | 4.5, 17.0 | 0.0, 25.0 | 20.0, 31.0 | 0.0, 27.0 | 9.0, 26.0 | 8.0, 25.0 |
| | (Q1, Q3) | | | | | | | |
| | Minimum, | 0, 26 | 0, 24 | 0, 25 | 20, 31 | 0, 27 | 0, 28 | 0, 31 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 28 | (Missing) | | | | | | | |
| | Mean | 14.8 (10.81) | 14.0 (10.71) | 15.0 (13.23) | 29.3 (8.14) | 15.0 (18.73) | 22.3 (13.47) | 18.7 (12.56) |
| | (Standard | | | | | | | |
| | Deviation) | | | | | | | |
| | Median | 17.0 | 15.0 | 20.0 | 33.0 | 9.0 | 26.0 | 20.0 |
| | Quartiles | 7.0, 22.5 | 7.0, 21.0 | 0.0, 25.0 | 20.0, 35.0 | 0.0, 36.0 | 14.0, 36.0 | 11.5, 26.5 |
| | (Q1, Q3) | | | | | | | |
| | Minimum, | 0, 25 | 0, 26 | 0, 25 | 20, 35 | 0, 36 | 0, 38 | 0, 38 |
| | Maximum | | | | | | | |
| Change from | Number of | 4 (0) | 4 (0) | 3 (0) | 3 (0) | 3 (0) | 7 (0) | 24 (0) |
| Baseline at | Subjects | | | | | | | |
| Day 56 | (Missing) | | | | | | | |
| | Mean | 19.5 (14.27) | 22.0 (10.33) | 15.0 (13.23) | 36.0 (6.56) | 15.3 (15.63) | 27.1 (14.66) | 23.1 (13.50) |
| | (Standard | | | | | | | |
| | Deviation) | | | | | | | |
| | Median | 22.0 | 22.0 | 20.0 | 35.0 | 13.0 | 32.0 | 25.5 |
| | Quartiles | 10.0, 29.0 | 14.0, 30.0 | 0.0, 25.0 | 30.0, 43.0 | 1.0, 32.0 | 15.0, 38.0 | 14.0, 34.0 |
| | (Q1, Q3) | | | | | | | |
| | Minimum, | 0, 34 | 10, 34 | 0, 25 | 30, 43 | 1, 32 | 0, 40 | 0, 43 |
| | Maximum | | | | | | | |

Note:
Lower limb motor score (sum of L2-S1 scores); time points were baseline, D 2, D 3, D 4, D 7, D 14, D 28, and D 56.

c. Injury Grading

Based on FAS, all groups of subjects showed an improving trend in injury grading, with the 1200 mg and 2400 mg dose groups exhibiting a better trend compared to the placebo group. For details, see Table 21.

In the 200 mg (or 3 mg/kg) group, the proportions of subjects with injury grades D and E at baseline and at D2, D3, D4, D7, D14, D28, and D56 after receiving the study intervention were 0%, 0%, 25.0%, 25.0%, 50.0%, 75.0%, 100%, and 100%, respectively.

In the 600 mg group, the proportions of subjects with injury grades D and E at baseline and at D2, D3, D4, D7, D14, D28, and D56 after receiving the study intervention were 0%, 0%, 0%, 0%, 0%, 50.0%, 50.0%, and 50.0%, respectively.

In the 1200 mg group, the proportions of subjects with injury grades D and E at baseline and at D2, D3, D4, D7, D14, D28, and D56 after receiving the study intervention were 0%, 66.7%, 66.7%, 66.7%, 100%, 100%, 100%, and 100%, respectively.

In the 2400 mg group, the proportions of subjects with injury grades D and E at baseline and at D2, D3, D4, D7, D14, D28, and D56 after receiving the study intervention were 0%, 66.7%, 66.7%, 66.7%, 66.7%, 100%, 100%, and 100%, respectively.

In the 4800 mg group, the proportions of subjects with injury grades D and E at baseline and at D2, D3, D4, D7, D14, D28, and D56 after receiving the study intervention were 0%, 0%, 0%, 0%, 33.3%, 33.3%, 66.7%, and 66.7%, respectively.

In the placebo group, the proportions of subjects with injury grades D and E at baseline and at D2, D3, D4, D7, D14, D28, and D56 after receiving the study intervention were 0%, 0%, 0%, 0%, 28.6%, 42.9%, 57.1%, and 85.7%, respectively.

TABLE 21
Injury Grading Analysis (FAS).

| | | 3 mg/kg | | | | | Placebo | |
|---|---|---|---|---|---|---|---|---|
| | ASIA | or 200 mg | 600 mg | 1200 mg | 2400 mg | 4800 mg | Group | Total |
| | Impairment | (N = 4) | (N = 4) | (N = 3) | (N = 3) | (N = 3) | (N = 7) | (N = 24) |
| Visits | Scale | n (%) | n (%) | n (%) | n (%) | n (%) | n (%) | n (%) |
| Baseline | | | | | | | | |
| | A | 0 | 0 | 0 | 0 | 0 | 0 | 0 |
| | B | 0 | 2 (50.0) | 0 | 0 | 1 (33.3) | 1 (14.3) | 4 (16.7) |
| | C | 4 (100) | 2 (50.0) | 3 (100) | 3 (100) | 2 (66.7) | 6 (85.7) | 20 (83.3) |
| | D | 0 | 0 | 0 | 0 | 0 | 0 | 0 |
| | E | 0 | 0 | 0 | 0 | 0 | 0 | 0 |
| D 2 | | | | | | | | |
| | A | 0 | 0 | 0 | 0 | 0 | 0 | 0 |
| | B | 0 | 2 (50.0) | 0 | 0 | 1 (33.3) | 0 | 3 (12.5) |
| | C | 4 (100) | 2 (50.0) | 1 (33.3) | 1 (33.3) | 2 (66.7) | 6 (85.7)* | 16 (66.7) |
| | D | 0 | 0 | 2 (66.7) | 2 (66.7) | 0 | 0 | 4 (16.7) |
| | E | 0 | 0 | 0 | 0 | 0 | 0 | 0 |
| D 3 | | | | | | | | |
| | A | 0 | 0 | 0 | 0 | 0 | 0 | 0 |
| | B | 0 | 2 (50.0) | 0 | 0 | 1 (33.3) | 0 | 3 (12.5) |
| | C | 3 (75.0) | 2 (50.0) | 1 (33.3) | 1 (33.3) | 2 (66.7) | 7 (100) | 16 (66.7) |
| | D | 1 (25.0) | 0 | 2 (66.7) | 2 (66.7) | 0 | 0 | 5 (20.8) |
| | E | 0 | 0 | 0 | 0 | 0 | 0 | 0 |
| D 4 | | | | | | | | |
| | A | 0 | 0 | 0 | 0 | 0 | 0 | 0 |
| | B | 0 | 2 (50.0) | 0 | 0 | 1 (33.3) | 0 | 3 (12.5) |
| | C | 3 (75.0) | 2 (50.0) | 1 (33.3) | 1 (33.3) | 2 (66.7) | 7 (100) | 16 (66.7) |
| | D | 1 (25.0) | 0 | 2 (66.7) | 2 (66.7) | 0 | 0 | 5 (20.8) |
| | E | 0 | 0 | 0 | 0 | 0 | 0 | 0 |
| D 7 | | | | | | | | |
| | A | 0 | 0 | 0 | 0 | 0 | 0 | 0 |
| | B | 0 | 1 (25.0) | 0 | 0 | 1 (33.3) | 0 | 2 (8.3) |
| | C | 2 (50.0) | 3 (75.0) | 0 | 1 (33.3) | 1 (33.3) | 5 (71.4) | 12 (50.0) |
| | D | 2 (50.0) | 0 | 3 (100) | 2 (66.7) | 1 (33.3) | 2 (28.6) | 10 (41.7) |
| | E | 0 | 0 | 0 | 0 | 0 | 0 | 0 |
| D 14 | | | | | | | | |
| | A | 0 | 0 | 0 | 0 | 0 | 0 | 0 |
| | B | 0 | 1 (25.0) | 0 | 0 | 0 | 0 | 1 (4.2) |
| | C | 1 (25.0) | 1 (25.0) | 0 | 0 | 2 (66.7) | 3 (42.9)* | 7 (29.2) |
| | D | 3 (75.0) | 2 (50.0) | 3 (100) | 3 (100) | 1 (33.3) | 3 (42.9)* | 15 (62.5) |
| | E | 0 | 0 | 0 | 0 | 0 | 0 | 0 |
| D 28 | | | | | | | | |
| | A | 0 | 0 | 0 | 0 | 0 | 0 | 0 |
| | B | 0 | 1 (25.0) | 0 | 0 | 0 | 0 | 1 (4.2) |
| | C | 0 | 1 (25.0) | 0 | 0 | 1 (33.3) | 3 (42.9) | 5 (20.8) |
| | D | 4 (100) | 2 (50.0) | 3 (100) | 3 (100) | 2 (66.7) | 4 (57.1) | 18 (75.0) |
| | E | 0 | 0 | 0 | 0 | 0 | 0 | 0 |
| D 56 | | | | | | | | |
| | A | 0 | 0 | 0 | 0 | 0 | 0 | 0 |
| | B | 0 | 0 | 0 | 0 | 0 | 0 | 0 |
| | C | 0 | 2 (50.0) | 0 | 0 | 1 (33.3) | 1 (14.3) | 4 (16.7) |
| | D | 3 (75.0) | 2 (50.0) | 2 (66.7) | 3 (100) | 2 (66.7) | 6 (85.7) | 18 (75.0) |
| | E | 1 (25.0) | 0 | 1 (33.3) | 0 | 0 | 0 | 2 (8.3) |

Abbreviation: ASIA = American Spinal Injury Association.
Note:
Time points are baseline, D 2, D 3, D 4, D 7, D 14, D 28, and D 56.
*One subject in the placebo group was unable to attend or refused the visit at both the D 2 and D 14 visits, resulting in missing data.

d. Pain Visual Analogue Scale

Based on FAS, the analysis of pain Visual Analog Scale scores and their changes from baseline are shown in Table 22.

After receiving the study intervention, the mean change from baseline (standard deviation) in pain visual analog scale scores on D2, D3, D4, D7, D14, D28, and D56 for subjects in the 200 mg (or 3 mg/kg) group were 1.5 points (13.18), 11.3 points (24.23), 12.3 points (26.21), −0.3 points (30.39), 5.5 points (48.88), −19.3 points (26.25), and −15.8 points (46.89), respectively.

After receiving the study intervention, the mean change from baseline (standard deviation) in pain visual analog scale scores on D2, D3, D4, D7, D14, D28, and D56 for subjects in the 600 mg group were 6.7 points (20.82), 6.3 points (20.98), −14.3 points (14.01), −13.0 points (15.72), −2.3 points (15.70), −6.7 points (20.82), and −9.0 points (27.07), respectively.

After receiving the study intervention, the mean change from baseline (standard deviation) in pain visual analog scale scores on D2, D3, D4, D7, D14, D28, and D56 for subjects in the 1200 mg group were 3.0 points (15.39), −2.7 points (20.53), −9.3 points (16.77), −10.0 points (26.46), −29.0 points (16.52), −40.0 points (22.91), and −44.3 points (27.68), respectively.

After receiving the study intervention, the mean change from baseline (standard deviation) in pain visual analog scale scores on D2, D3, D4, D7, D14, D28, and D56 for subjects in the 2400 mg group were 10.0 points (20.88), −1.0 point (8.54), −3.0 points (13.75), −16.7 points (28.87), −15.3 points (26.56), −23.7 points (40.99), and −23.7 points (40.99), respectively.

After receiving the study intervention, the mean change from baseline (standard deviation) in pain visual analog scale scores on D2, D3, D4, D7, D14, D28, and D56 for subjects in the 4800 mg group were −7.3 points (4.51), −12.3 points (9.240), −9.0 points (7.00), −12.3 points (11.37), −13.7 points (10.50), −16.3 points (9.07), and −12.3 points (7.37), respectively.

After receiving the study intervention, the mean change from baseline (standard deviation) in pain visual analog scale scores on D2, D3, D4, D7, D14, D28, and D56 for subjects in the placebo group were 2.7 points (35.39), −7.0 points (37.98), 11.3 points (17.59), −10.7 points (41.94), −15.7 points (34.88), −18.9 points (36.18), and −20.9 points (35.61), respectively.

At the D56 visit, the mean change from baseline in pain visual analog scale scores for subjects in the 1200 mg and 2400 mg group was lower than that of the placebo group.

TABLE 22
Analysis of Changes from Baseline in Pain Visual Analog Scale Score (FAS).

| | | 3 mg/kg | | | | | Placebo | |
|---|---|---|---|---|---|---|---|---|
| | | or 200 mg | 600 mg | 1200 mg | 2400 mg | 4800 mg | Group | Total |
| Visits | | (N = 4) | (N = 4) | (N = 3) | (N = 3) | (N = 3) | (N = 7) | (N = 24) |
| Baseline | Number of | 4 (0) | 3 (1) | 3 (0) | 3 (0) | 3 (0) | 7 (0) | 23 (1) |
| | Subjects | | | | | | | |
| | (Missing) | | | | | | | |
| | Mean | 37.8 (29.10) | 38.0 (29.87) | 46.3 (25.11) | 23.7 (40.99) | 17.3 (8.50) | 35.9 (38.87) | 33.8 (30.07) |
| | (Standard | | | | | | | |
| | Deviation) | | | | | | | |
| | Median | 40.5 | 50.0 | 49.0 | 0.0 | 17.0 | 21.0 | 26.0 |
| | Quartiles | 17.5, 58.0 | 4.0, 60.0 | 20.0, 70.0 | 0.0, 71.0 | 9.0, 26.0 | 3.0, 82.0 | 3.0, 60.0 |
| | (Q1, Q3) | | | | | | | |
| | Minimum, | 0, 70 | 4, 60 | 20, 70 | 0, 71 | 9, 26 | 0, 91 | 0, 91 |
| | Maximum | | | | | | | |
| Change from | Number of | 4 (0) | 3 (1) | 3 (0) | 3 (0) | 3 (0) | 6 (1) | 22 (2) |
| Baseline at | Subjects | | | | | | | |
| Day 2 | (Missing) | | | | | | | |
| | Mean | 1.5 (13.18) | 6.7 (20.82) | 3.0 (15.39) | 10.0 (20.88) | −7.3 (4.51) | 2.7 (35.39) | 2.7 (21.33) |
| | (Standard | | | | | | | |
| | Deviation) | | | | | | | |
| | Median | 3.5 | 0.0 | −1.0 | 0.0 | −7.0 | 5.0 | 0.0 |
| | Quartiles | −8.0, 11.0 | −10.0, 30.0 | −10.0, 20.0 | −4.0, 34.0 | −12.0, −3.0 | −18.0, 26.0 | −10.0, 15.0 |
| | (Q1, Q3) | | | | | | | |
| | Minimum, | −16, 15 | −10, 30 | −10, 20 | −4, 34 | −12, −3 | −52, 50 | −52, 50 |
| | Maximum | | | | | | | |
| Change from | Number of | 4 (0) | 3 (1) | 3 (0) | 3 (0) | 3 (0) | 7 (0) | 23 (1) |
| Baseline at | Subjects | | | | | | | |
| Day 3 | (Missing) | | | | | | | |
| | Mean | 11.3 (24.23) | 6.3 (20.98) | −2.7 (20.53) | −1.0 (8.54) | −12.3 (9.24) | −7.0 (37.98) | −1.4 (25.10) |
| | (Standard | | | | | | | |
| | Deviation) | | | | | | | |
| | Median | 5.5 | −1.0 | −8.0 | 0.0 | −7.0 | 2.0 | −1.0 |
| | Quartiles | −5.5, 28.0 | −10.0, 30.0 | −20.0, 20.0 | −10.0, 7.0 | −23.0, −7.0 | −41.0, 6.0 | −10.0, 7.0 |
| | (Q1, Q3) | | | | | | | |
| | Minimum, | −11, 45 | −10, 30 | −20, 20 | −10, 7 | −23, −7 | −69, 50 | −69, 50 |
| | Maximum | | | | | | | |
| Change from | Number of | 4 (0) | 3 (1) | 3 (0) | 3 (0) | 3 (0) | 7 (0) | 23 (1) |
| Baseline at | Subjects | | | | | | | |
| Day 4 | (Missing) | | | | | | | |
| | Mean | 12.3 (26.21) | −14.3 (14.01) | −9.3 (16.77) | −3.0 (13.75) | −9.0 (7.00) | 11.3 (17.59) | 0.9 (19.03) |
| | (Standard | | | | | | | |
| | Deviation) | | | | | | | |
| | Median | 8.0 | −10.0 | −18.0 | 0.0 | −9.0 | 7.0 | 0.0 |
| | Quartiles | −7.0, 31.5 | −30.0, −3.0 | −20.0, 10.0 | −18.0, 9.0 | −16.0, −2.0 | 0.0, 10.0 | −14.0, 9.0 |
| | (Q1, Q3) | | | | | | | |
| | Minimum, | −14, 47 | −30, −3 | −20, 10 | −18, 9 | −16, −2 | −1, 50 | −30, 50 |
| | Maximum | | | | | | | |
| Change from | Number of | 4 (0) | 3 (1) | 3 (0) | 3 (0) | 3 (0) | 7 (0) | 23 (1) |
| Baseline at | Subjects | | | | | | | |
| Day 7 | (Missing) | | | | | | | |
| | Mean | −0.3 (30.39) | −13.0 (15.72) | −10.0 (26.46) | −16.7 (28.87) | −12.3 (11.37) | −10.7 (41.94) | −10.1 (28.37) |
| | (Standard | | | | | | | |
| | Deviation) | | | | | | | |
| | Median | −2.5 | −10.0 | −20.0 | 0.0 | −9.0 | −11.0 | −9.0 |
| | Quartiles | −20.0, 19.5 | −30.0, 1.0 | −30.0, 20.0 | −50.0, 0.0 | −25.0, −3.0 | −19.0, 6.0 | −25.0, 1.0 |
| | (Q1, Q3) | | | | | | | |
| | Minimum, | −35, 39 | −30, 1 | −30, 20 | −50, 0 | −25, −3 | −90, 50 | −90, 50 |
| | Maximum | | | | | | | |
| Change from | Number of | 4 (0) | 3 (1) | 3 (0) | 3 (0) | 3 (0) | 6 (1) | 22 (2) |
| Baseline at | Subjects | | | | | | | |
| Day 14 | (Missing) | | | | | | | |
| | Mean | 5.5 (48.88) | −2.3 (15.70) | −29.0 (16.52) | −15.3 (26.56) | −13.7 (10.50) | −15.7 (34.88) | −11.5 (29.61) |
| | (Standard | | | | | | | |
| | Deviation) | | | | | | | |
| | Median | −0.5 | 3.0 | −37.0 | 0.0 | −14.0 | 0.0 | −6.5 |
| | Quartiles | −34.0, 45.0 | −20.0, 10.0 | −40.0, −10.0 | −46.0, 0.0 | −24.0, −3.0 | −17.0, 2.0 | −26.0, 2.0 |
| | (Q1, Q3) | | | | | | | |
| | Minimum, | −42, 65 | −20, 10 | −40, −10 | −46, 0 | −24, −3 | −85, 6 | −85, 65 |
| | Maximum | | | | | | | |
| Change from | Number of | 4 (0) | 3 (1) | 3 (0) | 3 (0) | 3 (0) | 7 (0) | 23 (1) |
| Baseline at | Subjects | | | | | | | |
| Day 28 | (Missing) | | | | | | | |
| | Mean | −19.3 (26.25) | −6.7 (20.82) | −40.0 (22.91) | −23.7 (40.99) | −16.3 (9.07) | −18.9 (36.18) | −20.4 (27.95) |
| | (Standard | | | | | | | |
| | Deviation) | | | | | | | |
| | Median | −16.0 | 0.0 | −45.0 | 0.0 | −15.0 | −2.0 | −15.0 |
| | Quartiles | −41.0, 2.5 | −30.0, 10.0 | −60.0, −15.0 | − 71.0, 0.0 | −26.0, −8.0 | −33.0, 2.0 | −33.0, 0.0 |
| | (Q1, Q3) | | | | | | | |
| | Minimum, | −50, 5 | −30, 10 | −60, −15 | −71, 0 | −26, −8 | −90, 19 | −90, 19 |
| | Maximum | | | | | | | |
| Change from | Number of | 4 (0) | 3 (1) | 3 (0) | 3 (0) | 3 (0) | 7 (0) | 23 (1) |
| Baseline at | Subjects | | | | | | | |
| Day 56 | (Missing) | | | | | | | |
| | Mean | −15.8 (46.89) | −9.0 (27.07) | −44.3 (27.68) | −23.7 (40.99) | −12.3 (7.37) | −20.9 (35.61) | −20.7 (32.40) |
| | (Standard | | | | | | | |
| | Deviation) | | | | | | | |
| | Median | −16.5 | 3.0 | −48.0 | 0.0 | −15.0 | −3.0 | −15.0 |
| | Quartiles | −51.5, 20.0 | −40.0, 10.0 | −70.0, −15.0 | − 71.0, 0.0 | −18.0, −4.0 | −38.0, 0.0 | −40.0, 0.0 |
| | (Q1, Q3) | | | | | | | |
| | Minimum, | −70, 40 | −40, 10 | −70, −15 | −71, 0 | −18, −4 | −89, 16 | −89, 40 |
| | Maximum | | | | | | | |

Note:
Time points were screening, Day 2, Day 3, Day 4, Day 7, Day 14, Day 28, and Day 56.

### 2. Efficacy Summary

At day 56, patients treated with ALMB-0166 had improvement as compared with those given placebo in motor function (scores increased from baseline by 66.0 for 2400 mg and 45.6 for placebo), sensatory function (scores increased by 77.5, 62.7 for 600, 1200 mg and 52.3 for placebo), AIS (2 patients recovered from grade C to grade E for ALMB-0166 and 0 patient recovered to grade E for placebo), and pain (VAS scores decreased by 44.3, 23.7 for 1200, 2400 mg and 20.9 for placebo).

a. ASIA Sensory Score

At the D56 visit, the mean change from baseline in the ASIA sensory total score for subjects in the 600 mg and 1200 mg groups was higher than that of the placebo group.

At the D56 visit, the mean change from baseline in the left ASIA total sensory score for subjects in the 600 mg group was higher than that of the placebo group.

At the D56 visit, the mean change from baseline in ASIA sensory total score—right score for subjects in the 200 mg (or 3 mg/kg) group, 600 mg group, 1200 mg, and 2400 mg groups was higher than that of the placebo group.

At the D56 visit, the mean changes from baseline in total pinprick sensation scores for subjects in the 200 mg (or 3 mg/kg) group, 600 mg group, and 1200 mg group were higher than those in the placebo group.

At the D56 visit, the mean change from baseline in total light touch scores for subjects in the 600 mg and 1200 mg groups was higher than that of the placebo group.

b. Motor Score

At the D56 visit, the mean change from baseline in the total motor score for subjects in the 2400 mg group was higher than that of the placebo group.

At the D56 visit, the mean change from baseline in the total motor score—left side score for subjects in the 2400 mg group was higher than that of the placebo group.

At the D56 visit, the mean change from baseline in the Total Motor Score−right side score for subjects in the 200 mg (or 3 mg/kg) group and the 2400 mg group was higher than that of the placebo group.

At the D56 visit, the mean change from baseline in upper limb motor scores for subjects in the 200 mg (or 3 mg/kg) group, the 1200 mg group, and the 2400 mg group was higher than that of the placebo group.

At the D56 visit, the mean change from baseline in the lower extremity motor score for subjects in the 2400 mg group was higher than that of the placebo group.

### 3. Pharmacokinetic Results

a. Analysis of Plasma Concentrations

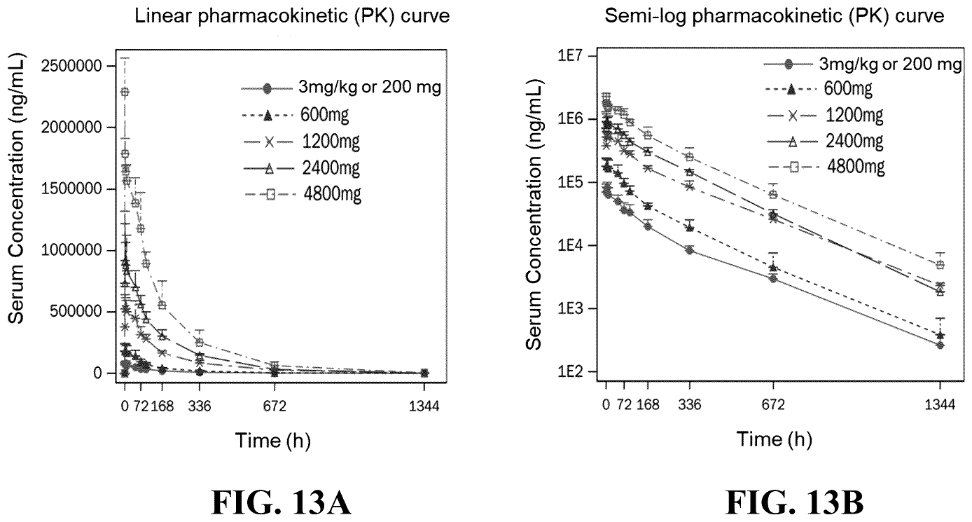

After administration of different doses of ALMB-0166 (200 mg [or 3 mg/kg], 600 mg, 1200 mg, 2400 mg, and 4800 mg), the process of ALMB-0166 serum concentration over time was similar across dose groups. The concentration gradually increased from the start of infusion, basically peaked at the end of infusion, and then decreased slowly. The mean ALMB-0166 time-concentration curves for each group are shown in FIGS. 13A-13B.

b. PK Parameter Analysis

Following a single intravenous infusion of ALMB-0166 at doses of 200 mg (or 3 mg/kg) to 4800 mg in subjects, the median Tₘₐₓ of ALMB-0166 in serum for each dose group was 1.03-2.01 h. The in vivo exposure of ALMB-0166 increased with the administered dose; the arithmetic mean Cₘₐₓ ranged from 76.80 to 2290.00 g/mL, and the arithmetic mean AUC₀₋ₜ and AUC_{0-∞} were 9.80 to 256.06 h mg/mL and 9.88 to 257.29 h mg/mL, respectively. There were no significant differences among dose groups in the elimination half-life, volume of distribution, and clearance of ALMB-0166; the arithmetic mean t_{1/2} ranged from 149.86 to 201.24 hours, the arithmetic mean V ranged from 4.00 to 6.34 L, and the arithmetic mean CL ranged from 14.29 to 30.94 mL/h. The PK parameter analysis of ALMB-0166 in serum after a single intravenous infusion in each dose group is shown in Table 23.

TABLE 23
Summary of ALMB-0166 PK Parameters (PKPS).

| Dose | | | | | | | λ_{z} | | | |
|---|---|---|---|---|---|---|---|---|---|---|
| Groups | | Tₘₐₓ | Cₘₐₓ | AUC₀₋ₜ | AUC_{0-∞} | % AUCₑₓ | (10⁻³ × | t_{1/2} | V | CL |
| (mg) | Statistic | (h) | (ug/mL) | (h*mg/mL) | (h*mg/mL) | (%) | 1/h) | (h) | (L) | (mL/h) |
| 3 mg/kg or | n | 4 | 4 | 4 | 4 | 4 | 4 | 4 | 4 | 4 |
| 200 mg | | | | | | | | | | |
| (N = 4) | | | | | | | | | | |
| | Mean | 1.60 | 76.80 | 9.80 | 9.88 | 0.78 | 3.45 | 201.24 | 6.22 | 21.38 |
| | % CV | 1.03-4.98 | 18.2 | 27.3 | 27.4 | 30.2 | 3.6 | 3.5 | 28.2 | 26.5 |
| 600 mg | n | 4 | 4 | 4 | 4 | 4 | 4 | 4 | 4 | 4 |
| (N = 4) | | | | | | | | | | |
| | Mean | 2.01 | 181.75 | 19.57 | 21.01 | 9.24 | 5.33 | 151.66 | 6.34 | 30.94 |
| | % CV | 0.983-2.07 | 34.2 | 41.9 | 32.0 | 173.1 | 52.2 | 37.4 | 34.3 | 32.3 |
| 1200 mg | n | 3 | 3 | 3 | 3 | 3 | 3 | 3 | 3 | 3 |
| (N = 3) | | | | | | | | | | |
| | Mean | 1.03 | 575.67 | 84.44 | 85.09 | 0.77 | 3.59 | 193.27 | 4.00 | 14.29 |
| | % CV | 1.02-9.02 | 15.2 | 13.6 | 13.6 | 9.8 | 4.7 | 4.6 | 19.1 | 14.6 |
| 2400 mg | n | 3 | 3 | 3 | 3 | 3 | 3 | 3 | 3 | 3 |
| (N = 3) | | | | | | | | | | |
| | Mean | 1.08 | 946.33 | 108.57 | 126.81 | 14.82 | 5.20 | 149.86 | 4.10 | 19.20 |
| | % CV | 1.02-1.95 | 24.6 | 30.2 | 15.1 | 135.3 | 45.6 | 36.2 | 35.7 | 14.0 |
| 4800 mg | n | 3 | 3 | 3 | 3 | 3 | 3 | 3 | 3 | 3 |
| (N = 3) | | | | | | | | | | |
| | Mean | 1.07 | 2290.00 | 256.06 | 257.29 | 0.45 | 3.96 | 175.92 | 4.84 | 19.32 |
| | % CV | 1.03-1.10 | 12.1 | 21.2 | 21.3 | 49.4 | 8.3 | 8.1 | 16.2 | 24.3 |

Abbreviations: AUC₀₋ₜ = Area under the concentration-time curve from time 0 to t; AUC_{0-∞} = Area under the concentration-time curve extrapolated from time 0 to infinity; CL = clearance; Cₘₐₓ = maximum concentration; CV = coefficient of variation; Tₘₐₓ = time to reach maximum concentration; t_{1/2} = elimination half-life; V = volume of distribution; λ_{z} = terminal rate constant; % AUCₑₓ = percentage of total AUC extrapolated.
Note:
Tₘₐₓ is presented as median and min-max, while other PK parameters are presented as mean and CV %.
Source: Statistical Table 14.4.1.2

c. Dose Proportionality Analysis

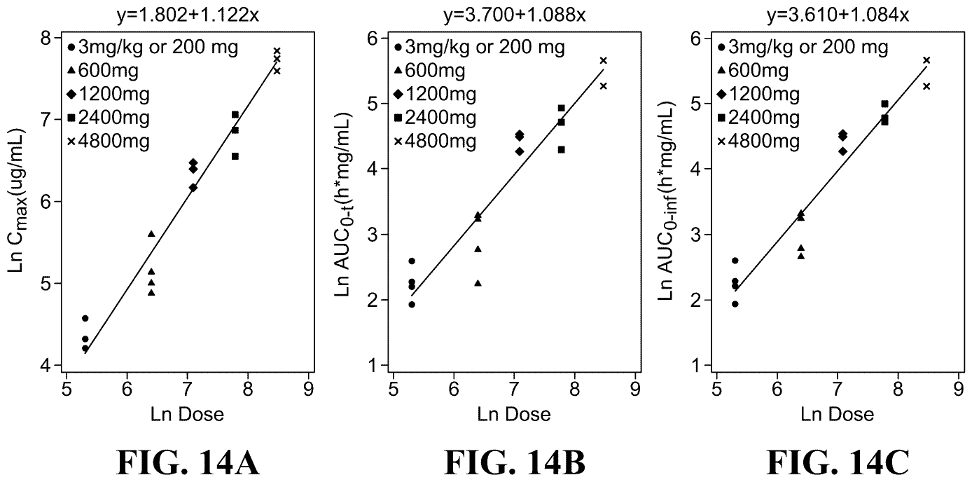

In the dose-escalation range of this study (200 mg [or 3 mg/kg] to 4800 mg), after a single intravenous drip infusion, the R values and their 95% CIs for Cₘₐₓ, AUC0-t, and AUC0-∞ of ALMB-0166 were 1.122 (1.020-1.224), 1.088 (0.929-1.246), and 1.084 (0.956-1.212), respectively; the R values were all close to 1, and their 95% CIs included or were close to 1, indicating that ALMB-0166 exhibited basically linear PK characteristics within the dose range of 200 mg (or 3 mg/kg) to 4800 mg in vivo. Changes in ALMB-0166 PK parameters with dose are shown in Table 24 and FIGS. 14A-14C.

TABLE 24
Relationship between Cmax, AUC0-last,
AUC0-∞, and Dose of ALMB-0166 (PKPS).

| Parameter | | | | Estimated | Power | |
|---|---|---|---|---|---|---|
| (Unit) | Dose Range | n | | Value | Model SE | 90% CI |
| Cₘₐₓ | 200 mg-4800 mg | 17 | β0 | −1.802 | 0.437 | −2.568~−1.036 |
| (ug/mL) | | | β1 | 1.122 | 0.058 | 1.020~1.224 |
| AUC₀₋ₗₐₛₜ | 200 mg-4800 mg | 17 | β0 | −3.700 | 0.674 | −4.882~−2.518 |
| (h*mg/mL) | | | β1 | 1.088 | 0.090 | 0.929 ~1.246 |
| AUC_{0-∞} | 200 mg-4800 mg | 17 | β0 | −3.610 | 0.539 | −4.555~−2.665 |
| (h*mg/mL) | | | β1 | 1.084 | 0.073 | 0.956~1.212 |

Abbreviations: AUC₀₋ₗₐₛₜ = Area under the plasma concentration-time curve from 0 to the last measurable concentration; AUC_{0-∞} = Area under the plasma concentration-time curve from 0 extrapolated to infinity; CI = Confidence Interval; Cₘₐₓ = Maximum concentration; SE = Standard Error.

### 4. Pharmacokinetic Summary

Following a single intravenous infusion of ALMB-0166 at doses of 200 mg (or 3 mg/kg) to 4800 mg in subjects, the median Tₘₐₓ of ALMB-0166 in serum for each dose group was 1.03-2.01 h, the arithmetic mean V was 4.00-6.34 L. Subsequently, the plasma drug concentration decreased slowly, with the arithmetic mean of t_{1/2} being 149.86-201.24 h, and the arithmetic mean of CL being 14.29-30.94 mL/h. The systemic exposure of ALMB-0166 increased as the administered dose increased. Within the dosage range of 200 mg (or 3 mg/kg) to 4800 mg, ALMB-0166 exhibited approximately linear PK characteristics.

### IV. SAFETY EVALUATION

### A. Extent of Exposure

Based on the SS, the drug exposure in each group is shown in Table 25.

In 4 subjects of the 200 mg (or 3 mg/kg) group, the median total dose of drug exposure was 202.5 mg (range: 200, 243), and the median total infusion time was 61.0 min (range: 60, 72).

In 4 subjects of the 600 mg group, the median total dose of drug exposure was 600.0 mg (range: 600, 600), and the median total infusion time was 59.0 min (range: 58, 116).

In 3 subjects of the 1200 mg group, the median total dose of drug exposure was 1200.0 mg (range: 1200, 1200), and the median total infusion time was 59.0 min (range: 59, 59).

In 3 subjects of the 2400 mg group, the median total dose of drug exposure was 2400.0 mg (range: 2400, 2400), and the median total infusion time was 57.0 min (range: 57, 60).

In 3 subjects of the 4800 mg group, the median total dose of drug exposure was 4800.0 mg (range: 4800, 4800), and the median total infusion time was 59.0 min (range: 55, 62).

In 7 subjects of the placebo group, the median total dose of drug exposure was 600.0 mg (range: 195, 4800), and the median total infusion time was 60.0 min (range: 56, 60).

TABLE 25
Drug Exposure (SS).

| | | | | | | |
|---|---|---|---|---|---|---|
| 3 mg/kg or | | | | | Placebo | |
| 200 mg | 600 mg | 1200 mg | 2400 mg | 4800 mg | Group | Total |
| (N = 4) | (N = 4) | (N = 3) | (N = 3) | (N = 3) | (N = 7) | (N = 24) |

Total Drug Exposure (mg)

| | | | | | | | |
|---|---|---|---|---|---|---|---|
| Number of | 4 | 4 | 3 | 3 | 3 | 7 | 24 |
| Subjects (Missing) | | | | | | | |
| Mean (Standard | 212.0 (20.74) | 600.0 (0.00) | 1200.0 (0.00) | 2400.0 (0.00) | 4800.0 (0.00) | 1432.7 (1667.54) | 1603.2 (1637.96) |
| Deviation) | | | | | | | |
| Median | 202.5 | 600.0 | 1200.0 | 2400.0 | 4800.0 | 600.0 | 900.0 |
| Quartiles (Q1, | 200.5, 223.5 | 600.0, 600.0 | 1200.0, 1200.0 | 2400.0, 2400.0 | 4800.0, 4800.0 | 234.0, 2400.0 | 421.5, 2400.0 |
| Q3) | | | | | | | |
| Minimum, | 200, 243 | 600, 600 | 1200, 1200 | 2400, 2400 | 4800, 4800 | 195, 4800 | 195, 4800 |
| Maximum | | | | | | | |

Total drug infusion time (min)

| | | | | | | | |
|---|---|---|---|---|---|---|---|
| Number of | 4 | 4 | 3 | 3 | 3 | 7 | 24 |
| Subjects (Missing) | | | | | | | |
| Mean (Standard | 63.5 (5.69) | 73.0 (28.68) | 59.0 (0.00) | 58.0 (1.73) | 58.7 (3.51) | 58.4 (1.99) | 61.8 (11.99) |
| Deviation) | | | | | | | |
| Median | 61.0 | 59.0 | 59.0 | 57.0 | 59.0 | 60.0 | 59.5 |
| Quartiles (Q1, | 60.5, 66.5 | 58.0, 88.0 | 59.0, 59.0 | 57.0, 60.0 | 55.0, 62.0 | 56.0, 60.0 | 57.5, 60.0 |
| Q3) | | | | | | | |
| Minimum, | 60, 72 | 58, 116 | 59, 59 | 57, 60 | 55, 62 | 56, 60 | 55, 116 |
| Maximum | | | | | | | |

Note:
Percentages were calculated based on the number of subjects in the SS for each group.

### B. Adverse Events

### 1. Brief Summary of Adverse Events

A summary of TEAEs is provided in Table 26. Based on the SS, 16 subjects (94.1%) in the ALMB-0166 group (N=17) experienced at least 1 TEAE, and all 7 subjects (100%) in the placebo group (N=7) experienced at least 1 TEAE. In the ALMB-0166 group, 6 subjects (35.3%) experienced at least 1 TEAE related to the study drug, and in the placebo group, 2 subjects (28.6%) experienced at least 1 TEAE related to the study drug. The incidence of TEAEs and TEAEs related to the study drug was similar between the two groups.

The proportion of TEAEs that occurred in each ALMB-0166 group (200 mg [or 3 mg/kg] group, 600 mg group, 1200 mg group, 2400 mg group, and 4800 mg) was 100%, 100%, 100%, 66.7%, and 100%, respectively. The incidence of TEAEs related to the study drug in each ALMB-0166 group was 50.0%, 0, 33.3%, 66.7%, and 33.3%, respectively.

No subjects experienced TEAEs leading to death; only 1 subject (5.9%) in the ALMB-0166 group experienced an SAE, which was judged as unlikely related to the study drug. No subjects experienced TEAEs leading to permanent discontinuation or temporary interruption of the study drug.

TABLE 26
Summary of TEAEs During Treatment (SS).

| | | | | | | ALMB- | | |
|---|---|---|---|---|---|---|---|---|
| | 3 mg/kg | | | | | 0166 | Placebo | |
| | or 200 mg | 600 mg | 1200 mg | 2400 mg | 4800 mg | Group | Group | Total |
| | (N = 4) | (N = 4) | (N = 3) | (N = 3) | (N = 3) | (N = 17) | (N = 7) | (N = 24) |
| | n (%) | n (%) | n (%) | n (%) | n (%) | n (%) | n (%) | n (%) |
| Subjects with at | 4 (100) | 4 (100) | 3 (100) | 2 (66.7) | 3 (100) | 16 (94.1) | 7 (100) | 23 (95.8) |
| least one TEAE | | | | | | | | |
| Grade ≥3 TEAEs | 0 | 2 (50.0) | 0 | 0 | 1 (33.3) | 3 (17.6) | 3 (42.9) | 6 (25.0) |
| SAEs | 0 | 1 (25.0) | 0 | 0 | 0 | 1 (5.9) | 0 | 1 (4.2) |
| TEAEs leading to | 0 | 0 | 0 | 0 | 0 | 0 | 0 | 0 |
| death | | | | | | | | |
| TEAEs leading to | 0 | 0 | 0 | 0 | 0 | 0 | 0 | 0 |
| permanent | | | | | | | | |
| discontinuation | | | | | | | | |
| TEAEs leading to | 0 | 0 | 0 | 0 | 0 | 0 | 0 | 0 |
| dose interruption | | | | | | | | |
| TEAEs related to | 2 (50.0) | 0 | 1 (33.3) | 2 (66.7) | 1 (33.3) | 6 (35.3) | 2 (28.6) | 8 (33.3) |
| study drug | | | | | | | | |
| Grade ≥3 TEAEs | 0 | 0 | 0 | 0 | 0 | 0 | 0 | 0 |
| SAEs | 0 | 0 | 0 | 0 | 0 | 0 | 0 | 0 |
| TEAEs leading to | 0 | 0 | 0 | 0 | 0 | 0 | 0 | 0 |
| death | | | | | | | | |
| TEAEs leading to | 0 | 0 | 0 | 0 | 0 | 0 | 0 | 0 |
| permanent | | | | | | | | |
| discontinuation | | | | | | | | |
| TEAEs leading to | 0 | 0 | 0 | 0 | 0 | 0 | 0 | 0 |
| dose interruption | | | | | | | | |

Abbreviation: TEAE = Treatment-emergent adverse event.
Note:
TEAE: Events that occurred during treatment that did not appear before treatment or worsened compared to before treatment; percentages were calculated based on the number of subjects in the SS for each group; related to the study drug: causal relationship with the study drug is definitely related, probably related, or possibly related.

### 2. Display of Adverse Events

TEAEs by SOC and PT are shown in Table 27.

Based on the SS, in the ALMB-0166 group (N=17), 16 subjects (94.1%) experienced at least 1 TEAE. By PT, TEAEs with an incidence of ≥10% included hyponatraemia in 6 subjects (35.3%); hypokalaemia, constipation, pyrexia, and anaemia in 5 subjects (29.4%) each; white blood cell count increased, alanine aminotransferase increased, aspartate aminotransferase increased, red blood cells urine positive, and fibrin D-dimer increased in 3 subjects (17.6%) each; hypoalbuminaemia, C-reactive protein increased, urinary occult blood positive, glucose urine present, diarrhoea, sinus bradycardia, urinary tract infection, procedural pain, and deep vein thrombosis in 2 subjects each (11.8%).

In the placebo group (N=7), all 7 subjects (100%) experienced at least 1 TEAE. By PT, TEAEs with an incidence rate ≥10% included hyponatraemia, constipation, sinus bradycardia, and urinary tract infection in 3 subjects (42.9%) each; hypokalaemia and pyrexia in 2 subjects (28.6%) each; hyperkalaemia, hyperuricaemia, white blood cell count increased, alanine aminotransferase increased, aspartate aminotransferase increased, C-reactive protein increased, urinary occult blood positive, diarrhoea, functional gastrointestinal disorder, anaemia, cervical vertebral fracture, postoperative delirium, insomnia, deep vein thrombosis, metabolic encephalopathy, and rash in 1 subject each (14.3%).

TABLE 27
Summary of TEAEs by MedDRA System Organ Class and Preferred Term (SS).

| | 3 mg/kg | | | | | ALMB- | | |
|---|---|---|---|---|---|---|---|---|
| | or | | | | | 0166 | Placebo | |
| System Organ Class | 200 mg | 600 mg | 1200 mg | 2400 mg | 4800 mg | Group | Group | Total |
| (SOC) | (N = 4) | (N = 4) | (N = 3) | (N = 3) | (N = 3) | (N = 17) | (N = 7) | (N = 24) |
| Preferred Term (PT) | n (%) | n (%) | n (%) | n (%) | n (%) | n (%) | n (%) | n (%) |
| Subjects with at least | 4 (100) | 4 (100) | 3 (100) | 2 (66.7) | 3 (100) | 16 (94.1) | 7 (100) | 23 (95.8) |
| one TEAE | | | | | | | | |
| Metabolism and | 4 (100) | 3 (75.0) | 0 | 2 (66.7) | 0 | 9 (52.9) | 5 (71.4) | 14 (58.3) |
| nutrition disorders | | | | | | | | |
| Hyponatraemia | 3 (75.0) | 2 (50.0) | 0 | 1 (33.3) | 0 | 6 (35.3) | 3 (42.9) | 9 (37.5) |
| Hypokalaemia | 3 (75.0) | 2 (50.0) | 0 | 0 | 0 | 5 (29.4) | 2 (28.6) | 7 (29.2) |
| Hypoalbuminaemia | 0 | 1 (25.0) | 0 | 1 (33.3) | 0 | 2 (11.8) | 0 | 2 (8.3) |
| Hyperkalaemia | 1 (25.0) | 0 | 0 | 0 | 0 | 1 (5.9) | 1 (14.3) | 2 (8.3) |
| Hypoproteinaemia | 0 | 0 | 0 | 1 (33.3) | 0 | 1 (5.9) | 0 | 1 (4.2) |
| Hypocalcaemia | 0 | 0 | 0 | 1 (33.3) | 0 | 1 (5.9) | 0 | 1 (4.2) |
| Hypochloraemia | 0 | 1 (25.0) | 0 | 0 | 0 | 1 (5.9) | 0 | 1 (4.2) |
| Hyperuricaemia | 0 | 0 | 0 | 0 | 0 | 0 | 1 (14.3) | 1 (4.2) |
| Glucose tolerance | 0 | 0 | 0 | 1 (33.3) | 0 | 1 (5.9) | 0 | 1 (4.2) |
| impaired | | | | | | | | |
| Diabetes mellitus | 0 | 0 | 0 | 1 (33.3) | 0 | 1 (5.9) | 0 | 1 (4.2) |
| Investigations | 2 (50.0) | 4 (100) | 1 (33.3) | 2 (66.7) | 0 | 9 (52.9) | 3 (42.9) | 12 (50.0) |
| White blood cell | 1 (25.0) | 1 (25.0) | 0 | 1 (33.3) | 0 | 3 (17.6) | 1 (14.3) | 4 (16.7) |
| count increased | | | | | | | | |
| Alanine | 0 | 2 (50.0) | 0 | 1 (33.3) | 0 | 3 (17.6) | 1 (14.3) | 4 (16.7) |
| aminotransferase | | | | | | | | |
| increased | | | | | | | | |
| Aspartate | 0 | 2 (50.0) | 0 | 1 (33.3) | 0 | 3 (17.6) | 1 (14.3) | 4 (16.7) |
| aminotransferase | | | | | | | | |
| increased | | | | | | | | |
| C-reactive protein | 1 (25.0) | 1 (25.0) | 0 | 0 | 0 | 2 (11.8) | 1 (14.3) | 3 (12.5) |
| increased | | | | | | | | |
| Red blood cells | 1 (25.0) | 1 (25.0) | 0 | 1 (33.3) | 0 | 3 (17.6) | 0 | 3 (12.5) |
| urine positive | | | | | | | | |
| Urinary occult | 1 (25.0) | 1 (25.0) | 0 | 0 | 0 | 2 (11.8) | 1 (14.3) | 3 (12.5) |
| blood positive | | | | | | | | |
| Fibrin D dimer | 2 (50.0) | 1 (25.0) | 0 | 0 | 0 | 3 (17.6) | 0 | 3 (12.5) |
| increased | | | | | | | | |
| Glucose urine | 1 (25.0) | 0 | 1 (33.3) | 0 | 0 | 2 (11.8) | 0 | 2 (8.3) |
| present | | | | | | | | |
| Red blood cell | 1 (25.0) | 0 | 0 | 0 | 0 | 1 (5.9) | 0 | 1 (4.2) |
| sedimentation rate | | | | | | | | |
| increased | | | | | | | | |
| Reticulocyte | 1 (25.0) | 0 | 0 | 0 | 0 | 1 (5.9) | 0 | 1 (4.2) |
| percentage increased | | | | | | | | |
| Reticulocyte count | 1 (25.0) | 0 | 0 | 0 | 0 | 1 (5.9) | 0 | 1 (4.2) |
| increased | | | | | | | | |
| Blood creatine | 0 | 0 | 0 | 1 (33.3) | 0 | 1 (5.9) | 0 | 1 (4.2) |
| phosphokinase | | | | | | | | |
| increased | | | | | | | | |
| Blood lactate | 0 | 0 | 0 | 1 (33.3) | 0 | 1 (5.9) | 0 | 1 (4.2) |
| dehydrogenase | | | | | | | | |
| increased | | | | | | | | |
| Blood fibrinogen | 1 (25.0) | 0 | 0 | 0 | 0 | 1 (5.9) | 0 | 1 (4.2) |
| increased | | | | | | | | |
| Platelet count | 0 | 1 (25.0) | 0 | 0 | 0 | 1 (5.9) | 0 | 1 (4.2) |
| decreased | | | | | | | | |
| Neutrophil count | 0 | 0 | 0 | 1 (33.3) | 0 | 1 (5.9) | 0 | 1 (4.2) |
| increased | | | | | | | | |
| Gastrointestinal | 1 (25.0) | 2 (50.0) | 1 (33.3) | 0 | 3 (100) | 7 (41.2) | 4 (57.1) | 11 (45.8) |
| disorders | | | | | | | | |
| Constipation | 1 (25.0) | 1 (25.0) | 1 (33.3) | 0 | 2 (66.7) | 5 (29.4) | 3 (42.9) | 8 (33.3) |
| Diarrhoea | 0 | 1 (25.0) | 0 | 0 | 1 (33.3) | 2 (11.8) | 1 (14.3) | 3 (12.5) |
| Functional | 0 | 0 | 0 | 0 | 0 | 0 | 1 (14.3) | 1 (4.2) |
| gastrointestinal | | | | | | | | |
| disorder | | | | | | | | |
| Neurogenic bowel | 0 | 1 (25.0) | 0 | 0 | 0 | 1 (5.9) | 0 | 1 (4.2) |
| General disorders and | 1 (25.0) | 1 (25.0) | 2 (66.7) | 0 | 2 (66.7) | 6 (35.3) | 2 (28.6) | 8 (33.3) |
| administration site | | | | | | | | |
| conditions | | | | | | | | |
| Pyrexia | 1 (25.0) | 1 (25.0) | 1 (33.3) | 0 | 2 (66.7) | 5 (29.4) | 2 (28.6) | 7 (29.2) |
| Chest discomfort | 0 | 0 | 1 (33.3) | 0 | 0 | 1 (5.9) | 0 | 1 (4.2) |
| Cardiac disorders | 0 | 1 (25.0) | 0 | 1 (33.3) | 1 (33.3) | 3 (17.6) | 3 (42.9) | 6 (25.0) |
| Sinus bradycardia | 0 | 1 (25.0) | 0 | 0 | 1 (33.3) | 2 (11.8) | 3 (42.9) | 5 (20.8) |
| Atrial fibrillation | 0 | 0 | 0 | 1 (33.3) | 0 | 1 (5.9) | 0 | 1 (4.2) |
| Arrhythmia | 0 | 1 (25.0) | 0 | 0 | 0 | 1 (5.9) | 0 | 1 (4.2) |
| Bundle branch | 0 | 1 (25.0) | 0 | 0 | 0 | 1 (5.9) | 0 | 1 (4.2) |
| block right | | | | | | | | |
| Blood and lymphatic | 1 (25.0) | 3 (75.0) | 0 | 1 (33.3) | 0 | 5 (29.4) | 1 (14.3) | 6 (25.0) |
| system disorders | | | | | | | | |
| Anaemia | 1 (25.0) | 3 (75.0) | 0 | 1 (33.3) | 0 | 5 (29.4) | 1 (14.3) | 6 (25.0) |
| Infections and | 0 | 1 (25.0) | 0 | 1 (33.3) | 0 | 2 (11.8) | 3 (42.9) | 5 (20.8) |
| infestations | | | | | | | | |
| Urinary tract | 0 | 1 (25.0) | 0 | 1 (33.3) | 0 | 2 (11.8) | 3 (42.9) | 5 (20.8) |
| infection | | | 0 | | | | | 1 (4.2) |
| COVID-19 | 0 | 1 (25.0) | | 0 | 0 | 1 (5.9) | 0 | |
| Wound infection | 0 | 1 (25.0) | 0 | 0 | 0 | 1 (5.9) | 0 | 1 (4.2) |
| staphylococcal | | | | | | | | |
| Upper respiratory | 0 | 0 | 0 | 1 (33.3) | 0 | 1 (5.9) | 0 | 1 (4.2) |
| tract infection | | | | | | | | |
| Injury, poisoning and | 1 (25.0) | 0 | 1 (33.3) | 1 (33.3) | 0 | 3 (17.6) | 1 (14.3) | 4 (16.7) |
| procedural | | | | | | | | |
| complications | | | | | | | | |
| Procedural pain | 0 | 0 | 1 (33.3) | 1 (33.3) | 0 | 2 (11.8) | 0 | 2 (8.3) |
| Cervical vertebral | 0 | 0 | 0 | 0 | 0 | 0 | 1 (14.3) | 1 (4.2) |
| fracture | | | | | | | | |
| Infusion related | 1 (25.0) | 0 | 0 | 0 | 0 | 1 (5.9) | 0 | 1 (4.2) |
| reactions | | | | | | | | |
| Postoperative | 0 | 0 | 0 | 0 | 0 | 0 | 1 (14.3) | 1 (4.2) |
| delirium | | | | | | | | |
| Respiratory, thoracic | 0 | 0 | 2 (66.7) | 0 | 1 (33.3) | 3 (17.6) | 0 | 3 (12.5) |
| and mediastinal | | | | | | | | |
| disorders | | | | | | | | |
| Pneumonitis | 0 | 0 | 0 | 0 | 1 (33.3) | 1 (5.9) | 0 | 1 (4.2) |
| Respiratory failure | 0 | 0 | 0 | 0 | 1 (33.3) | 1 (5.9) | 0 | 1 (4.2) |
| Oropharyngeal | 0 | 0 | 1 (33.3) | 0 | 0 | 1 (5.9) | 0 | 1 (4.2) |
| pain | | | | | | | | |
| Productive cough | 0 | 0 | 1 (33.3) | 0 | 0 | 1 (5.9) | 0 | 1 (4.2) |
| Psychiatric disorders | 1 (25.0) | 0 | 0 | 0 | 1 (33.3) | 2 (11.8) | 1 (14.3) | 3 (12.5) |
| Insomnia | 1 (25.0) | 0 | 0 | 0 | 0 | 1 (5.9) | 1 (14.3) | 2 (8.3) |
| Delirium | 0 | 0 | 0 | 0 | 1 (33.3) | 1 (5.9) | 0 | 1 (4.2) |
| Vascular disorders | 0 | 1 (25.0) | 0 | 0 | 1 (33.3) | 2 (11.8) | 1 (14.3) | 3 (12.5) |
| Deep vein | 0 | 1 (25.0) | 0 | 0 | 1 (33.3) | 2 (11.8) | 1 (14.3) | 3 (12.5) |
| thrombosis | | | | | | | | |
| Nervous system | 0 | 0 | 0 | 0 | 0 | 0 | 1 (14.3) | 1 (4.2) |
| disorders | | | | | | | | |
| Metabolic | 0 | 0 | 0 | 0 | 0 | 0 | 1 (14.3) | 1 (4.2) |
| encephalopathy | | | | | | | | |
| Musculoskeletal and | 0 | 0 | 1 (33.3) | 0 | 0 | 1 (5.9) | 0 | 1 (4.2) |
| connective tissue | | | | | | | | |
| disorders | | | | | | | | |
| Neck pain | 0 | 0 | 1 (33.3) | 0 | 0 | 1 (5.9) | 0 | 1 (4.2) |
| Skin and | 0 | 0 | 0 | 0 | 0 | 0 | 1 (14.3) | 1 (4.2) |
| subcutaneous tissue | | 0 | | | | | | |
| disorders | | | | | | | | |
| Rash | 0 | | 0 | 0 | 0 | 0 | 1 (14.3) | 1 (4.2) |
| Renal and urinary | 0 | 1 (25.0) | 0 | 0 | 0 | 1 (5.9) | 0 | 1 (4.2) |
| disorders | | | | | | | | |
| Neurogenic | 0 | 1 (25.0) | 0 | 0 | 0 | 1 (5.9) | 0 | 1 (4.2) |
| bladder | | | | | | | | |

Abbreviations: TEAE = Treatment-emergent adverse event; COVID-19 = novel coronavirus pneumonia pandemic.
Note:
TEAE: Events that occur during the treatment period that did not appear before treatment or worsened compared to before treatment; percentages were calculated based on the number of subjects in the SS for each group; AEs were coded using MedDRA version 27.0.

### 3. Analysis of Adverse Events

a. Relationship Between Adverse Events and Study Intervention

TEAEs related to the study drug by SOC and PT are shown in Table 28.

Based on the SS, in the ALMB-0166 group (N=17), 6 subjects (35.3%) experienced at least 1 TEAE related to the study drug, including sinus bradycardia, atrial fibrillation, alanine aminotransferase increased, red blood cells urine positive, urinary occult blood positive, glucose urine present, aspartate aminotransferase increased, reticulocyte percentage increased, reticulocyte count increased, and infusion related reaction, each occurring in 1 subject (5.9%).

In the placebo group (N=7), 2 subjects (28.6%) experienced at least 1 TEAE related to the study drug, both of which were sinus bradycardia.

TABLE 28
Summary of TEAEs Related to the Study Drug by MedDRA
System Organ Class and Preferred Term (SS).

| | 3 mg/kg | | | | | ALMB- | | |
|---|---|---|---|---|---|---|---|---|
| | or | | | | | 0166 | Placebo | |
| System Organ Class | 200 mg | 600 mg | 1200 mg | 2400 mg | 4800 mg | Group | Group | Total |
| (SOC) | (N = 4) | (N = 4) | (N = 3) | (N = 3) | (N = 3) | (N = 17) | (N = 7) | (N = 24) |
| Preferred Term (PT) | n (%) | n (%) | n (%) | n (%) | n (%) | n (%) | n (%) | n (%) |
| Subjects with at least | 2 (50.0) | 0 | 1 (33.3) | 2 (66.7) | 1 (33.3) | 6 (35.3) | 2 (28.6) | 8 (33.3) |
| one TEAE | | | | | | | | |
| Cardiac disorders | 0 | 0 | 0 | 1 (33.3) | 1 (33.3) | 2 (11.8) | 2 (28.6) | 4 (16.7) |
| Sinus bradycardia | 0 | 0 | 0 | 0 | 1 (33.3) | 1 (5.9) | 2 (28.6) | 3 (12.5) |
| Atrial fibrillation | 0 | 0 | 0 | 1 (33.3) | 0 | 1 (5.9) | 0 | 1 (4.2) |
| Investigations | 1 (25.0) | 0 | 1 (33.3) | 1 (33.3) | 0 | 3 (17.6) | 0 | 3 (12.5) |
| Alanine | 0 | 0 | 0 | 1 (33.3) | 0 | 1 (5.9) | 0 | 1 (4.2) |
| aminotransferase | | | | | | | | |
| increased | | | | | | | | |
| Red blood cells | 1 (25.0) | 0 | 0 | 0 | 0 | 1 (5.9) | 0 | 1 (4.2) |
| urine positive | | | | | | | | |
| Urinary occult blood | 1 (25.0) | 0 | 0 | 0 | 0 | 1 (5.9) | 0 | 1 (4.2) |
| positive | | | | | | | | |
| Glucose urine | 0 | 0 | 1 (33.3) | 0 | 0 | 1 (5.9) | 0 | 1 (4.2) |
| present | | | | | | | | |
| Aspartate | 0 | 0 | 0 | 1 (33.3) | 0 | 1 (5.9) | 0 | 1 (4.2) |
| aminotransferase | | | | | | | | |
| increased | | | | | | | | |
| Reticulocyte | 1 (25.0) | 0 | 0 | 0 | 0 | 1 (5.9) | 0 | 1 (4.2) |
| percentage increased | | | | | | | | |
| Reticulocyte count | 1 (25.0) | 0 | 0 | 0 | 0 | 1 (5.9) | 0 | 1 (4.2) |
| increased | | | | | | | | |
| Injury, poisoning and | 1 (25.0) | 0 | 0 | 0 | 0 | 1 (5.9) | 0 | 1 (4.2) |
| procedural | | | | | | | | |
| complications | | | | | | | | |
| Infusion related | 1 (25.0) | 0 | 0 | 0 | 0 | 1 (5.9) | 0 | 1 (4.2) |
| reactions | | | | | | | | |

Abbreviation: TEAE = Treatment-emergent adverse event.
Note:
TEAE: Events that occurred during treatment that did not appear before treatment or worsened compared to before treatment; percentages were calculated based on the number of subjects in the SS for each group; related to the study drug: causal relationship with the study drug is definitely related, probably related, or possibly related. AEs were coded using MedDRA version 27.0.

b. Classification of Adverse Event Severity

TEAEs of CTCAE Grade ≥3 by SOC and PT are shown in Table 29.

Based on the SS, in the ALMB-0166 group (N=17), the numbers of subjects who experienced CTCAE Grades 1, 2, 3, 4, and 5 TEAEs were 7 subjects (41.2%), 6 subjects (35.3%), 2 subjects (11.8%), 1 subject (5.9%), and 0, respectively. Three subjects (17.6%) experienced CTCAE Grade ≥3 TEAEs, including 1 subject (5.9%) each of hypokalaemia, pneumonitis, respiratory failure, and deep vein thrombosis.

In the placebo group (N=7), the numbers of subjects who experienced CTCAE Grades 1, 2, 3, 4, and 5 TEAEs were 1 subject (14.3%), 3 subjects (42.9%), 3 subjects (42.9%), 0, and 0, respectively. Three subjects (42.9%) experienced CTCAE Grade ≥3 TEAEs, including hypokalemia in 2 subjects (28.6%) and hyponatremia in 1 subject (14.3%).

The TEAEs related to the study drug that occurred in both the ALMB-0166 group and the placebo group were all of CTCAE Grade 1.

TABLE 29
Summary of TEAEs of CTCAE Grade ≥3 by MedDRA System Organ Class and Preferred Term (SS).

| | | | | | | | ALMB- | | |
|---|---|---|---|---|---|---|---|---|---|
| | | 3 mg/kg | | | | | 0166 | Placebo | |
| System Organ Class | | or 200 mg | 600 mg | 1200 mg | 2400 mg | 4800 mg | Group | Group | Total |
| (SOC) | CTCAE | (N = 4) | (N = 4) | (N = 3) | (N = 3) | (N = 3) | (N = 17) | (N = 7) | (N = 24) |
| Preferred Term (PT) | Grade | n (%) | n (%) | n (%) | n (%) | n (%) | n (%) | n (%) | n (%) |
| Subjects with at least | Grade 3 | 0 | 2 (50.0) | 0 | 0 | 0 | 2 (11.8) | 3 (42.9) | 5 (20.8) |
| one TEAE | Grade 4 | 0 | 0 | 0 | 0 | 1 (33.3) | 1 (5.9) | 0 | 1 (4.2) |
| | Grade 5 | 0 | 0 | 0 | 0 | 0 | 0 | 0 | 0 |
| Metabolism and | Grade 3 | 0 | 1 (25.0) | 0 | 0 | 0 | 1 (5.9) | 3 (42.9) | 4 (16.7) |
| nutrition disorders | Grade 4 | 0 | 0 | 0 | 0 | 0 | 0 | 0 | 0 |
| | Grade 5 | 0 | 0 | 0 | 0 | 0 | 0 | 0 | 0 |
| Hyponatraemia | Grade 3 | 0 | 0 | 0 | 0 | 0 | 0 | 1 (14.3) | 1 (4.2) |
| | Grade 4 | 0 | 0 | 0 | 0 | 0 | 0 | 0 | 0 |
| | Grade 5 | 0 | 0 | 0 | 0 | 0 | 0 | 0 | 0 |
| Hypokalaemia | Grade 3 | 0 | 1 (25.0) | 0 | 0 | 0 | 1 (5.9) | 2 (28.6) | 3 (12.5) |
| | Grade 4 | 0 | 0 | 0 | 0 | 0 | 0 | 0 | 0 |
| | Grade 5 | 0 | 0 | 0 | 0 | 0 | 0 | 0 | 0 |
| Respiratory, thoracic | Grade 3 | 0 | 0 | 0 | 0 | 0 | 0 | 0 | 0 |
| and mediastinal | Grade 4 | 0 | 0 | 0 | 0 | 1 (33.3) | 1 (5.9) | 0 | 1 (4.2) |
| disorders | Grade 5 | 0 | 0 | 0 | 0 | 0 | 0 | 0 | 0 |
| Pneumonitis | Grade 3 | 0 | 0 | 0 | 0 | 1 (33.3) | 1 (5.9) | 0 | 1 (4.2) |
| | Grade 4 | 0 | 0 | 0 | 0 | 0 | 0 | 0 | 0 |
| | Grade 5 | 0 | 0 | 0 | 0 | 0 | 0 | 0 | 0 |
| Respiratory failure | Grade 3 | 0 | 0 | 0 | 0 | 0 | 0 | 0 | 0 |
| | Grade 4 | 0 | 0 | 0 | 0 | 1 (33.3) | 1 (5.9) | 0 | 1 (4.2) |
| | Grade 5 | 0 | 0 | 0 | 0 | 0 | 0 | 0 | 0 |
| Vascular disorders | Grade 3 | 0 | 1 (25.0) | 0 | 0 | 0 | 1 (5.9) | 0 | 1 (4.2) |
| | Grade 4 | 0 | 0 | 0 | 0 | 0 | 0 | 0 | 0 |
| | Grade 5 | 0 | 0 | 0 | 0 | 0 | 0 | 0 | 0 |
| Deep vein thrombosis | Grade 3 | 0 | 1 (25.0) | 0 | 0 | 0 | 1 (5.9) | 0 | 1 (4.2) |
| | Grade 4 | 0 | 0 | 0 | 0 | 0 | 0 | 0 | 0 |
| | Grade 5 | 0 | 0 | 0 | 0 | 0 | 0 | 0 | 0 |

Abbreviation: TEAE = Treatment-emergent adverse event.
Note:
TEAE: Events that occurred during the treatment period that did not appear before treatment or worsened compared to before treatment; percentages were calculated based on the number of subjects in the SS for each group; AEs were coded using MedDRA version 27.0.

c. Serious Adverse Event

SAEs by SOC and PT are presented in Table 30. Based on the SS, only 1 subject (5.9%) in the ALMB-0166 group experienced an SAE, with PT of deep vein thrombus, determined to be unlikely related to the study drug. No subjects experienced SAEs related to the study drug.

TABLE 30
Summary of SAEs by MedDRA System Organ Class and Preferred Term (SS).

| | 3 mg/kg | | | | | ALMB- | | |
|---|---|---|---|---|---|---|---|---|
| | or | | | | | 0166 | Placebo | |
| | 200 mg | 600 mg | 1200 mg | 2400 mg | 4800 mg | Group | Group | Total |
| System Organ Class | (N = 4) | (N = 4) | (N = 3) | (N = 3) | (N = 3) | (N = 17) | (N = 7) | (N = 24) |
| (SOC) Preferred Term (PT) | n (%) | n (%) | n (%) | n (%) | n (%) | n (%) | n (%) | n (%) |
| Subjects with at least | 0 | 1 (25.0) | 0 | 0 | 0 | 1 (5.9) | 0 | 1 (4.2) |
| one TEAE | | | | | | | | |
| Vascular disorders | 0 | 1 (25.0) | 0 | 0 | 0 | 1 (5.9) | 0 | 1 (4.2) |
| Deep vein | 0 | 1 (25.0) | 0 | 0 | 0 | 1 (5.9) | 0 | 1 (4.2) |
| thrombosis | | | | | | | | |

Abbreviation: TEAE = Treatment-emergent adverse event.
Note:
TEAE: Events that occurred during the treatment period that did not appear before treatment or worsened compared to before treatment; percentages were calculated based on the number of subjects in the SS for each group; AEs were coded using MedDRA version 27.0.

### C. Clinical Laboratory Evaluation

### 1. Evaluation of Laboratory Test Indicators

a. Hematology

In the 200 mg (or 3 mg/kg) group, hematology indicators that were normal or abnormal not clinically significant at baseline and became abnormal with clinical significance after treatment as the worst result included: red blood cell count and reticulocyte in 1 subject each (25.0%).

In the 600 mg group, hematology indicators that were normal or abnormal not clinically significant at baseline and became abnormal with clinical significance after treatment as the worst result included: haemoglobin and red blood cell count in 3 subjects each (75.0%), haematocrit in 2 subjects (50.0%), and neutrophil count and white blood cell count in 1 subject each (25.0%).

In the 1200 mg, 2400 mg, and 4800 mg groups, no subjects had hematology indicators that were normal or abnormal not clinically significant at baseline and became abnormal with clinical significance after treatment as the worst result.

In the placebo group, hematology indicators that were normal or abnormal not clinically significant at baseline and became abnormal with clinical significance after treatment as the worst result included: red blood cell count in 2 subjects (28.6%), and hematocrit, haemoglobin, and white blood cell count in 1 subject each (14.3%).

b. Blood Chemistry

In the 200 mg (or 3 mg/kg) group, blood chemistry indicators that were normal or abnormal not clinically significant at baseline and became abnormal with clinical significance after treatment as the worst result included: sodium in 3 subjects (75.0%) and potassium in 2 subjects (50.0%).

In the 600 mg group, blood chemistry indicators that were normal or abnormal not clinically significant at baseline and became abnormal with clinical significance after treatment as the worst result included: alanine aminotransferase, aspartate aminotransferase, potassium, and sodium in 2 subjects each (50.0%), and albumin, cl, protein, and globulin in 1 subject each (25.0%).

In the 2400 mg group, blood chemistry indicators that were normal or abnormal not clinically significant at baseline and became abnormal with clinical significance after treatment as the worst result included: albumin, alanine aminotransferase, aspartate aminotransferase, globulin, protein, sodium, and lactate dehydrogenase in 1 subject each (33.3%).

In the 1200 mg and 4800 mg groups, no subjects had blood biochemistry parameters that were normal or abnormal not clinically significant at baseline and became abnormal with clinical significance after treatment as the worst result.

In the placebo group, blood chemistry indicators that were normal or abnormal not clinically significant at baseline and became abnormal with clinical significance after treatment as the worst result included: potassium in 2 subjects (28.6%), and alanine aminotransferase, aspartate aminotransferase, cholesterol total, sodium, and glucose in 1 subject each (14.3%).

c. Urinalysis

In the 200 mg (or 3 mg/kg) group, urinalysis indicators that were normal or abnormal not clinically significant at baseline and became abnormal with clinical significance after treatment as the worst result included: urine occult blood and red blood cells urine in 1 subject each (25.0%).

In the 600 mg group, urinalysis indicators that were normal or abnormal not clinically significant at baseline and became abnormal with clinical significance after treatment as the worst result included: urine occult blood, protein urine, red blood cells urine, and urobilinogen in 1 subject each (25.0%).

In the 1200 mg group, urinalysis indicators that were normal or abnormal not clinically significant at baseline and became abnormal with clinical significance after treatment as the worst result included: urinary glucose in 1 subject (33.3%).

In the 2400 mg group, urinalysis indicators that were normal or abnormal not clinically significant at baseline and became abnormal with clinical significance after treatment as the worst result included: red blood cells urine in 2 subjects (66.7%), and urinary glucose, protein urine, white blood cells urine, and urinary nitrites in 1 subject each (33.3%).

In the 4800 mg group, no subjects had urinalysis parameters that were normal or abnormal not clinically significant at baseline and became abnormal with clinical significance after treatment as the worst result.

In the placebo group, urinalysis indicators that were normal or abnormal not clinically significant at baseline and became abnormal with clinical significance after treatment as the worst result included: white blood cells urine in 3 subjects (50.0%), and glucose, urine ketone body, nitrite urine, urine occult blood, red blood cells urine, specific gravity, and urine pH in 1 subject each (16.7%).

d. Coagulation Function

In the ALMB-0166 group and the placebo group, no subjects had coagulation function indicators that were normal or abnormal not clinically significant at baseline and became abnormal with clinical significance after treatment as the worst result.

e. Troponin

In both the ALMB-0166 group and the placebo group, no subjects had troponin indicators that were normal or abnormal not clinically significant at baseline and became abnormal with clinical significance after treatment as the worst result.

### D. Vital Signs, Physical Findings, and Other Observations Related to Safety

### 1. Physical Examination

In the ALMB-0166 group and the placebo group, no subjects had physical examination indicators that were normal or abnormal not clinically significant at baseline and became abnormal with clinical significance after treatment as the worst result.

### 2. Electrocardiogram

a. Values of ECG Changes Over Time

The mean (standard deviation) of changes from baseline in minimum heart rate after baseline in each group (200 mg [or 3 mg/kg] group, 600 mg group, 1200 mg group, 2400 mg group, 4800 mg group, and placebo group) were −11.8 (8.66), −13.8 (3.77), −12.7 (10.02), −15.0 (13.53), −7.7 (8.08), and −23.9 (8.30), respectively.

The mean (standard deviation) of changes from baseline in maximum heart rate after baseline in each group was 17.3 (10.66), 3.3 (2.22), 3.7 (8.50), 9.0 (13.53), 12.3 (10.21), and −2.9 (8.78), respectively.

The mean (standard deviation) of changes from baseline in minimum PR interval after baseline in each group was −19.0 (10.89), −9.0 (11.14), 1.3 (6.43), −26.3 (12.34), −10.0 (2.65), and −31.3 (15.35), respectively.

The mean (standard deviation) of changes from baseline in maximum PR interval after baseline in each group was 13.5 (10.34), 17.8 (17.75), 19.3 (9.87), −2.7 (9.29), 10.7 (7.51), and 1.6 (14.66), respectively.

The mean (standard deviation) of changes from baseline in minimum QRS interval after baseline in each group was −23.3 (20.97), −10.5 (5.26), −6.7 (1.15), −0.7 (3.06), −12.3 (2.52), and −6.1 (5.27), respectively.

The mean (standard deviation) of changes from baseline in maximum QRS interval after baseline in each group was 2.8 (9.36), 11.3 (16.76), 6.0 (2.00), 19.3 (14.47), 2.7 (1.53), and 10.5 (4.68), respectively.

The mean (standard deviation) of changes from baseline in minimum QT interval after baseline in each group was −48.0 (31.40), −5.8 (17.02), −17.3 (28.10), −32.7 (39.31), −48.0 (33.42), and −9.4 (21.11), respectively.

The mean (standard deviation) of changes from baseline in maximum QT interval after baseline in each group was 25.0 (24.58), 47.8 (29.40), 32.7 (17.01), 40.3 (42.57), 28.3 (13.61), and 60.4 (29.38), respectively.

The mean (standard deviation) of changes from baseline in minimum QTcF interval after baseline in each group was −30.50 (17.020), −17.50 (12.261), −26.70 (1.572), −26.07 (11.243), −28.27 (9.886), and −25.60 (17.949), respectively.

The mean (standard deviation) of changes from baseline in maximum QTcF interval after baseline in each group was 5.75 (16.378), 29.25 (26.600), 11.57 (3.262), 10.60 (14.124), 17.47 (5.575), and 18.83 (29.889), respectively.

b. ECG Abnormalities

In the 600 mg group, 2400 mg group, and 4800 mg group, 1 subject each (25.0%) had ECG results that were normal or abnormal not clinically significant at baseline and became abnormal with clinical significance after treatment as the worst result. In the remaining ALMB- 0166 groups, no subjects had ECG results that were normal or abnormal not clinically significant at baseline and became abnormal with clinical significance after treatment as the worst result.

In the placebo group, 3 subjects (42.9%) had ECG results that were normal or abnormal not clinically significant at baseline and became abnormal with clinical significance after treatment as the worst result.

### 3. Pulse Oxygen Saturation

In the 1200 mg group, 1 subject (33.3%) had normal pulse oxygen saturation at baseline, with the worst post-treatment result being abnormal and clinically significant.

In the 4800 mg group, 2 subjects (66.7%) had normal pulse oxygen saturation at baseline, with the worst post-treatment result being abnormal and clinically significant.

In the placebo group, 2 subjects (28.6%) had pulse oxygen saturation results that were normal or abnormal not clinically significant at baseline and became abnormal with clinical significance after treatment as the worst result.

### 4. Imaging Examination

Only in the placebo group, 2 subjects (50.0%) had CT scans showing baseline abnormal but not clinically significant, which shifted to abnormal and clinically significant as the worst post-treatment result.

No imaging investigations were observed in the other groups where baseline abnormalities of no clinical significance shifted into abnormalities of clinical significance as the worst post-treatment result.

### 5. Electrophysiological Examination

Only 1 subject in the placebo group underwent electrophysiological examination. The electromyogram-terminal latency and electromyogram-nerve conduction velocity were normal at baseline but became abnormal with clinical significance after treatment as the worst result.

### E. Immunogenicity Evaluation

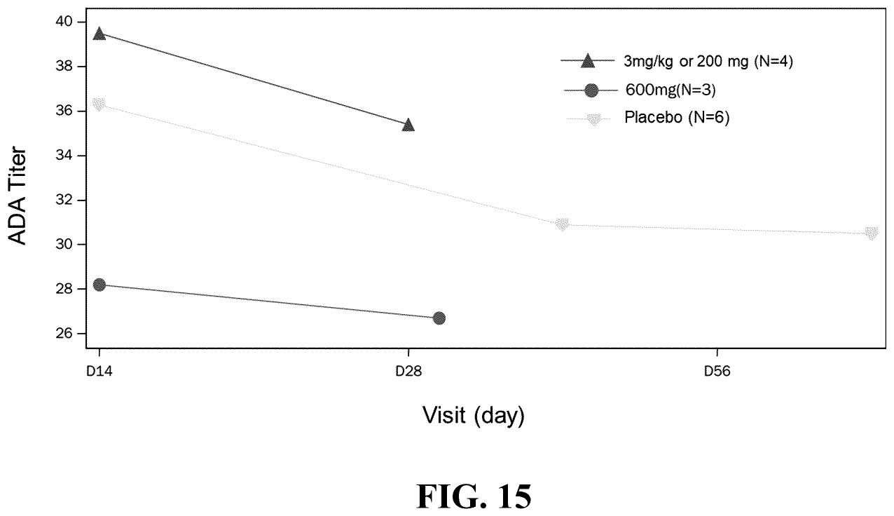

ADA analyses are provided in Table 31. The mean titer-time curve is shown in FIG. 15.

Based on the IS, only in the 200 mg (or 3 mg/kg) group, 600 mg group, and placebo group, 1 subject each was ADA-negative at baseline and had at least one ADA-positive sample after baseline.

In the 200 mg (or 3 mg/kg) group, 1 subject (25.0%) was ADA-negative at baseline and transiently ADA-positive after baseline. The time to first ADA-positive occurrence was 14.0 days, lasting 12.0 days, with a peak titer of 39.5.

In the 600 mg group, 1 subject (33.3%) was ADA-negative at baseline and ADA-positive after baseline. The time to first ADA-positive occurrence was 15.0 days, lasting 45.0 days, with a peak titer of 28.2.

In the placebo group, 1 subject (16.7%) was ADA-negative at baseline and ADA-positive after baseline. The time to first ADA-positive occurrence was 14.0 days, lasting for 36.0 days, with a peak titer of 36.3.

The above results indicated that after drug therapies, the incidence of ADA-positive among subjects was low, with no obvious dose relationship, and the titers of ADA-positive samples were low. Therefore, the immunogenicity risk of ALMB-0166 is low.

TABLE 31
ADA Analysis (IS).

| | 3 mg/kg or 200 mg | 600 mg | 1200 mg | 2400 mg |
|---|---|---|---|---|
| | (N = 4) | (N = 3) | (N = 3) | (N = 2) |
| Number and percentage of subjects with at | 1 (25.0) | 1 (33.3) | 0 | 0 |
| least one ADA-positive sample at or after | | | | |
| baseline, n (%) | | | | |
| Baseline ADA analysis | | | | |
| Number and percentage of subjects with a | 0 | 0 | 0 | 0 |
| positive baseline, n (%) | | | | |
| Number and percentage of ADA-positive | 1 (25.0) | 1 (33.3) | 0 | 0 |
| subjects^{[1]}, n (%) | | | | |
| Number and percentage of subjects with | 1 (25.0) | 1 (33.3) | 0 | 0 |
| negative baseline ADA and positive ADA | | | | |
| after baseline, n (%) | | | | |
| Number and percentage of subjects with | 1 (25.0) | 0 | 0 | 0 |
| transient positivity after baseline^{[2]}, n (%) | | | | |
| Number and percentage of subjects with | 0 | 0 | 0 | 0 |
| persistent positivity after baseline^{[3]}, n (%) | | | | |
| Number and percentage of other positive | 0 | 1 (33.3) | 0 | 0 |
| subjects^{[4]}, n (%) | | | | |
| Time to first ADA positivity^{[5]} (days) | | | | |
| Mean (standard deviation) | 14.00 (—) | 15.00 (—) | — | — |
| ADA-positive duration^{[6]} (days) | | | | |
| Mean (standard deviation) | 12.00 (—) | 45.00 (—) | — | — |
| Peak titer | | | | |
| Mean (standard deviation) | 39.50 (—) | 28.20 (—) | — | — |
| Number and percentage of ADA-negative | 3 (75.0) | 2 (66.7) | 3 (100) | 2 (100) |
| subjects^{[7]}, n (%) | | | | |
| Number and percentage of subjects with all | 3 (75.0) | 2 (66.7) | 3 (100) | 2 (100) |
| samples being ADA-negative at baseline and | | | | |
| after baseline, n (%) | | | | |
| Number and percentage of subjects who | 0 | 0 | 0 | 0 |
| were ADA-positive at baseline and negative | | | | |
| after baseline or had a subsequent | | | | |
| titer/baseline titer <4, n (%) | | | | |

| | | Placebo | |
|---|---|---|---|
| | 4800 mg | Group | Total |
| | (N = 3) | (N = 6) | (N = 21) |
| Number and percentage of subjects with at | 0 | 1 (16.7) | 3 (14.3) |
| least one ADA-positive sample at or after | | | |
| baseline, n (%) | | | |
| Baseline ADA analysis | | | |
| Number and percentage of subjects with a | 0 | 0 | 0 |
| positive baseline, n (%) | | | |
| Number and percentage of ADA-positive | 0 | 1 (16.7) | 3 (14.3) |
| subjects^{[1]}, n (%) | | | |
| Number and percentage of subjects with | 0 | 1 (16.7) | 3 (14.3) |
| negative baseline ADA and positive ADA | | | |
| after baseline, n (%) | | | |
| Number and percentage of subjects with | 0 | 0 | 1 (4.8) |
| transient positivity after baseline^{[2]}, n (%) | | | |
| Number and percentage of subjects with | 0 | 0 | 0 |
| persistent positivity after baseline^{[3]}, n (%) | | | |
| Number and percentage of other positive | 0 | 1 (16.7) | 2 (9.5) |
| subjects^{[4]}, n (%) | | | |
| Time to first ADA positivity^{[5]} (days) | | | |
| Mean (standard deviation) | — | 14.00 (—) | 14.33 (0.577) |
| ADA-positive duration^{[6]} (days) | | | |
| Mean (standard deviation) | — | 36.00 (—) | 31.00 (17.059) |
| Peak titer | | | |
| Mean (standard deviation) | — | 36.30 (—) | 34.67 (5.824) |
| Number and percentage of ADA-negative | 3 (100) | 5 (83.3) | 18 (85.7) |
| subjects^{[7]}, n (%) | | | |
| Number and percentage of subjects with all | 3 (100) | 5 (83.3) | 18 (85.7) |
| samples being ADA-negative at baseline and | | | |
| after baseline, n (%) | | | |
| Number and percentage of subjects who | 0 | 0 | 0 |
| were ADA-positive at baseline and negative | | | |
| after baseline or had a subsequent | | | |
| titer/baseline titer <4, n (%) | | | |

Note:
ADA = Anti-drug antibody, n = number of subjects meeting specific criteria, N = number of subjects in the analysis population.
^{[1]}ADA-positive subjects included: subjects who were ADA-negative at baseline and ADA-positive after baseline, and subjects who were ADA-positive at baseline with any post-baseline titer/baseline titer ≥4(9).
^{[2]}The analysis was based on treatment-emergent ADA-positive subjects, including subjects whose last ADA sample was negative and 1) had only one ADA-positive sample during treatment; 2) two or more ADA-positive samples during treatment, with the first ADA-positive sample being less than 16 weeks from the last ADA-positive sample.
^{[3]}The analysis was based on treatment-emergent ADA-positive subjects. Subjects who had two or more ADA-positive samples observed during the treatment period, and in whom the time between the first ADA-positive sample and the last ADA-positive sample was ≥16 weeks.
^{[4]}Cases not classified as transient and persistent positive subjects.
^{[5]}The time was the date of the first ADA-positive visit − the date of first drug administration + 1.
^{[6]}The time was the date of the last ADA-positive visit − the date of the first ADA-positive visit + 1.
^{[7]}ADA-negative subjects refer to subjects without treatment-induced or -enhanced ADA-positive samples.

### F. Safety Conclusions

25 patients with C3-C7 SCI were enrolled and 24 patients received treatment with investigational drugs (17 with ALMB-0166, 7 with placebo). Treatment emergent adverse events (TEAEs) occurred in 94.1% (16/17) and 100% (7/7) of ALMB-0166 and placebo treated patients, respectively. TEAEs of grade 3 were 17.6% (3/17) with ALMB-0166 and 42.9% (3/7) with placebo, commonest being hypokalemia, pulmonary inflammation, respiratory failure, and deep venous thrombosis (n=1) with ALMB-0166 and hypokalemia (n=2), hyponatremia (n=1) with placebo. No deaths occurred.

### 1. TEAEs

In the ALMB-0166 (N=17), 16 subjects (94.1%) experienced at least 1 TEAE. TEAEs with an incidence ≥10% (by PT) included hyponatraemia in 6 subjects (35.3%); hypokalaemia, constipation, pyrexia, and anaemia in 5 subjects each (29.4%); white blood cell count increased, alanine aminotransferase increased, aspartate aminotransferase increased, red blood cells urine positive, and fibrin D-dimer increased in 3 subjects each (17.6%); hypoalbuminaemia, C-reactive protein increased, urinary occult blood positive, glucose urine present, diarrhoea, sinus bradycardia, urinary tract infection, procedural pain, and deep vein thrombosis in 2 subjects each (11.8%). Three subjects (17.6%) experienced CTCAE Grade ≥3 TEAEs, including 1 subject (5.9%) each of hypokalaemia, pneumonitis, respiratory failure, and deep vein thrombosis. The proportion of TEAEs occurring in each ALMB-0166 group (200 mg [or 3 mg/kg] group, 600 mg group, 1200 mg group, 2400 mg group, and 4800 mg) was 100%, 100%, 100%, 66.7%, and 100%, respectively.

In the placebo group (N=7), all 7 subjects (100%) experienced at least 1 TEAE. TEAEs with incidence ≥10% (by PT) included hyponatraemia, constipation, sinus bradycardia, and urinary tract infection, each with 3 subjects (42.9%); hypokalaemia and pyrexia, each with 2 subjects (28.6%); hyperkalaemia, hyperuricaemia, white blood cell count increased, alanine aminotransferase increased, aspartate aminotransferase increased, C-reactive protein increased, urinary occult blood positive, diarrhoea, functional gastrointestinal disorder, anaemia, cervical vertebral fracture, postoperative delirium, insomnia, deep vein thrombosis, metabolic encephalopathy, and rash, each with 1 subject (14.3%). Three subjects (42.9%) experienced CTCAE Grade ≥3 TEAEs, including hypokalemia in 2 subjects (28.6%) and hyponatremia in 1 subject (14.3%).

### 2. TEAEs Related to ALMB-0166

In the ALMB-0166 group (N=17), 6 subjects (35.3%) experienced at least 1 TEAE, all of which were CTCAE Grade 1, including sinus bradycardia, atrial fibrillation, alanine aminotransferase increased, red blood cells urine positive, urinary occult blood positive, glucose urine present, aspartate aminotransferase increased, reticulocyte percentage increased, reticulocyte count increased, and infusion related reaction, each occurring in 1 subject (5.9%). The incidence of TEAEs related to ALMB-0166 in each group was 50.0%, 0, 33.3%, 66.7%, and 33.3%, respectively.

In the placebo group (N=7), 2 subjects (28.6%) experienced at least 1 TEAE, both were sinus bradycardia (CTCAE Grade 1).

### 3. SAEs

Only 1 subject (5.9%) in the ALMB-0166 group experienced an SAE; the PT was deep vein thrombus, determined to be unlikely related to the study drug.

### 4. Laboratory Test and Other Safety-Related Observation Results

Compared to the placebo group, no increase in the incidence of abnormalities in hematology, blood chemistry, urinalysis, coagulation function, troponin, physical examination, ECG, or imaging examinations was observed in any of the ALMB-0166 groups.

### 5. Immunogenicity

Only in the 200 mg (or 3 mg/kg) group, the 600 mg group, and the placebo group each, 1 subject was ADA-negative at baseline and became ADA-positive after baseline. After the subject received drug therapy, the incidence of ADA positivity was low, not significantly associated with dose, and the titers of ADA-positive samples were low. Therefore, the immunogenicity risk of ALMB-0166 was low.

### V. DISCUSSION AND OVERALL CONCLUSIONS

### A. Discussion

This was a multi-center, randomized, double-blind, placebo-controlled, single-dose, dose-escalation Phase I clinical study to evaluate the safety, tolerability, and PK characteristics of ALMB-0166 in patients with acute spinal cord injury. In the study adult subjects with spinal cord injury at segment CA or below (ASIA Grade B or C) and injury within 72 hours were enrolled and assigned to various dose escalation groups of the ALMB-0166 group or placebo group according to the principles of protocol design. A total of 25 subjects were successfully screened and enrolled; 24 subjects (96.0%) received treatment, with 4 subjects each in the 200 mg (or 3 mg/kg) and 600 mg groups, 3 subjects each in the 1200 mg, 2400 mg, and 4800 mg groups, and 7 subjects in the placebo group. The demographics and baseline disease characteristics were basically balanced among the groups. The safety, efficacy, PK, and immunogenicity results of this study are discussed below.

### 1. Safety

ALMB-0166, as a monoclonal antibody against the human Cx43 hemichannel, currently has no similar drugs on the market domestically or internationally. However, ALMB-0166 has demonstrated high safety and tolerability in preclinical studies and in the first human study in healthy subjects in Australia. The preclinical results of repeated dosing in mice and cynomolgus monkeys showed that at doses ≤250 mg/kg, no test article-related systemic toxicity reactions or toxic target organs were observed, and no local irritation reactions related to the test article were found at the injection site. In the first Phase I study in Australia, 12 (42.9%) subjects who received ALMB-0166 experienced TEAEs, all of which were mild or moderate, and the vast majority were unrelated to the study drug (AlaMab Therapeutics, Inc. 2019; Protocol No.: ALMB-0166-AU-101). In this Phase I study conducted on subjects with acute spinal cord injury, ALMB-0166 continued to demonstrate good safety. The incidence of TEAEs was 94.1% (100% in the placebo group), and the vast majority (82.4%) were of CTCAE Grades 1-2. The incidence of TEAEs related to the study drug was 35.3% (28.6% in the placebo group), all of which were CTCAE Grade 1. The types of occurrences were relatively dispersed, with no AE types occurring in more than 2 subjects. No deaths, SAEs related to the study drug, or other significant AEs (TEAEs leading to dose interruption or permanent discontinuation) occurred. ALMB-0166 did not exhibit significant safety risks, and its safety and tolerability were good.

### 2. Efficacy

ALMB-0166, as a monoclonal antibody against human Cx43 hemichannels, can prevent the opening of Cx43 hemichannels induced by cell membrane injury, inhibit the release of pro-inflammatory factors from astrocytes, and minimize the spread of secondary injury. In this Phase I study on subjects with acute spinal cord injury, multiple dosage groups of ALMB-0166 showed greater mean changes from baseline on various indices of ASIA sensory scores at the D56 visit compared to the placebo group, including ASIA total sensory score (600 mg and 1200 mg groups), ASIA total sensory score—left (600 mg group), ASIA total sensory score—right (200 mg [or 3 mg/kg] group, 600 mg group, 1200 mg, and 2400 mg groups), total pinprick score (200 mg [or 3 mg/kg] group, 600 mg group, and 1200 mg group), and light touch total score (600 mg and 1200 mg groups). At the same time, this trend was also reflected in various motor score indices, where the mean changes from baseline at the D56 visit in all dose groups of the study were better than those in the placebo group, including total motor score (2400 mg group), total motor score—left (2400 mg group), total motor score—right (200 mg [or 3 mg/kg] group, 2400 mg group), upper limb total motor score (200 mg [or 3 mg/kg] group, 1200 mg group, and 2400 mg group), and lower limb total motor score (2400 mg group). For injury grading, the 1200 mg and 2400 mg groups showed a more significant trend of improvement compared to the placebo group. The above suggests that ALMB-0166 has a certain effect in improving the recovery of sensory and motor function in acute spinal cord injury. At the same time, for pain relief, the 1200 mg and 2400 mg groups also showed a better trend compared to the placebo group. The preliminary efficacy of ALMB-0166 observed in subjects with acute spinal cord injury in this study provides preliminary data support for further exploration of ALMB-0166 in the indication of acute spinal cord injury in the future.

### 3. Pharmacokinetics

In this study, after a single intravenous infusion of 3 mg or 200 mg to 4800 mg of ALMB-0166 in subjects, the in vivo exposure increased with the increase of the administered dose, exhibiting basically linear PK characteristics. The median Tₘₐₓ of ALMB-0166 in serum across dose groups was 1.03-2.01 hours, the arithmetic mean of V was 4.00-6.34 L (approximately equal to plasma volume), the arithmetic mean of t_{1/2} was 149.86-201.24 hours, and the arithmetic mean of CL was 14.29-30.94 mL/h. This is similar to the PK results in Phase I healthy humans and conforms to the PK characteristics of monoclonal antibodies.

### 4. Immunogenicity

Compared to 1 subject of ADA-negative in the placebo group, only in the 200 mg (or 3 mg/kg) group and the 600 mg group in the ALMB-0166 group, there was 1 subject each who was ADA-negative at baseline but ADA-positive after baseline. The incidence of ADA-negative was low, showed no obvious dose relationship, and the titers of ADA-positive samples were low, suggesting that the immunogenicity risk of ALMB-0166 was low. This result is consistent with the high immunogenicity safety with no ADA-positive subjects observed in the Australian Phase I study (AlaMab Therapeutics, Inc. 2019; Protocol No.: ALMB-0166-AU-101).

Based on the above safety, efficacy, and PK results, 600 mg, 1200 mg, and 2400 mg are recommended as therapeutic doses for subsequent Phase II studies of ALMB-0166.

### B. Conclusion

ALMB-0166 treatment for subjects with acute spinal cord injury showed high safety and tolerability. The incidence of TEAEs related to the study drug was low, all being CTCAE Grade 1. No TEAEs leading to death and permanent discontinuation occurred, nor were there any SAEs related to ALMB-0166.

ALMB-0166 improved the recovery of sensory motor function and pain relief in subjects with acute spinal cord injury.

Within the dose range of 200 mg (or 3 mg/kg) to 4800 mg, ALMB-0166 exhibited approximately linear PK characteristics in subjects.

ALMB-0166 posed a low risk of immunogenicity in subjects with acute spinal cord injury.

TABLE 31
Abbreviations

| Abbreviation | Definition |
|---|---|
| ADA | Antidrug Antibody |
| AE | Adverse Event |
| ASIA | American Spinal Injury Association |
| ATC | Anatomical Therapeutic Chemical (ATC) Classification |
| ATP | Adenosine Triphosphorus |
| AUC | Area under Curve |
| AUC₀₋ₜ | Area under the Curve from Zero to the Time Point |
| AUC_{0-inf} | Area under the Curve from Zero to Infinite |
| AUC₀₋ₗₐₛₜ | Area under the Plasma Concentration-time Curve from Zero |
| | to the Last Measurable Concentration |
| BQL | Below the Quantization Limit |
| C | Carotid |
| CI | Confidence Interval |
| CL | Clearance |
| CL/F | Apparent Clearance |
| Cₘₐₓ | Maximum Concentration |
| CT | Computerised Tomography |
| CTCAE | Common Adverse Event Evaluation Criteria |
| CV | Variability |
| Cx43 | Connexin 43 |
| DNA | Deoxyribonucleotide |
| EC50 | Half Maximal Effective Concentration |
| eCRF | Electronic Case Report Form |
| E/T | |
| FAS | Full Analysis Set |
| GCP | Good Clinical Practice |
| GFAP | Glial Fibrillary Acidic Protein |
| HBV | Hepatitis B Virus |
| HCV | Hepatitis C Virus |
| HIV | Human Immunodeficiency Virus |
| ICF | Informed Consent Form |
| ICH | International Conference on Harmonisation of |
| | Technical Requirements for Registration of |
| | Pharmaceuticals for Human Use |
| IFN-γ | Interferon-γ |
| IgG1 | Immunoglobulin G1 |
| IL | Interleukin |
| IS | Immunogenicity Set |
| IWRS | Interactive Web Response System |
| Max | Maximum |
| Mean | Mean |
| MedDRA | Medical Dictionary for Regulatory Activities |
| Median | Median |
| Min | Minimum |
| MRI | Magnetic Resonance Imaging |
| NCI-CTCAE | National Cancer Institute Common Terminology Criteria for |
| | Adverse Events |
| NMPA | National Medical Products Administration |
| NOAEL | No observed adverse effect level |
| NSE | Neurone-Specific Enolase |
| PD | Pharmacodynamics |
| PDS | Pharmacodynamics Set |
| PK | Pharmacokinetics |
| PKCS | Pharmacokinetic Concentration Set |
| PKPS | Pharmacokinetic Parameter Set |
| PN | Preferred Name |
| PopPK | Population Pharmacokinetics |
| PT | Preferred Term |
| QA | Quality Assurance |
| RNA | Ribonucleotides |
| RPR | Rapid plasma reagin test |
| SAE | Serious Adverse Event |
| S-100 | Central nervous system-specific protein |
| SD | Standard deviation |
| SE | Standard Error |
| SOC | System organ class |
| SOP | Standard Operating Procedure |
| SRC | Safety Review Committee |
| SS | Safety Set |
| t_{1/2} | Elimination half-life |
| TEAE | Treatment-Emergent Adverse Event |
| TGF | Transforming growth factor |
| Tₘₐₓ | Time to Maximum Plasma Concentration |
| TNF | Tumour necrosis factor |
| TRUST | Tolulized Red Unheated Serum Test |
| V | Volume of Distribution |
| WHO Drug | World Health Organization Drug Dictionary |
| λz | Terminal Rate Constant |
| % AUCₑₓ | Percentage of total AUC extrapolated |

TABLE 32
Sequence Table.

| SEQ | | |
|---|---|---|
| ID | | |
| NO | Description | Amino Acid Sequence |
| 1 | HCDR1 | GYTFTSYY |
| 2 | HCDR2 | INPSNAGT |
| 3 | HCDR3 | TREGNPYYTMNY |
| 4 | LCDR1 | QSLLNSGNQKTY |
| 5 | LCDR2 | GAS |
| 6 | LCDR3 | QNDHSYPFT |
| 7 | VH sequence | EVQLVQSGAEVKKPGASVKVSCKASGYTFTSYYMYWVRQAPGQGLEWIGGINPSNAGTNFNEKFKNRATLT |
| | | VDKSTSTAYMELSSLRSEDTAVYYCTREGNPYYTMNYWGQGTLVTVSS |
| 8 | VL sequence | DIVMTQSPDSLAVSLGERATISCKSSQSLLNSGNQKTYLAWYQQKPGQPPKLLIYGASTRESGVPDRFSGSGSG |
| | | TDFTLTISSLQAEDVAVYYCQNDHSYPFTFGQGTKLEIK |
| 9 | Heavy chain of | EVQLVQSGAEVKKPGASVKVSCKASGYTFTSYYMYWVRQAPGQGLEWIGGINPSNaGTNFNEKFKNRATLT |
| | Ab#K | VDKSTSTAYMELSSLRSEDTAVYYCTREGNPYYTMNYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTYICNVNHKPSNTKVDKKV |
| | | EPKSCDKTHTCPPCPAPEAAGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQGNVFSCSVMHEAL |
| | | HNHYTQKSLSLSPGK |
| 10 | Light chain | DIVMTQSPDSLAVSLGERATISCKSSQSLLNSGNQKTYLAWYQQKPGQPPKLLIYGASTRESGVPDRFSGSGSG |
| | | TDFTLTISSLQAEDVAVYYCQNDHSYPFTFGQGTKLEIKRTVAAPSVFIFPPSDEQLKSGTASVVCLLNNFYPR |
| | | EAKVQWKVDNALQSGNSQESVTEQDSKDSTYSLSSTLTLSKADYEKHKVYACEVTHQGLSSPVTKSFNRGEC |
| 11 | Heavy chain of | EVQLVQSGAEVKKPGASVKVSCKASGYTFTSYYMYWVRQAPGQGLEWIGGINPSNgGTNFNEKFKNRATLT |
| | Ab#E | VDKSTSTAYMELSSLRSEDTAVYYCTREGNPYYTMNYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSRWQEGNVFSCSVMHEALHNH |
| | | YTQKSLSLSLGK |
| 12 | Heavy chain of | EVQLVQSGAEVKKPGASVKVSCKASGYTFTSYYMYWVRQAPGQGLEWIGGINPSNaGTNFNEKFKNRATLT |
| | Ab#G | VDKSTSTAYMELSSLRSEDTAVYYCTREGNPYYTMNYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSRWQEGNVFSCSVMHEALHNH |
| | | YTQKSLSLSLGK |
| 13 | Heavy chain of | EVQLVQSGAEVKKPGASVKVSCKASGYTFTSYYMYWVRQAPGQGLEWIGGINPSNgGTNFNEKFKNRATLT |
| | Ab#I | VDKSTSTAYMELSSLRSEDTAVYYCTREGNPYYTMNYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTYICNVNHKPSNTKVDKKV |
| | | EPKSCDKTHTCPPCPAPEAAGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQGNVFSCSVMHEAL |
| | | HNHYTQKSLSLSPGK |
| 14 | Heavy chain of | EVQLVQSGAEVKKPGASVKVSCKASGYTFTSYYMYWVRQAPGQGLEWIGGINPSNgGTNFNEKFKNRATLT |
| | Ab#C | VDKSTSTAYMELSSLRSEDTAVYYCTREGNPYYTMNYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQGNVFSCSVMHEAL |
| | | HNHYTQKSLSLSPGK |
| 15 | Heavy chain of | EVQLVQSGAEVKKPGASVKVSCKASGYTFTSYYMYWVRQAPGQGLEWIGGINPSNgGTNFNEKFKNRATLT |
| | Ab#M | VDKSTSTAYMELSSLRSEDTAVYYCTREGNPYYTMNYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKVDKRV |
| | | ESKYGPPCPPCPAPEFEGGPSVFLFPPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSRWQEGNVFSCSVMHEALHNH |
| | | YTQKSLSLSLGK |
| 16 | Heavy chain of | EVQLVQSGAEVKKPGASVKVSCKASGYTFTSYYMYWVRQAPGQGLEWIGGINPSNaGTNFNEKFKNRATLT |
| | Ab#O | VDKSTSTAYMELSSLRSEDTAVYYCTREGNPYYTMNYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKVDKRV |
| | | ESKYGPPCPPCPAPEFEGGPSVFLFPPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSRWQEGNVFSCSVMHEALHNH |
| | | YTQKSLSLSLGK |
| 17 | Heavy chain of | EVQLVQSGAEVKKPGASVKVSCKASGYTFTSYYMYWVRQAPGQGLEWIGGINPSNgGTNFNEKFKNRATLT |
| | Ab#Q | VDKSTSTAYMELSSLRSEDTAVYYCTREGNPYYTMNYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKVDKRV |
| | | ESKYGPPCPPCPAPEAAGGPSVFLFPPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLTVDKSRWQEGNVFSCSVMHEALHN |
| | | HYTQKSLSLSLGK |
| 18 | Heavy chain of | EVQLVQSGAEVKKPGASVKVSCKASGYTFTSYYMYWVRQAPGQGLEWIGGINPSNaGTNFNEKFKNRATLT |
| | Ab#S | VDKSTSTAYMELSSLRSEDTAVYYCTREGNPYYTMNYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKVDKRV |
| | | ESKYGPPCPPCPAPEAAGGPSVFLFPPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLTVDKSRWQEGNVFSCSVMHEALHN |
| | | HYTQKSLSLSLGK |
| 19 | Epitope | FLSRPTEKTI |

1. A method for treating spinal cord injury (SCI) in a subject in need thereof, comprising administering to the subject an anti-connexin 43 (Cx43) antibody, wherein the anti-Cx43 antibody comprises the following heavy chain CDR sequences and light chain CDR sequences:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5; and
LCDR3: SEQ ID NO: 6.

2. (canceled)

3. The method of claim 1, wherein the anti-Cx43 antibody is administered at a dosage between 200 mg to 4800 mg.

4. The method of claim 3, wherein the dosage is about 200 mg, about 600 mg, about 1200 mg, about 2400 mg, or about 4800 mg.

5. The method of claim 4, wherein the dosage is about 200 mg.

6. The method of claim 4, wherein the dosage is about 600 mg.

7. The method of claim 4, wherein the dosage is about 1200 mg.

8. The method of claim 4, wherein the dosage is about 2400 mg.

9. The method of claim 4, wherein the dosage is about 4800 mg.

10. The method of claim 1, wherein the subject is a human.

11. The method of claim 1, wherein the SCI is
(i) C3-C7 SCI;
(ii) acute SCI;
(iii) complete SCI; or
(iv) incomplete SCI.

12. (canceled)

13. (canceled)

14. (canceled)

15. The method of claim 1, wherein the anti-Cx43 antibody is administered intravenously or subcutaneously.

16. The method of claim 15, wherein the anti-Cx43 antibody is administered intravenously.

17. The method of claim 16, where in the anti-Cx43 antibody is administered within 60 minutes.

18. The method of claim 1, wherein at least one evaluation indicator of spinal cord injury is improved after the anti-Cx43 antibody is administered.

19. The method of claim 18, wherein the at least one indicator is selected from a group consisting of: ASIA motor score, total motor score (right), total motor score (left), upper limb motor score, upper limb motor score, ASIA sensatory score, injury grading, pinprick score, light touch score, and pain visual analogue scale.

20. The method of claim 1, wherein the subject
(i) does not experience severe adverse event after the anti-Cx43 antibody is administered, or
(ii) is anti-drug antibody (ADA) negative after the anti-Cx43 antibody is administered.

21. (canceled)

22. The method of claim 1, wherein the anti-Cx43 antibody comprises a heavy chain variable sequence of SEQ ID NO: 7, and/or a light chain variable sequence of SEQ ID NO: 8.

23. The method of claim 1, wherein the anti-Cx43 antibody comprises a heavy chain sequence of any one of SEQ ID NOs: 9 and 11-18, and/or light chain sequence of SEQ ID NO: 10.

24. The method of claim 1, wherein the anti-Cx43 antibody comprises heavy chain sequence of SEQ ID NO: 9, and/or light chain sequence of SEQ ID NO: 10.

25. The method of claim 1, wherein the anti-Cx43 antibody blocks the opening of Cx43 hemichannel in the spinal cord astrocytes of the subject.
